(19) United States
(12) Patent Application Publication
(10) Pub. No.: US 2010/0063041 A1
(43) Pub. Date: Mar. 11, 2010

(54) NOVEL PHENYLPROPIONIC ACID DERIVATIVES AS PEROXISOME PROLIFERATOR-ACTIVATED GAMMA RECEPTOR MODULATORS, METHOD OF THE SAME, AND PHARMACEUTICAL COMPOSITION COMPRISING THE SAME

(76) Inventors: Ho-Sang Moon, Gyeonggi-do (KR); Moo-Hi Yoo, Seoul (KR); Soon-Hoe Kim, Gyeonggi-do (KR); Joong-In Lim, Gyeongi-do (KR); Moon-Ho Son, Gyeonggi-do (KR); Mi-Kyung Kim, Gyeonggi-do (KR); Chang-Yell Shin, Seoul (KR); Jin-Kwan Kim, Gyeonggi-do (KR); Sang-Kuk Park, Gyeonggi-do (KR); Yu-Na Chae, Gyeonggi-do (KR); Hyun-Joo Shim, Gyeonggi-do (KR); Sun-Ho Jeon, Seoul (KR); Hae-Sun Kim, Gyeonggi-do (KR); Gil-Tae Wie, Gyeonggi-do (KR); Dong-Hwan Kim, Gyeonggi-do (KR); Byung-Kyu Lee, Gyeonggi-do (KR); Chan-Sun Park, Gyeonggi-do (KR); Byung-Nak Ahn, Seoul (KR); Eunkyung Kim, Seoul (KR); Myung-Ho Bae, Gyeonggi-do (KR); Young-Ah Shin, Gyeonggi-do (KR); Youn Hur, Gyeonggi-do (KR); Chun-Ho Lee, Seoul (KR); Hyun-Ho Choi, Gyeonggi-do (KR); Bongtae Kim, Gyeonggi-do (KR); Wonee Chong, Gyeonggi-do (KR)

Correspondence Address: THE NATH LAW GROUP, 112 South West Street, Alexandria, VA, 22314, US

(21) Appl. No.: 12/449,979

(22) PCT Filed: Mar. 7, 2008
(86) PCT No.: PCT/KR2008/001322

§ 371 Date: Sep. 4, 2009

(30) Foreign Application Priority Data
Mar. 7, 2007 (KR) 10-2007-0022681
Mar. 7, 2008 (KR) 10-2008-0021695

(51) Int. Cl. A61K 31/538; C07D 333/16; A61K 31/381; C07D 413/02; A61K 31/5377; A61K 31/5355; C07D 277/20; A61K 31/426; C07D 409/02; A61K 31/501; C07D 498/02; A61P 3/10; A61P 3/06

(52) U.S. Cl. 514/230.5; 549/78; 514/438; 544/146; 514/231.5; 544/96; 514/228.8; 548/202; 514/365; 544/238; 514/252.01; 544/105

(57) ABSTRACT

The present invention provides a novel phenylpropionic acid derivative and a PPAR-γ modulator comprising the same as an active ingredient. The phenylpropionic acid derivative of the present invention has modulatory action on function of PPAR-γ and then exhibits hypoglycemic, hypolipidemic and insulin resistance-reducing effects on PPAR-mediated diseases or disorders. Therefore, the present invention is prophylactically or therapeutically effective for diabetes and metabolic diseases.

### TECHNICAL FIELD

The present invention relates to novel compounds represented by formula 1, and preparation and use thereof:

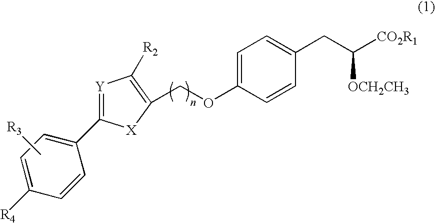

The compound of formula 1 has modulatory effects on peroxisome proliferator-activated receptor gamma (hereinafter, referred to as “PPAR-γ”) and therefore can be effective for hypoglycemic (blood glucose-lowering) effects, hypolipidemic (blood lipid-lowering) effects, and alleviation of insulin resistance.

### BACKGROUND ART

Diabetes mellitus is a chronic metabolic disease which has a prevalence rate of nearly 5% among populations of industrialized countries. An incidence rate of Type 2 diabetes mellitus (formerly called non-insulin-dependent diabetes mellitus, NIDDM), which accounts for 90% or higher of diabetic conditions, is gradually increasing with generalization of high-calorie diet and advanced country-type lifestyle habits (Rondinone et al, Exp Opin Ther Targets (2005) 9:415-419). Type 2 diabetic patients frequently suffer from attendant diseases such as hyperglycemia, hyperlipidemia, atherosclerosis and obesity. Particularly, a primary etiological factor of Type 2 diabetes mellitus is insulin resistance. That is, the incidence of Type 2 diabetes mellitus is initiated with manifestation of insulin resistance at the early stage, followed by hypoinsulinaemia due to dysfunction of pancreatic beta cells.

PPAR-γ is a transcriptional activator or transactivator that mediates adipogenic differentiation. Rosiglitazone and pioglitazone drugs, which are synthetic ligands for PPAR-γ, have been clinically proven to be excellent therapeutic agents that are capable of regulating an elevated blood glucose level by enhancing insulin sensitivity of Type 2 diabetic patients to thereby alleviate insulin resistance. However, conventional glitazone drugs entail adverse side effects such as potential risks of edema and weight gain in practical clinical applications and development of cardiac hypertrophy in preclinical animal models, even though these drugs exhibit excellent drug efficacy. Consequently, these problems of glitazone drugs are major obstacles to the choice of a first-line drug for the treatment of Type 2 diabetes mellitus (Acton et al., Bioorg Med Chem Lett (2005) 15:357-362). To this end, there has been a strong need for development of the next-generation PPAR-γ agonist which is pharmacologically safe and ideal in the nature of a drug.

As recently reported in the literature (Reifel-Miller et al., Mol Endocrinol (2005) 19:1593-1605), a selective PPAR-γ modulator is a drug that elicits a relatively low PPAR-γ transcriptional activity, as compared to a ligand species which theoretically exhibits 100% transcriptional activity, such as rosiglitazone, and that has hypoglycemic effects simultaneously with reduction of the above-mentioned adverse side effects. Further, improvement of insulin sensitivity does not necessarily require 100% activation of PPAR-γ.

Further, the selective PPAR-γ modulator nTZDpa shows different adipocyte-specific gene expression patterns than those of a ligand that exhibits 100% transcriptional activity, such as rosiglitazone. In addition, when nTZDpa in combination with high-fat diet was administered to animals for 13 weeks in animal experiments using C57BL/6J mice, comparable drug efficacy was achieved with significantly low weight gain of adipose tissues while not causing significant differences in blood glucose and insulin levels, as compared to a control group fed with high-fat diet and a group treated with a ligand exhibiting 100% transcriptional activity. Further, body weight gain and cardiac weight gain were not reported which may be usually observed in the ligand-treated group exhibiting 100% transcriptional activity [Changes in cardiac weight: LF, +0.140.01 g/HF+TZDfa, +0.230.02 g (P<0.05)/HF+nTZDpa, +0.150.01 g (P>0.05)] (Berger et al., Mol Endocrinol (2003) 17:662-676).

Selective PPAR-γ modulators reported hitherto are known to show differences in binding capacity with cofactors or drug responsiveness different from that of conventional PPAR-γligands through tissue-specific gene expression regulation or the like. Metaglidasen that is currently under phase II/III clinical trials was reported to have weak or substantially no binding activity with cofactors such as N-CoR, SMRT, p300, CBP, and Trap220, as compared to rosiglitazone (Allen et al., Diabetes (2006) 55:2523-2533). Further, it was reported that INT-131 exhibits attenuated binding capacity for Trap220 (Abstract 659-P, 64^{th} ADA, 2004). Trap220 was reported to serve as an essential factor in the PPAR-γ-mediated adipogenic differentiation process (G E et al., Nature (2002) 417:563-567). Therefore, these properties of Trap220 are understood as a mechanism factor responsible for reduced adverse side effects on body weight, as compared to conventional drugs.

Therapeutic effects of metaglidasen and INT-131 were demonstrated in animal models. Specifically, administration of these drugs resulted in amelioration in development of edema and weight gain (Abstract 44-OR, 65^{th} ADA, 2005; and Abstract 659-P, 64^{th} ADA, 2004). 12-week clinical results of metaglidasen showed that co-administration of metaglidasen with insulin exhibits excellent drug efficacy with a 0.7% decrease of glycosylated hemoglobin as compared to a non-treated control group, in conjunction with a 21% reduction in the blood triglyceride level. However, there was no significant difference in the body weight (+0.6 kg vs. +1.3 kg in control group; P>0.05) and an edema incidence rate (7.2% vs. 20% in control group; P>0.05) between the drug-treated group and the control group. From these results, it can be seen that relief of adverse side effects was proven in preclinical animal models as well as in clinical trials (Abstract 44-OR, 65^{th} ADA, 2005).

When rosiglitazone and pioglitazone were administered to patients, the onset of edema was observed in 10 to 15% of patients within 3 months from after the first application of drugs (Mudaliar et al., Endocr Pract, 2003 (9):406-16; and Page et al., Pharmacotherapy, 2003 (23):945-54). Further, since chronic administration of one year or more is inevitable due to intrinsic characteristics of concerned diseases, administration of the drug is accompanied by weight gain, simultaneously with poor compliance with drug regimens in clinical applications, consequently posing the possibility of further progress into risk factors of other diseases.

Accordingly, if a selective PPAR-γ modulator having reduced adverse side effects while retaining the desired therapeutic efficacy of the drug is quickly commercialized in the market, conventional PPAR-γ agonist markets will undergo rapid changes by replacement with such a PPAR-γ drug. Further, if such a selective PPAR-γ modulator drug is applicable as a first-line drug of choice for early-stage diabetes, due to decreased adverse side effects of the drug, the existing PPAR-γ agonist market will expand even further.

As a result of a variety of extensive and intensive studies and experiments to solve the problems as described above, the inventors of the present invention succeeded in synthesis of a novel compound which is capable of achieving improved insulin sensitivity and efficient control of the blood glucose level through the selective modulation of PPAR-γ while simultaneously reducing adverse side effects that were shown by conventional drugs. The present invention has been completed based on this finding.

### DISCLOSURE OF THE INVENTION

### Technical Problem

It is an object of the present invention to provide a novel phenylpropionic acid derivative having modulatory activity on PPAR-γ and a method for preparing the same.

It is another object of the present invention to provide a PPAR-γ modulator comprising a phenylpropionic acid derivative as an active ingredient, which has reduced adverse side effects of conventional PPAR drugs and which is therapeutically effective for PPAR-mediated diseases and has hypoglycemic, hypolipidemic and insulin resistance-reducing activity.

### Technical Solution

In accordance with an aspect of the present invention, the above and other objects can be accomplished by the provision of a novel phenylpropionic acid derivative represented by formula 1 and a pharmaceutically acceptable salt thereof.

In accordance with another aspect of the present invention, there is provided a use of a PPAR-γ modulator comprising a phenylpropionic acid derivative represented by formula 1 or a pharmaceutically acceptable salt thereof, as an active ingredient.

In accordance with a further aspect of the present invention, there is provided a novel compound having a structure of formula 1 and a pharmaceutically acceptable salt thereof. Further, racemates, optical isomers and pharmaceutically acceptable salts of a compound of formula 1 fall within the scope of the present invention.

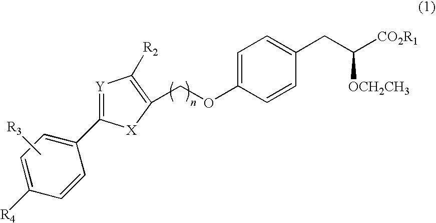

wherein:

R₁ is hydrogen, ethyl, or an alkali metal;

R₂ is hydrogen or methyl;

X is S or O;

Y is N or CH;

R₃ is hydrogen, lower alkyl or lower alkoxy;

R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and

n is an integer of 1 to 5.

In formula 1,

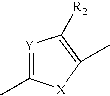

is preferably selected from:

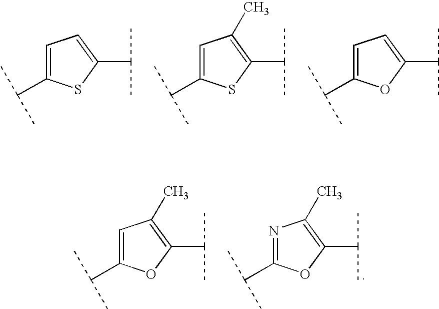

Preferably, lower alkyl is selected from methyl, ethyl and isopropyl; lower alkoxy is selected from methoxy and ethoxy; and halide is selected from Cl, F and Br.

Preferably, alkylcarbamoyl is selected from:

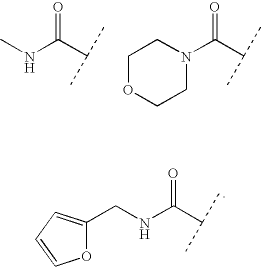

Preferably, oxadiazole is selected from:

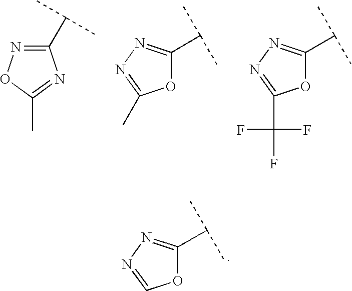

Preferably, isoxazole is selected from:

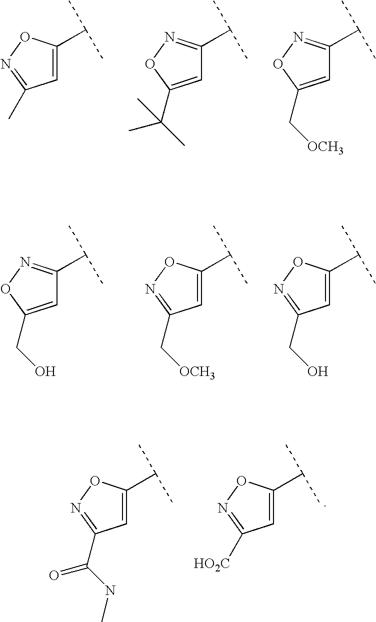

Preferably, tetrazole is selected from:

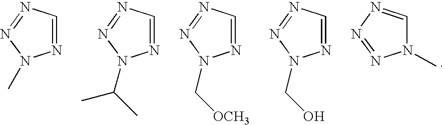

Representative examples of compounds in accordance with the present invention may include the following compounds:
(S)-2-ethoxy-3-(4-((5-(3-methoxyphenyl)-3-methylthiophen-2-yl)methoxy)phenyl)propionic acid,
(S)-2-ethoxy-3-(4-((5-(4-fluorophenyl)-3-methylthiophen-2-yl)methoxy)phenyl)propionic acid,
(S)-3-(4-((5-(3,4-dimethoxyphenyl)-3-methylthiophen-2-yl)methoxy)phenyl)-2-ethoxypropionic acid,
(S)-2-ethoxy-3-(4-((5-(4-methoxyphenyl)-3-methylthiophen-2-yl)methoxy)phenyl)propionic acid,
(S)-2-ethoxy-3-(4-((5-(4-ethylphenyl)-3-methylthiophen-2-yl)methoxy)phenyl)propionic acid,
(S)-2-ethoxy-3-(4-((3-methyl-5-(4-(trifluoromethyl)phenyl)thiophen-2-yl)methoxy)phenyl)propionic acid,
(S)-2-ethoxy-3-(4-((3-methyl-5-p-phenylthiophen-2-yl)methoxy)phenyl)propionic acid,
(S)-2-ethoxy-3-(4-((3-methyl-5-(4-(trifluoromethoxy)phenyl)thiophen-2-yl)methoxy)phenyl)propionic acid,
(S)-2-ethoxy-3-(4-((5-(4-isopropylphenyl)-3-methylthiophen-2-yl)methoxy)phenyl)propionic acid,
(S)-2-ethoxy-3-(4-((3-methyl-5-phenylthiophen-2-yl)methoxy)phenyl)propionic acid,
(S)-3-(4-((5-(4-cyanophenyl)-3-methylthiophen-2-yl)methoxy)phenyl)-2-ethoxypropionic acid,
(S)-3-(4-((5-(4-acetylphenyl)-3-phenylthiophen-2-yl)methoxy)phenyl)-2-ethoxypropionic acid,
(S)-3-(4-((5-(4-acetamidophenyl)-3-methylthiophen-2-yl)methoxy)phenyl)-2-ethoxypropionic acid,
(S)-2-ethoxy-3-(4-((3-methyl-5-(4-(N-methylacetamido)phenyl)thiophen-2-yl)methoxy)phenyl)propionic acid,
(S)-3-(4-((5-(4-benzoylphenyl)-3-methylthiophen-2-yl)methoxy)phenyl)-2-ethoxypropionic acid,
(S)-2-ethoxy-3-(4-((5-(4-(furan-2-yl-methylcarbamoyl)phenyl)-3-methylthiophen-2-yl)methoxy)phenyl)propionic acid,
(S)-2-ethoxy-3-(4-((3-methyl-5-(4-(morpholine-4-carbonyl)phenyl)thiophen-2-yl)methoxy)phenyl)propionic acid,
(S)-2-ethoxy-3-(4-((3-methyl-5-(4-(morpholinosulfonyl)phenyl)thiophen-2-yl)methoxy)phenyl)propionic acid,
(S)-3-(4-((5-(4-(5,6-dihydro-4H-1,3-oxazin-2-yl)phenyl)-3-methylthiophen-2-yl)methoxy)phenyl)-2-ethoxypropionic acid,
(S)-2-ethoxy-3-(4-((3-methyl-5-(4-morpholinophenyl)thiophen-2-yl)methoxy)phenyl)propionic acid,
(S)-2-ethoxy-3-(4-((3-methyl-5-(4-(2-methylthiazol-4-yl)phenyl)thiophen-2-yl)methoxy)phenyl)propionic acid,
(S)-2-ethoxy-3-(4-((3-methyl-5-(4-(1-methyl-6-oxo-1,4,5,6-tetrahydropyridazin-3-yl)phenyl)thiophen-2-yl)methoxy)phenyl)propionic acid,
(S)-3-(4-((5-(4-(2H-benzo[b][1,4]oxazin-3-yl)phenyl)-3-methylthiophen-2-yl)methoxy)phenyl)-2-ethoxypropionic acid,
(S)-3-(4-((5-(4-(1,2,3-thiadiazol-4-yl)phenyl)-3-methylthiophen-2-yl)methoxy)phenyl)-2-ethoxypropionic acid,
(S)-2-ethoxy-3-(4-((3-methyl-5-(4-(5-methyl-1,2,4-oxadiazol-3-yl)phenyl)thiophen-2-yl)methoxy)phenyl)propionic acid,
(S)-2-ethoxy-3-(4-((3-methyl-5-(4-(5-methyl-1,3,4-oxadiazol-2-yl)phenyl)thiophen-2-yl)methoxy)phenyl)propionic acid,
(S)-2-ethoxy-3-(4-((3-methyl-5-(4-(5-(trifluoromethyl)-1,3,4-oxadiazol-2-yl)phenyl)thiophen-2-yl)methoxy)phenyl)propionic acid,
(S)-3-(4-((5-(4-(1,3,4-oxadiazol-2-yl)phenyl)-3-methylthiophen-2-yl)methoxy)phenyl)-2-ethoxypropionic acid,
(S)-2-ethoxy-3-(4-((3-methyl-5-(4-(2-methyl-2H-tetrazol-5-yl)phenyl)thiophen-2-yl)methoxy)phenyl)propionic acid,
(S)-2-ethoxy-3-(4-((3-methyl-5-(4-(1-methyl-1H-tetrazol-5-yl)phenyl)thiophen-2-yl)methoxy)phenyl)propionic acid,
(S)-2-ethoxy-3-(4-((5-(4-(2-isopropyl-2H-tetrazol-5-yl)phenyl)-3-methylthiophen-2-yl)methoxy)phenyl)propionic acid,
(S)-2-ethoxy-3-(4-((5-(4-(2-(methoxymethyl)-2H-tetrazol-5-yl)phenyl)-3-methylthiophen-2-yl)methoxy)phenyl)propionic acid,
(S)-2-ethoxy-3-(4-((5-(4-(2-(hydroxymethyl)-2H-tetrazol-5-yl)phenyl)-3-methylthiophen-2-yl)methoxy)phenyl)propionic acid,
(S)-3-(4-((5-(4-(4,5-dimethyloxazol-2-yl)phenyl)-3-methylthiophen-2-yl)methoxy)phenyl)-2-ethoxypropionic acid,
(S)-2-ethoxy-3-(4-((5-(4-(5-(hydroxymethyl)isoxazol-3-yl)phenyl)-3-methylthiophen-2-yl)methoxy)phenyl)propionic acid,
(S)-2-ethoxy-3-(4-((5-(4-(5-(methoxymethyl)isoxazol-3-yl)phenyl)-3-methylthiophen-2-yl)methoxy)phenyl)propionic acid,
(S)-5-(4-(5-((4-(2-carbonyl-2-ethoxyethyl)phenoxy)methyl)-4-methylthiophen-2-yl)phenyl)isoxazole-3-carboxylic acid,
(S)-2-ethoxy-3-(4-((3-methyl-5-(4-(3-(methylcarbamoyl)isoxazol-5-yl)phenyl)thiophen-2-yl)methoxy)phenyl)propionic acid,
(S)-2-ethoxy-3-(4-((5-(4-(3-(hydroxymethyl)isoxazol-5-yl)phenyl)-3-methylthiophen-2-yl)methoxy)phenyl)propionic acid,
(S)-2-ethoxy-3-(4-((5-(4-(3-(methoxymethyl)isoxazol-5-yl)phenyl)-3-methylthiophen-2-yl)methoxy)phenyl)propionic acid,
(S)-2-ethoxy-3-(4-((3-methyl-5-(4-(3-methylisoxazol-5-yl)phenyl)thiophen-2-yl)methoxy)phenyl)propionic acid,
lithium (S)-2-ethoxy-3-(4-((3-methyl-5-(4-(3-methylisoxazol-5-yl)phenyl)thiophen-2-yl)methoxy)phenyl)propanoate,
sodium (S)-2-ethoxy-3-(4-((3-methyl-5-(4-(3-methylisoxazol-5-yl)phenyl)thiophen-2-yl)methoxy)phenyl)propanoate,
potassium (S)-2-ethoxy-3-(4-((3-methyl-5-(4-(3-methylisoxazol-5-yl)phenyl)thiophen-2-yl)methoxy)phenyl)propanoate,
(S)-2-ethoxy-3-(4-((5-(4-(3-methylisoxazol-5-yl)phenyl)furan-2-yl)methoxy)phenyl)propionic acid,
(S)-2-ethoxy-3-(4-((3-methyl-5-(4-(3-methylisoxazol-5-yl)phenyl)furan-2-yl)methoxy)phenyl)propionic acid,
(S)-2-ethoxy-3-(4-((4-methyl-2-(4-(3-methylisoxazol-5-yl)phenyl)thiazol-5-yl)methoxy)phenyl)propionic acid,
(S)-3-(4-((5-(4-(5-tert-butylisoxazol-3-yl)phenyl)-3-methylthiophen-2-yl)methoxy)phenyl)-2-ethoxypropionic acid,
lithium (S)-3-(4-((5-(4-(5-tert-butylisoxazol-3-yl)phenyl)-3-methylthiophen-2-yl)methoxy)phenyl)-2-ethoxypropanoate,
sodium (S)-3-(4-((5-(4-(5-tert-butylisoxazol-3-yl)phenyl)-3-methylthiophen-2-yl)methoxy)phenyl)-2-ethoxypropanoate,
potassium (S)-3-(4-((5-(4-(5-tert-butylisoxazol-3-yl)phenyl)-3-methylthiophen-2-yl)methoxy)phenyl)-2-ethoxypropanoate,
(S)-3-(4-((5-(4-(5-tert-butylisoxazol-3-yl)phenyl)furan-2-yl)methoxy)phenyl)-2-ethoxypropionic acid,
(S)-3-(4-((5-(4-(5-tert-butylisoxazol-3-yl)phenyl)-3-methylfuran-2-yl)methoxy)phenyl)-2-ethoxypropionic acid, and
(S)-3-(4-((2-(4-(5-tert-butylisoxazol-3-yl)phenyl)-4-methylthiazol-5-yl)methoxy)phenyl)-2-ethoxypropionic acid.

The phenylpropionic acid derivatives in accordance with the present invention have an asymmetric carbon center, and may be present in the form of racemates and corresponding optical isomers. All kinds of these isomers fall within the scope of the present invention. The optical isomers were given optical selectivity via enzymatic reactions of racemic intermediates. The enzyme used in synthesis of the compounds in accordance with the present invention was Viscozyme-L (Novozyme) as disclosed in Korean Patent Application No. 2006-66440.

Racemic resolution for producing optically active isomers of a compound represented by formula 1 may be carried out by a conventional resolution method known in the art. For example, a base of the compound of formula 1 is reacted with an optically active acid to form a salt of the compound of formula 1, and then dextro (right) and levo (left) forms of optical isomers are then separated by fractional crystallization. Examples of acids suitable for resolution of the compound of formula 1 may include optically active forms of tartaric acid, ditolyltartaric acid, dibenzoyltartaric acid, malic acid, mandelic acid and camphorsulfonic acid and any optically active acid known in the related art. In this case, more biologically and optically active stereoisomeric forms of the compound of formula 1 are preferably separated.

The compound of formula 1 in accordance with the present invention include pharmaceutically acceptable salts thereof, for example salts with inorganic acids such as hydrochloric acid, hydrobromic acid, phosphoric acid, and sulfuric acid; salts with organic carboxylic acids such as acetic acid, trifluoroacetic acid, citric acid, maleic acid, oxalic acid, succinic acid, benzoic acid, tartaric acid, fumaric acid, mandelic acid, ascorbic acid, and malic acid; salts with sulfonic acids such as methanesulfonic acid, and p-toluenesulfonic acid; salts with alkali metals such as sodium, potassium, and lithium; salts with organic amines such as ethanolamine; and salts with any acid known in the art.

Further, the present invention provides a method for preparing a compound represented by formula 1 or a pharmaceutically acceptable salt thereof.

The preparation method of the present invention comprises (1) reacting a compound of formula 2 with a compound of formula 3, 4, 5 or 6 to form a compound of formula 7, 8, 9 or 10; and (2) reacting the compound of formula 7, 8, 9 or 10 with a boron compound of formula 11 to form a compound of formula 1 wherein R₁ is ethyl. When R₁ is hydrogen, the method may further comprise hydrolysis of the ethyl ester compound of Step 2 by the reaction with a base. When R₁ is an alkali metal, the method may further comprise reacting the hydrolysate, obtained from reaction of the ester compound of Step 2 with the base, with an alkali metal salt to prepare a desired compound of formula 1:

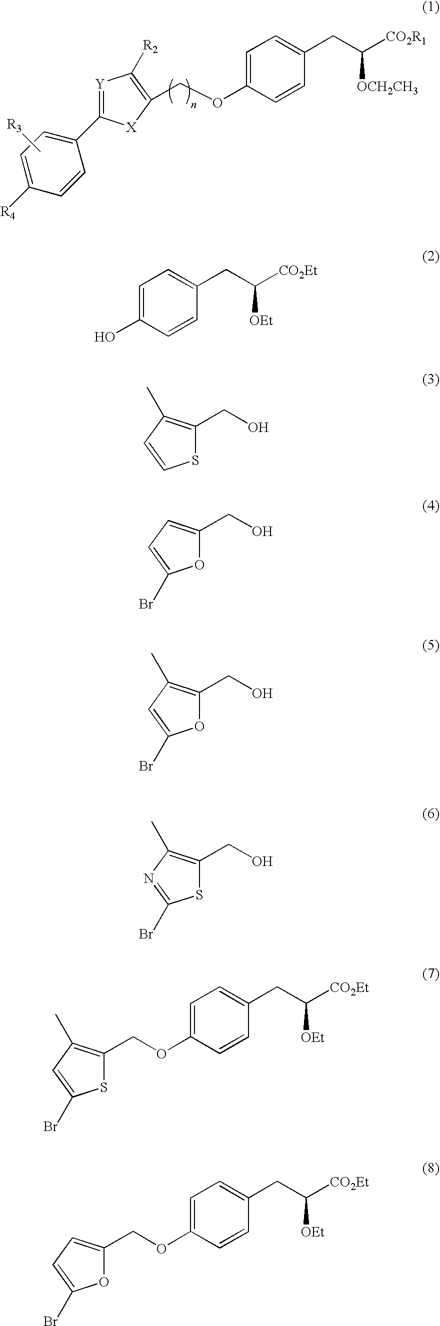

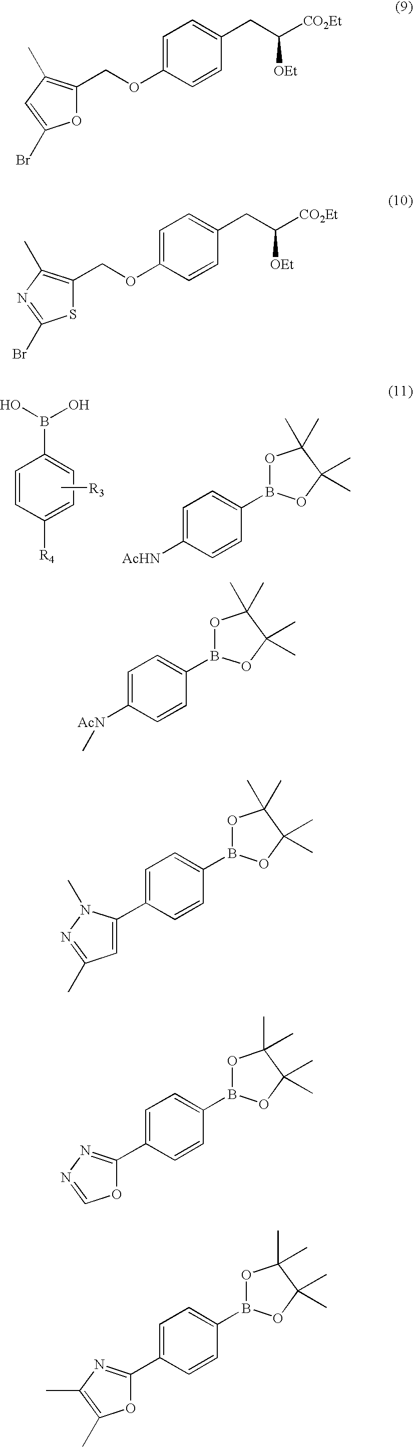

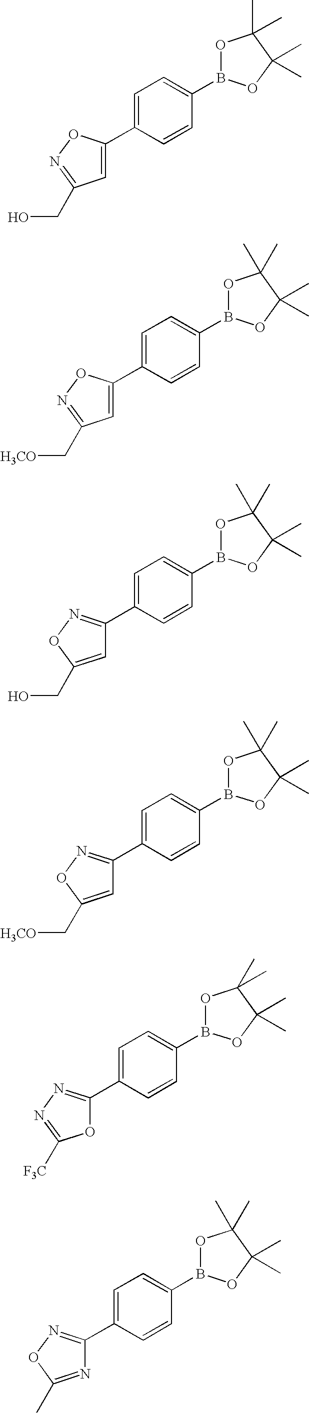

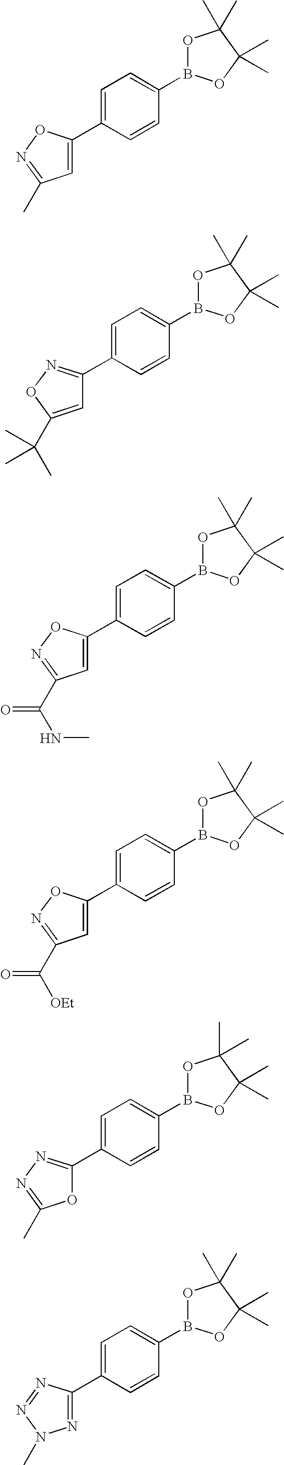

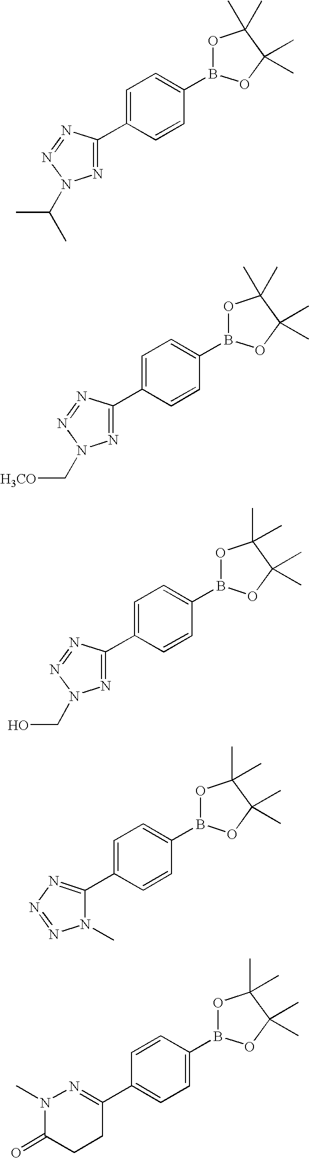

In formula 1, R₁ is hydrogen, ethyl, or an alkali metal; R₂ is hydrogen or methyl; X is S or O; Y is N or CH; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁ alkyl, C₁ alkoxy, hydroxy, amino, trifluoromethyl, phenyl, benzyl, benzoyl, furan, thiophene, piperidine and morpholine; and n is an integer of 1 to 5.

Specifically, the compound of formula 1 in accordance with the present invention may be prepared by Step 1: nucleophilic substitution of Compound 2 with Compound 3 through the Mitsunobu reaction to form an ether bond, followed by bromination of the reaction product with N-bromosuccinimide to form Compound 7; and Step 2: Suzuki coupling of Compound 7 using boronic acid or dioxaborolan of formula 11 and a palladium catalyst to form a carbon-carbon bond. The resulting compound of formula 1 may be a compound of formula 1-1.

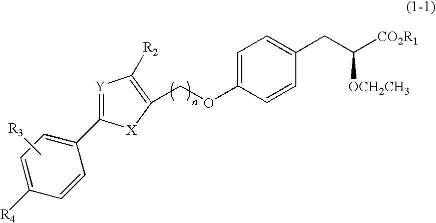

wherein R₁ is ethyl; X is S; Y is CH; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 4 through the Mitsunobu reaction to form Compound 8 via formation of an ether bond, and Step 2: Suzuki coupling of Compound 8 using boronic acid or dioxaborolan of formula 11 and a palladium catalyst to form a carbon-carbon bond. The resulting compound of formula 1 may be a compound of formula 1-2.

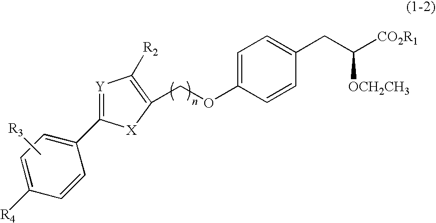

wherein R₁ is ethyl; X is O; Y is CH; R₂ is hydrogen; R₃ and R₄ are as defined in formula 1, and n is 1.

Alternatively, the compound of formula 1 may be prepared by Step 1: nucleophilic substitution of Compound 2 with Compound 5 through the Mitsunobu reaction to form Compound 9 via formation of an ether bond, and Step 2: Suzuki coupling of Compound 9 using boronic acid or dioxaborolan of formula 11 and a palladium catalyst to form a carbon-carbon bond. The resulting compound of formula 1 may be a compound of formula 1-3.

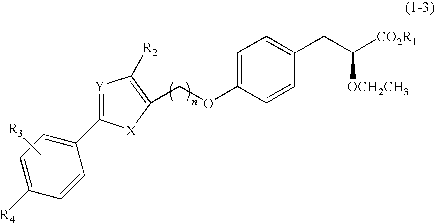

wherein R₁ is ethyl; X is O; Y is CH; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 6 through the Mitsunobu reaction to form Compound 10 via formation of an ether bond, and Step 2: Suzuki coupling of Compound 10 using boronic acid or dioxaborolan of formula 11 and a palladium catalyst to form a carbon-carbon bond. The resulting compound of formula 1 may be a compound of formula 1-4.

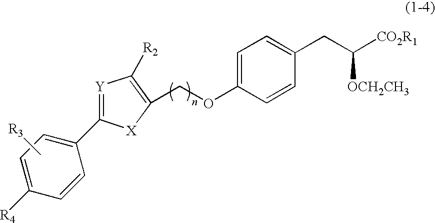

wherein R₁ is ethyl; X is S; Y is N; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

The method may further comprise hydrolysis of the reaction product of Step 2 after reaction with a boron compound to thereby form a compound of formula 1 wherein R₁ is hydrogen. From the compound of formula 1 wherein R₁ is hydrogen, a compound of formula 1 wherein R₁ is ethyl may be obtained as an intermediate.

Alternatively, the compound of formula 1 may be prepared by Step 1: nucleophilic substitution of Compound 2 with Compound 3 through the Mitsunobu reaction to form an ether bond, followed by bromination of the reaction product with N-bromosuccinimide to form Compound 7, and Step 2: Suzuki coupling of Compound 7 using boronic acid or dioxaborolan of formula 11 and a palladium catalyst to form a carbon-carbon bond, followed by hydrolysis. The resulting compound of formula 1 may be a compound of formula 1-5. When a carbon-carbon bond of Step 2 is formed in the preparation process of Compound 1-5, a compound of formula 1 wherein R₁ is ethyl may be obtained as an intermediate.

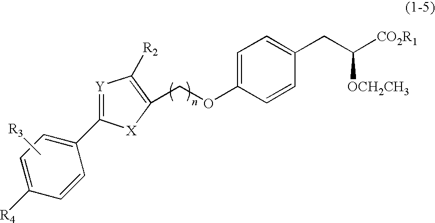

wherein R₁ is hydrogen; X is S; Y is CH; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 4 through the Mitsunobu reaction to form Compound 8 via formation of an ether bond, and Step 2: Suzuki coupling of Compound 8 using boronic acid or dioxaborolan of formula 11 and a palladium catalyst to form a carbon-carbon bond, followed by hydrolysis. The resulting compound of formula 1 may be a compound of formula 1-6. When a carbon-carbon bond of Step 2 is formed in the preparation process of Compound 1-6, a compound of formula 1 wherein R₁ is ethyl may be obtained as an intermediate.

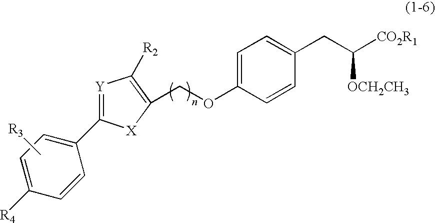

wherein R₁ is hydrogen; X is O; Y is CH; R₂ is hydrogen; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 5 through the Mitsunobu reaction to form Compound 9 via formation of an ether bond, and Step 2 Suzuki coupling of Compound 9 using boronic acid or dioxaborolan of formula 11 and a palladium catalyst to form a carbon-carbon bond, followed by hydrolysis. The resulting compound of formula 1 may be a compound of formula 1-7. When a carbon-carbon bond of Step 2 is formed in the preparation process of Compound 1-7, a compound of formula 1 wherein R₁ is ethyl may be obtained as an intermediate.

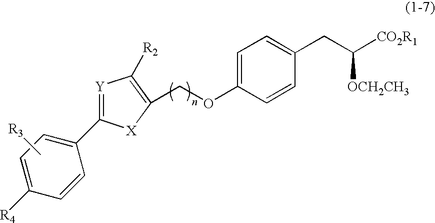

wherein R₁ is hydrogen; X is O; Y is CH; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 6 through the Mitsunobu reaction to form Compound 10 via formation of an ether bond, and Step 2: Suzuki coupling of Compound 10 using boronic acid or dioxaborolan of formula 11 and a palladium catalyst to form a carbon-carbon bond, followed by hydrolysis. The resulting compound of formula 1 may be a compound of formula 1-8. When a carbon-carbon bond of Step 2 is formed in the preparation process of Compound 1-8, a compound of formula 1 wherein R₁ is ethyl may be obtained as an intermediate.

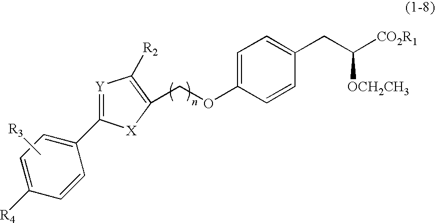

wherein R₁ is hydrogen; X is S; Y is N; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

The method may further comprise reacting the hydrolysate of Step 2, obtained from hydrolysis of the reaction product after reaction with a boron compound, with sodium, lithium or potassium ethyl-2 hexanoate to prepare a compound of formula 1 wherein R₁ is an alkali metal. During preparation of the compound of formula 1 wherein R₁ is an alkali metal, a compound of formula 1 wherein R₁ is ethyl may be obtained as an intermediate.

Alternatively, the compound of formula 1 may be prepared by Step 1: nucleophilic substitution of Compound 2 with Compound 3 through the Mitsunobu reaction to form an ether bond, followed by bromination of the reaction product with N-bromosuccinimide to form Compound 7, and Step 2: Suzuki coupling of Compound 7 using boronic acid or dioxaborolan of formula 11 and a palladium catalyst to form a carbon-carbon bond, followed by hydrolysis. The resulting compound of formula 1 may be a compound of formula 1-9. When a carbon-carbon bond of Step 2 is formed in the preparation process of Compound 1-9, a compound of formula 1 wherein R₁ is ethyl may be obtained as an intermediate.

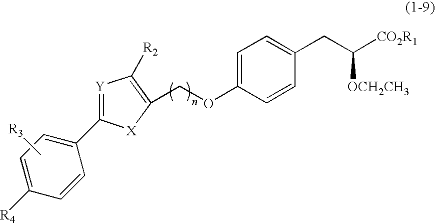

wherein R₁ is an alkali metal; X is S; Y is CH; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 4 through the Mitsunobu reaction to form Compound 8 via formation of an ether bond, and Step 2: Suzuki coupling of Compound 8 using boronic acid or dioxaborolan of formula 11 and a palladium catalyst to form a carbon-carbon bond, followed by hydrolysis. The resulting compound of formula 1 may be a compound of formula 1-10. When a carbon-carbon bond of Step 2 is formed in the preparation process of Compound 1-10, a compound of formula 1 wherein R₁ is ethyl may be obtained as an intermediate.

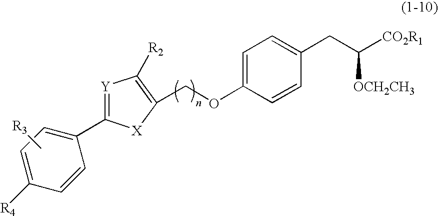

wherein R₁ is an alkali metal; X is O; Y is CH; R₂ is hydrogen; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 5 through the Mitsunobu reaction to form Compound 9 via formation of an ether bond, and Step 2: Suzuki coupling of Compound 9 using boronic acid or dioxaborolan of formula 11 and a palladium catalyst to form a carbon-carbon bond, followed by hydrolysis. The resulting compound of formula 1 may be a compound of formula 1-11. When a carbon-carbon bond of Step 2 is formed in the preparation process of Compound 1-11, a compound of formula 1 wherein R₁ is ethyl may be obtained as an intermediate.

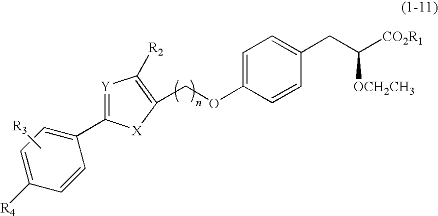

wherein R₁ is an alkali metal; X is O; Y is CH; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and n is 1.

Alternatively, the compound of formula 1 may be prepared by Step 1: nucleophilic substitution of Compound 2 with Compound 6 through the Mitsunobu reaction to form Compound 10 via formation of an ether bond, and Step 2: Suzuki coupling of Compound 10 using boronic acid or dioxaborolan of formula 11 and a palladium catalyst to form a carbon-carbon bond, followed by hydrolysis. The resulting compound of formula 1 may be a compound of formula 1-12. When a carbon-carbon bond of Step 2 is formed in the preparation process of Compound 1-12, a compound of formula 1 wherein R₁ is ethyl may be obtained as an intermediate.

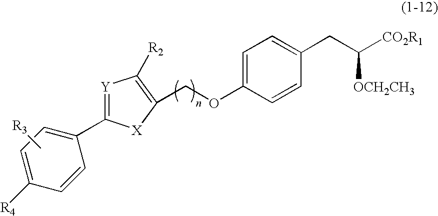

wherein R₁ is an alkali metal; X is S; Y is N; R₂ is methyl; R₃ is hydrogen, lower alkyl or lower alkoxy; R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₄ alkyl, C₁₋₆ alkoxy, hydroxy, amino, trifluoromethyl, phenyl, benzyl, benzoyl, furan, thiophene, piperidine and morpholine; and n is 1.

In individual reactions of the present invention, starting materials and reactants are added, mixed and stirred in a reaction solvent. There is no particular limit to the reaction solvent, so long as it facilitates nucleophilic substitution and hydrolysis while not having adverse effects on the reaction of interest. Examples of the reaction solvent that can be used in the present invention may include alcohols such as methanol, ethers such as dioxane and tetrahydrofuran (THF), aromatic solvents such as benzene and toluene, chlorinated hydrocarbons such as methylene chloride and dichloroethane, and organic solvents such as acetonitrile and N,N-dimethylformamide (DMF). These materials may be used alone or in any combination thereof. The reaction may be carried out at a temperature of 0 to 150° C.

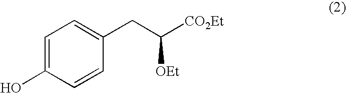

The compound of formula 2 encompasses an optical isomeric form thereof. Preferred is an (S)-form of Compound 2. Compound 2 may be prepared by condensation of commercially available 4-benzyloxybenzaldehyde as a starting material with triethyl 2-phosphonobutyrate, followed by hydrogenation. The condensation step may be carried out as a Wittig-type reaction (cf. Comprehensive Organic Synthesis vol. 1 p. 755-781, Pergamon Press) or as described in the Preparation Examples hereinafter. For example, an olefin intermediate is synthesized by reaction of reaction materials in the presence of hydrogenation products of alkali metals such as sodium hydride (NaH) and potassium hydride (KH), organolithium such as methyl lithium (CH₃Li) and butyl lithium (BuLi), alkoxides such as sodium methoxide (NaOMe), sodium ethoxide (NaOEt) and potassium t-butoxide (t-BuOK), or bases such as lithium hydroxide (LiOH) and sodium hydroxide (NaOH), which is followed by reduction of the intermediate using hydrogen gas and a Pd/C, Rh/C or Pt/C catalyst or a mixture thereof. Examples of the reaction solvent may include dioxane, acetic acid, ethyl acetate, and ethanol. Properties of the solvent are not particularly important. The reaction may be carried out under pressure of 80 psi. The catalyst is preferably 5 to 10% Pd/C, and may be used in a range of 1 to 100% w/w.

Synthesis of Compound 2 is illustrated in the following Reaction Scheme. The resulting hydrogenation product is obtained in the form of a racemic mixture. Therefore, for synthesis of an optical isomeric form, hydrolysis of Compound 2 is carried out via the selective enzymatic reaction to prepare a preferred (s)-form of carboxylic acid, followed by esterification (Mats T. Liderberg et al., Organic Process Research & Development 2004, 8, 838-845). The enzyme used in synthesis of the desired compound was Viscozyme-L (Novozyme) as disclosed in Korean Patent Application No. 2006-66440.

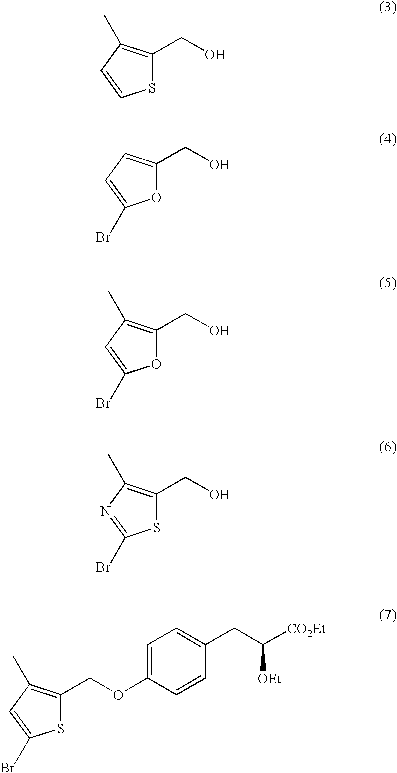

Compound 7 may be prepared by nucleophilic substitution of Compound 2 with Compound 3 through the Mitsunobu reaction (Mats T. Liderberg et al., Organic Process Research & Development 2004, 8, 838-845.) to form an ether bond, followed by bromination of the reaction product with N-bromosuccinimide (hereinafter, referred to as “NBS”) to form a desired compound.

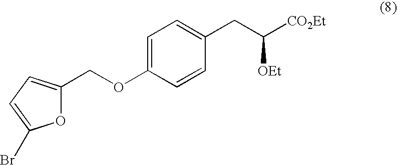

Compound 8 was prepared from Compounds 2 and 4 through the Mitsunobu reaction.

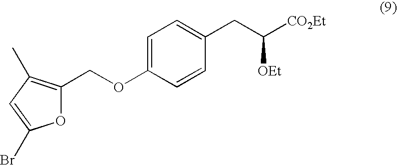

Compound 9 was prepared from Compounds 2 and 5 through the Mitsunobu reaction.

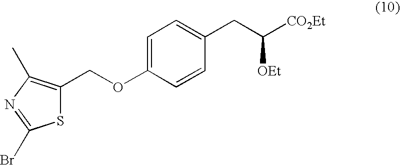

Compound 10 was prepared from Compounds 2 and 6 through the Mitsunobu reaction.

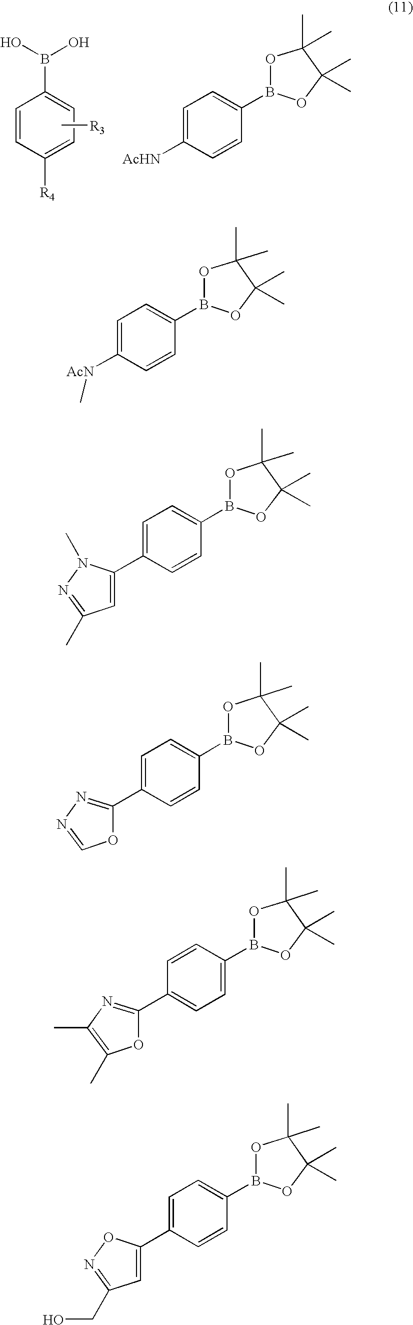

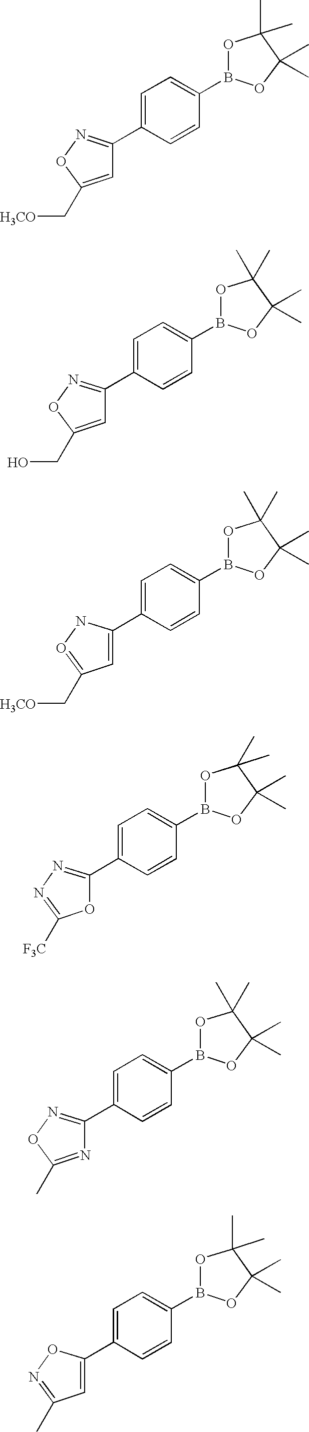

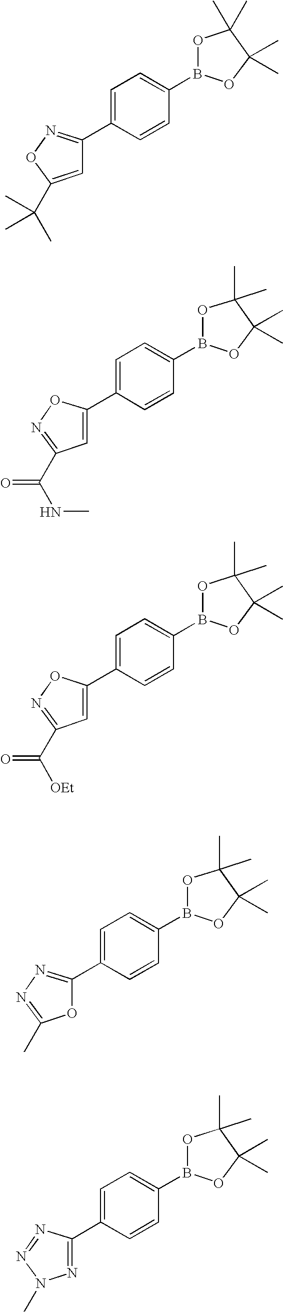

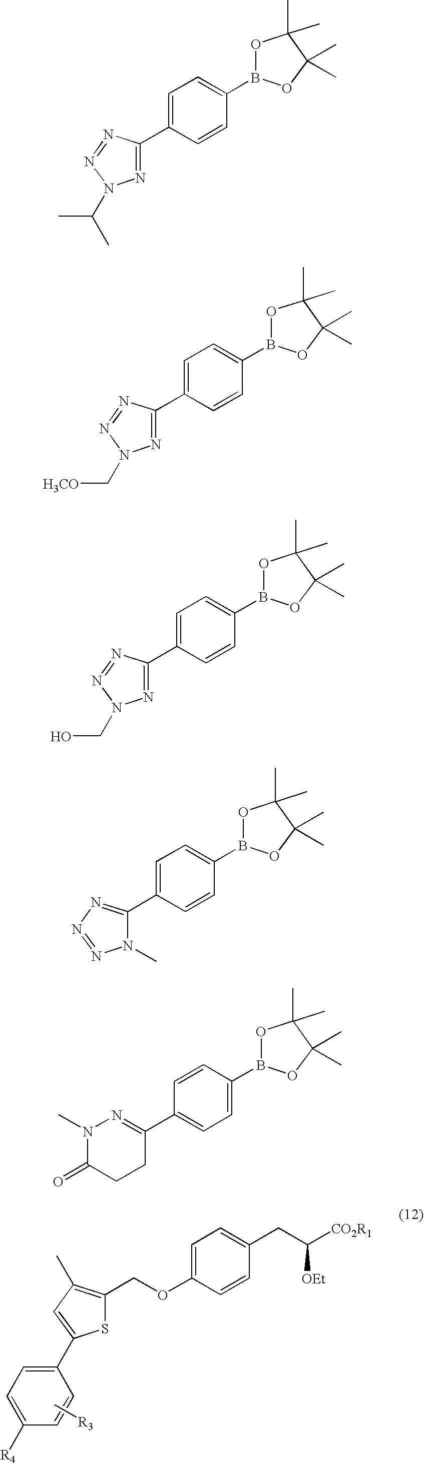

Compound 12 was prepared by formation of a carbon-carbon bond using Compounds 7 and 11 in the presence of a palladium catalyst, followed by hydrolysis of the reaction product. Further, the alkali metal salt may be prepared by reacting the hydrolyzed compound with a certain reagent.

In the aforesaid formulae, R₁, R₃, and R₄ are as defined in formula 1.

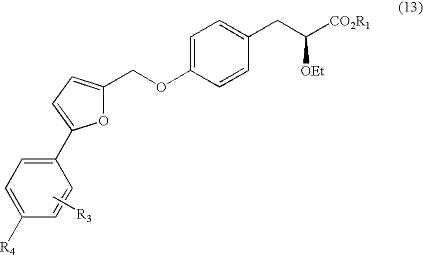

Analogously to preparation of Compound 12, Compound 13 was prepared from Compound 8.

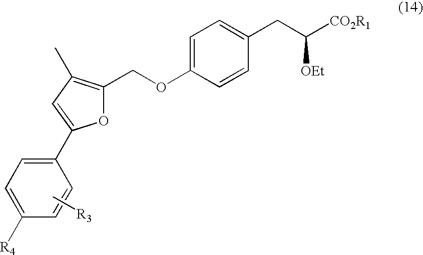

Analogously to preparation of Compound 13, Compound 14 was prepared from Compound 9.

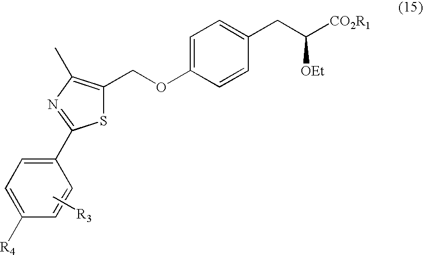

Analogously to preparation of Compound 13, Compound 15 was prepared from Compound 10.

Hereinafter, Reaction Schemes provide experimental methods of Examples and Preparation Examples which will follow.

Reaction Scheme 1 below illustrates a general method for preparation of a compound represented by formula 1.

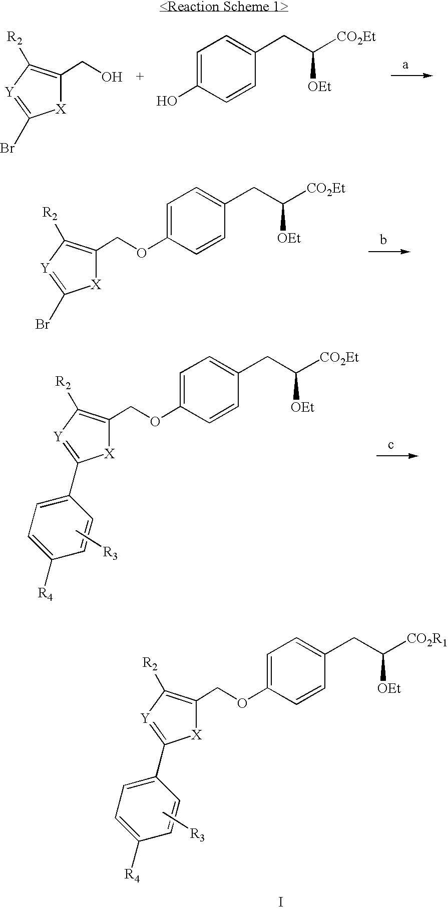

Reagents and Reaction Conditions:

a) Diisopropyl azodicarboxylate, triphenylphosphine, room temperature, 2 hours.

b) Boronic acid or 4,4,5,5-tetramethyl-1,3,2-dioxaborolan derivative, tetrakis(triphenylphosphine)palladium, cesium carbonate, dioxane, 90° C., 2 hours. Alternatively, tetrakis(triphenylphosphine)palladium, aq. potassium carbonate (K₂CO₃), toluene, ethanol, 90° C., 1 hour.

c) 1N—NaOH, ethanol, tetrahydrofuran (THF), 50° C., 1 hour.

In the present invention, the compound of formula 1 was prepared by formation of an ether bond by nucleophilic substitution through the Mitsunobu reaction, formation of a carbon-carbon bond through Suzuki coupling reaction (Suzuki A. et al., Synth. Commun. (1981), 11, 513) using boronic acid or dioxaborolan as defined in formula 11 and a palladium catalyst, and then hydrolysis of the reaction product under basic conditions to synthesize a desired form of propionic acid.

In Reaction Scheme 1, R₁, R₂, R₃, R₄, X, and Y are as defined in formula 1.

Reaction Scheme 2 below illustrates a method for preparation of Compound 2.

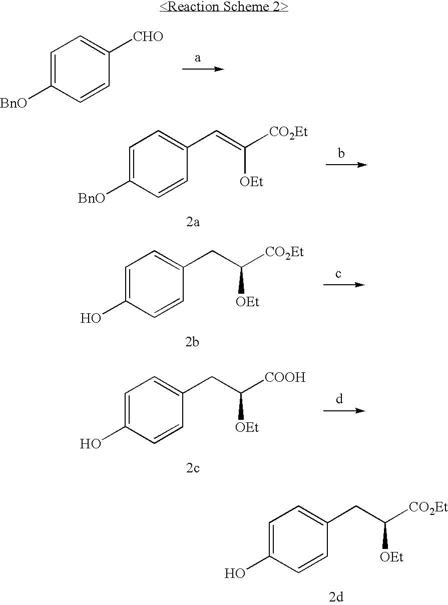

Reagents and Reaction Conditions:

a) Triethyl 2-ethoxyphosphonoacetate, potassium, t-butoxide (t-BuOH), toluene, room temperature, 3 hours.

b) H₂/10% Pd—C, ethanol (EtOH), 12 hours.

c) Viscozyme L (Viscozyme L), phosphate buffer, room temperature, 48 hours.

d) Thionyl chloride (SOCl₂), ethanol (EtOH), reflux, 3 hours.

The optical activity of Compound 2 was assayed by determining the optical purity (ee: enantiomeric excess) of the compound in the form of carboxylic acid using the following column. The optical purity of the compound was 99.54%. Analysis conditions are as follows:

Column: Shiseido Capcell Pak C18 MG 3.0×250 mm, 5 μm

Mobile phase: MeOH/H₂O=8/2, 0.1%-TEA, 0.05%-H₃PO₄.

Flow rate: 0.5 mL/min

Reaction Scheme 3 below illustrates synthesis of Compound 3.

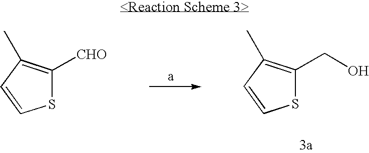

Reagents and Reaction Conditions:

a) Sodium borohydride, ethanol, room temperature, 1 hour.

Reaction Scheme 4 below illustrates synthesis of Compound 4. Compound 4 was prepared in the same manner as in Reaction Scheme 3.

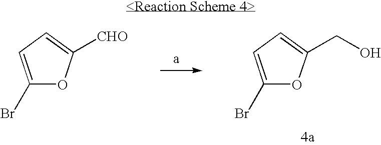

Reagents and Reaction Conditions:

a) Sodium borohydride, ethanol, room temperature, 1 hour.

Reaction Scheme 5 below illustrates synthesis of Compound 5.

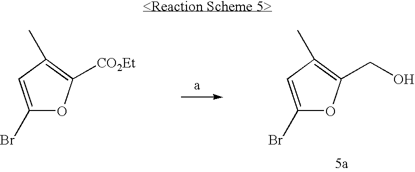

Reagents and Reaction Conditions:

a) Lithium aluminum hydride (LAH), tetrahydrofuran (THF), room temperature, 1 hour.

Reaction Scheme 6 below illustrates synthesis of Compound 6.

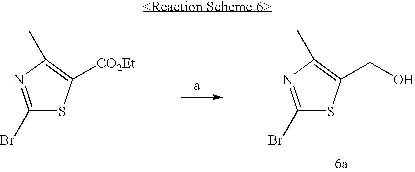

Reagents and Reaction Conditions:

a) Lithium aluminum hydride (LAH), tetrahydrofuran (THF), room temperature, 1 hour.

Reaction Scheme 7 below illustrates synthesis of Compound 7.

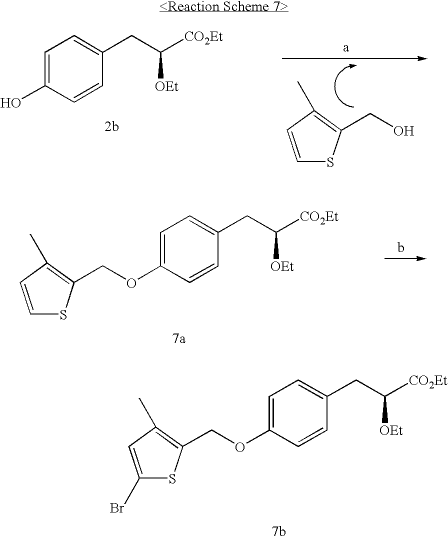

Reagents and Reaction Conditions:

a) Diisopropyl azodicarboxylate, triphenylphosphine, room temperature, 2 hours.

b) N-bromosuccinimide (NBS), N,N-dimethylformamide (hereinafter, referred to as “DMF”), room temperature, 3 hours.

Reaction Scheme 8 below illustrates synthesis of Compound 8.

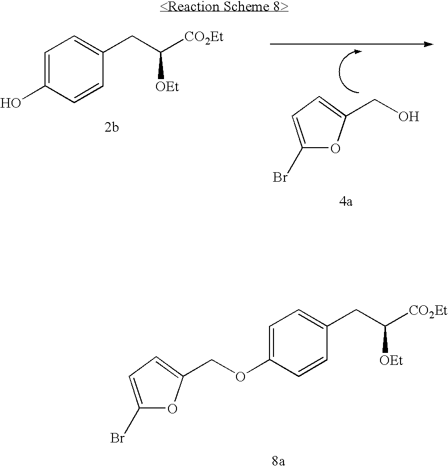

Reagents and Reaction Conditions:

Diisopropyl azodicarboxylate, triphenylphosphine, room temperature, 2 hours. Reaction Scheme 9 below illustrates synthesis of Compound 9.

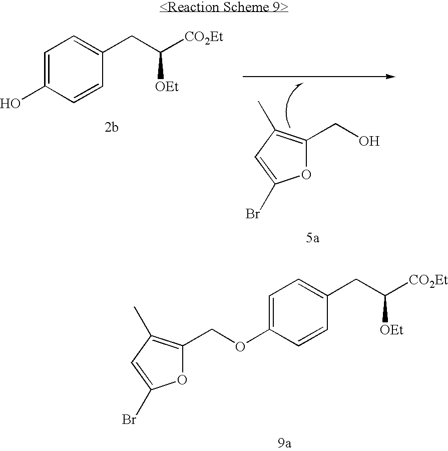

Reagents and Reaction Conditions:

Diisopropyl azodicarboxylate, triphenylphosphine, room temperature, 2 hours.

Reaction Scheme 10 below illustrates synthesis of Compound 10.

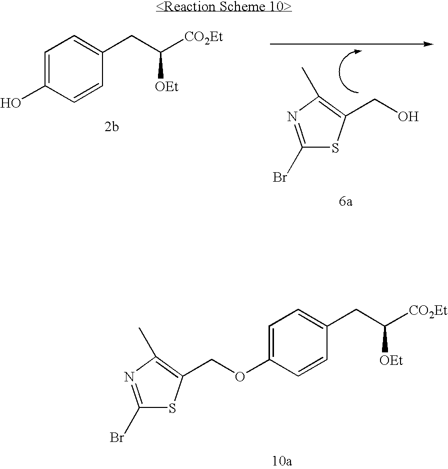

Reagents and Reaction Conditions:

Diisopropyl azodicarboxylate, triphenylphosphine, room temperature, 2 hours.

Reaction Scheme 11 below illustrates synthesis of 5-substituted-3-(4-(4,4,5,5-tetramethyl-1,3,2-dioxaborolan-2-yl)phenyl)isoxazole among compounds of formula 11.

The isoxazole compound was prepared by reacting 4-bromobenzaldehyde (as a starting material) with hydroxylamine to form an oxime compound, introducing chloride into the oxime compound via use of N-chlorosuccinimide (hereinafter, referred to as “NCS”), and reacting the resulting compound with a certain butyne compound to obtain isoxazole. Thereafter, a desired compound was prepared by replacement of bromine into dioxaborolan using bis(pinacolato)diboron.

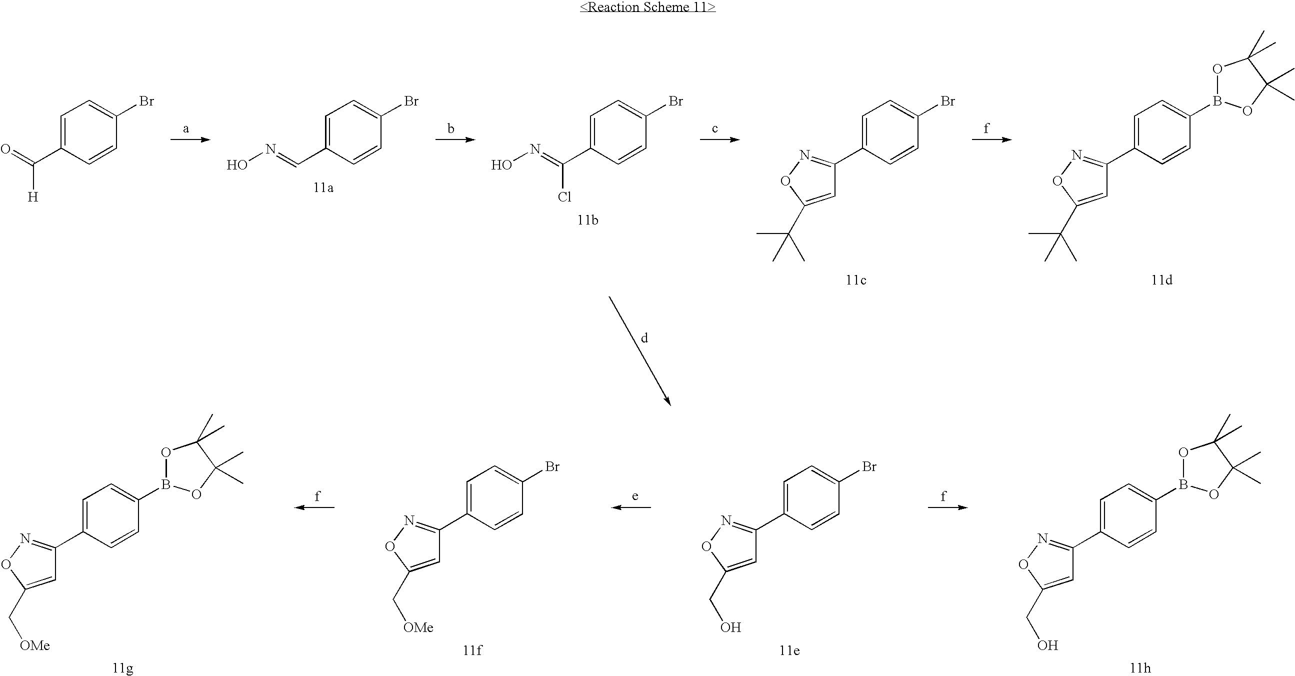

Reagents and Reaction Conditions:

a) Hydroxylamine hydrogen chloride (NH₂OH.HCl), pyridine, room temperature, 2 hours.

b) N-chlorosuccinimide (NCS), N,N-dimethylformamide (DMF), room temperature, 1 hour.

c) 3,3-dimethyl-1-butyne, triethylamine (hereinafter, referred to as “Et₃N”), methylene chloride (CH₂Cl₂), room temperature, 5 hours.

d) 2-propyn-1-ol, triethylamine (Et₃N), methylene chloride (CH₂Cl₂), room temperature, 5 hours.

e) Sodium hydride (NaH), methyl iodide (MeI), N,N-dimethylformamide (DMF), room temperature, 1 hour.

f) Bis(pinacolato)diboron, [1,1′-bis(diphenylphosphino)ferrocene]dichloropalladium (II) complex with dichloromethane (1:1), potassium acetate, dioxane, 90° C., 2 hours.

Reaction Scheme 12 below illustrates synthesis of 3-substituted-5-(4-(4,4,5,5-tetramethyl-1,3,2-dioxaborolan-2-yl)phenyl)isoxazole among compounds of formula 11.

The isoxazole compound was prepared by reacting 4-bromoacetophenone (as a starting material) with N,N-dimethylacetamide dimethyl acetal or diethyl oxalate to synthesize Compounds 12a and 12d, and reacting Compounds 12a or 12d with hydroxylamine to synthesize isoxazole compounds. Thereafter, a compound for formation of a carbon-carbon bond was synthesized by replacement of bromine of isoxazole compounds (12b, 12e, 12f, and 12i) into dioxaborolan via use of bis(pinacolato)diboron.

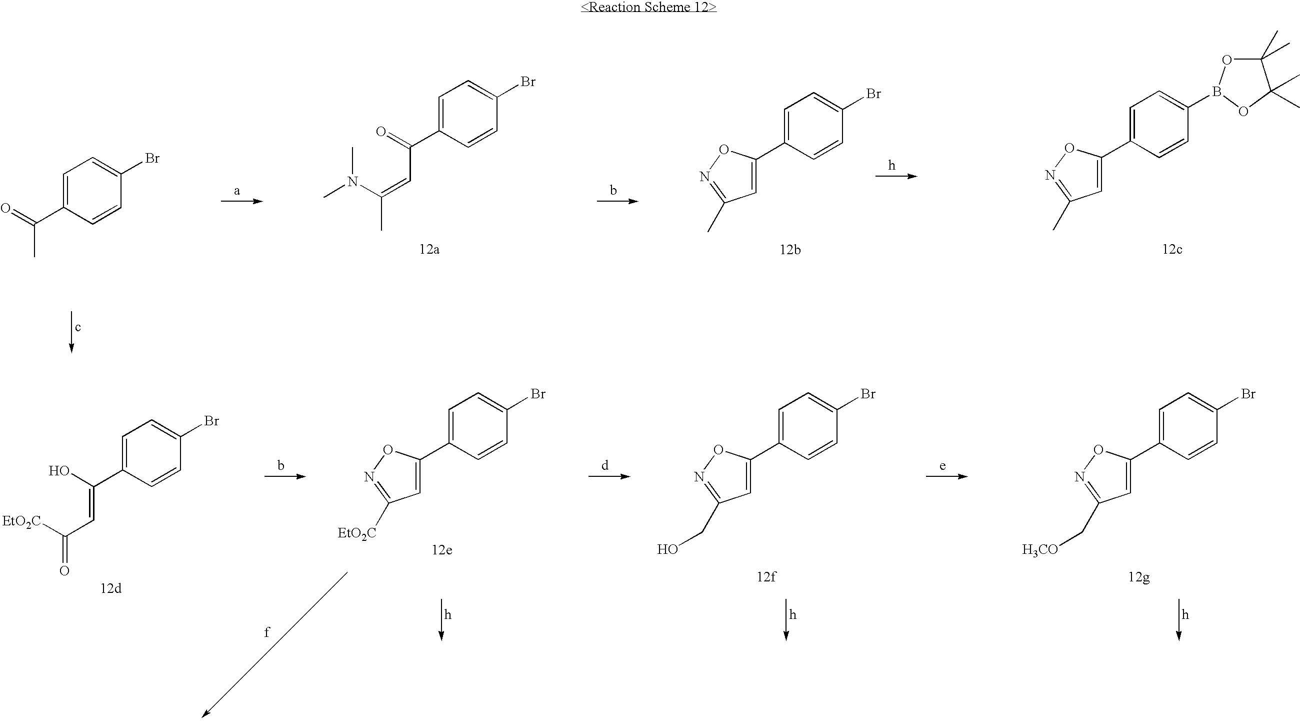

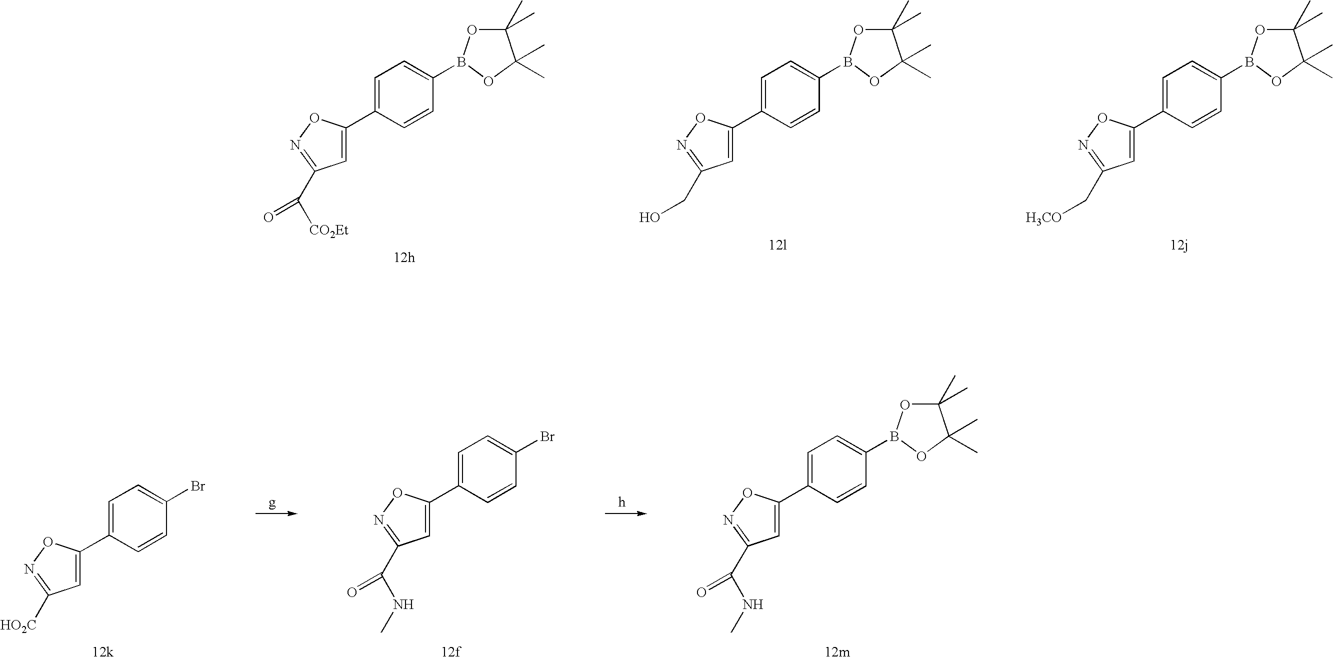

Reagents and Reaction Conditions:

a) N,N-dimethylacetamide dimethyl acetal, 1,4-dioxane, reflux, 12 hours.

b) Hydroxylamine, ethanol, reflux, 2 hours.

c) Diethyl oxalate (CO₂Et)₂), 60% sodium hydride, toluene, reflux, 1 hour.

d) Lithium aluminum hydride, tetrahydrofuran (THF), 0° C., 2 hours.

e) Sodium hydride, MeI (methyl iodide), N,N-dimethylformamide (DMF), room temperature, 1 hour.

f) 1N—NaOH, ethanol, tetrahydrofuran (THF), 60° C., 1 hour.

g) (COCl)₂ (diethyl oxalate), tetrahydrofuran (THF), reflux followed by addition of methylamine hydrochloride, triethylamine (Et₃N), tetrahydrofuran (THF), room temperature.

h) Bis(pinacolato)diboron, [1,1′-bis(diphenylphosphino)ferrocene]dichloropalladium (I) complex with dichloromethane (1:1), potassium acetate, dioxane, 90° C., 2 hours.

Reaction Scheme 13 below illustrates synthesis of a tetrazole compound among compounds of formula 11.

For this purpose, 4-bromophenyl cyanide as a starting material was reacted with sodium azide and ammonium chloride to form tetrazole. Using a certain reagent, nucleophilic substitution was made to synthesize a 1- or 2-substituted tetrazole compound. Thereafter, a compound for formation of a carbon-carbon bond was prepared by replacement of bromine of the tetrazole compound into dioxaborolan via use of bis(pinacolato)diboron.

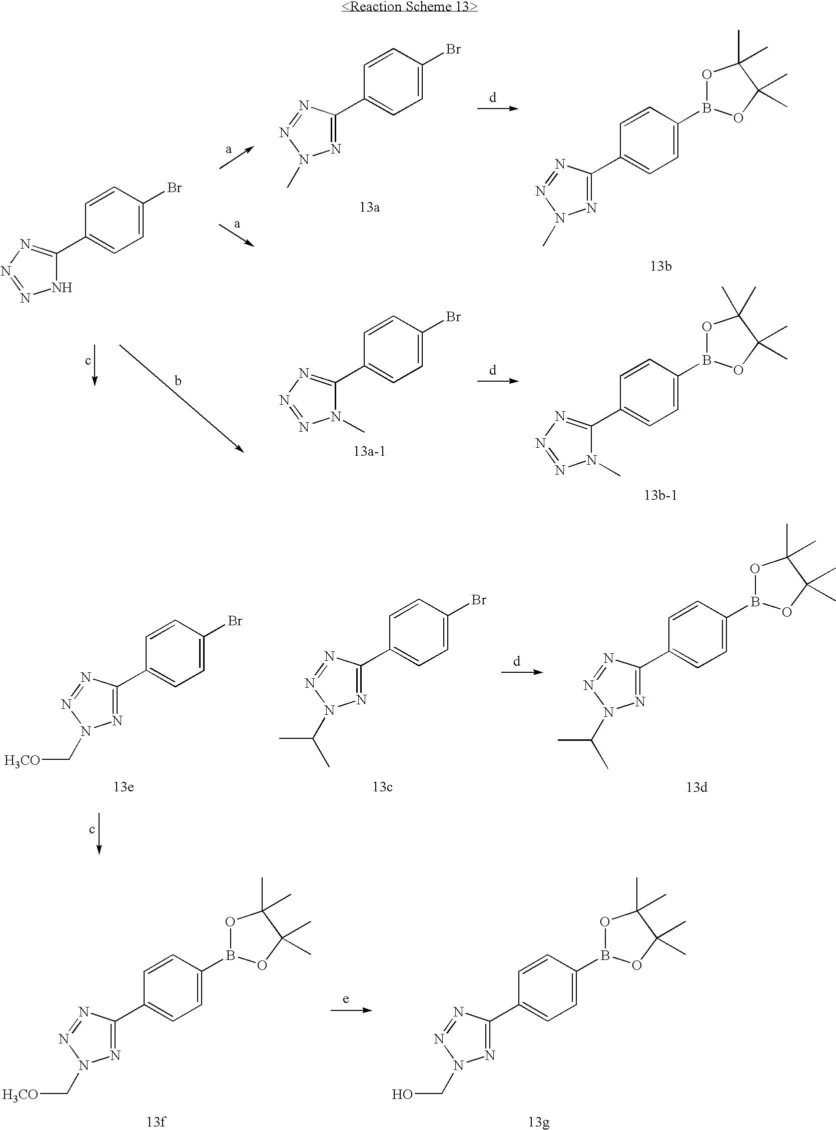

Reagents and Reaction Conditions:

a) Methyl iodide (CH₃I), 60% sodium hydride, N,N-dimethylformamide (DMF), room temperature, 4 hours.

b) Isopropyl bromide, 60% sodium hydride, N,N-dimethylformamide (DMF), room temperature, 4 hours.

c) Bromomethyl methyl ether, sodium hydroxide (NaOH), N,N-dimethylformamide (DMF), 0° C., 4 hours.

d) Bis(pinacolato)diboron, [1,1′-bis(diphenylphosphino)ferrocene]dichloropalladium (II) complex with dichloromethane (1:1), potassium acetate, dioxane, 90° C., 2 hours.

e) BBR₃ (tribromoborane), methylene chloride, room temperature, 5 hours.

Reaction Scheme 14 below illustrates synthesis of N-methyl-N-(4-(4,4,5,5-tetramethyl-1,3,2-dioxaborolan-2-yl)phenyl)acetamide among compounds of formula 11.

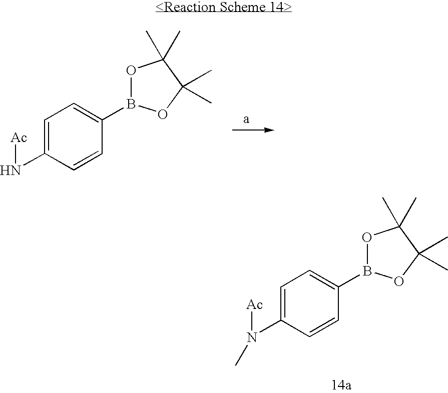

Reagents and Reaction Conditions:

a) Methyl iodide, 60% sodium hydride, N,N-dimethylformamide (DMF), room temperature, 1 hour.

Reaction Scheme 15 below illustrates synthesis of 2-methyl-6-(4-(4,4,5,5-tetramethyl-1,3,2-dioxaborolan-2-yl)phenyl)-4,5-dihydropyridazin-3(2H)-one among compounds of formula 11.

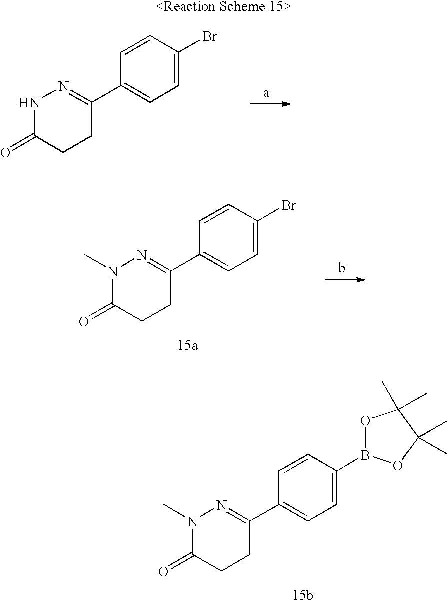

Reagents and Reaction Conditions:

a) Methyl iodide, triethylamine (Et₃N), tetrahydrofuran (THF), room temperature, 3 hours.

b) Bis(pinacolato)diboron, [1,1′-bis(diphenylphosphino)ferrocene]dichloropalladium (II) complex with dichloromethane (1:1), potassium acetate, dioxane, 90° C., 2 hours.

Reaction Scheme 16 below illustrates synthesis of 5-methyl-3-(4-(4,4,5,5-tetramethyl-1,3,2-dioxaborolan-2-yl)phenyl)1,2,4-oxadiazole among compounds of formula 11.

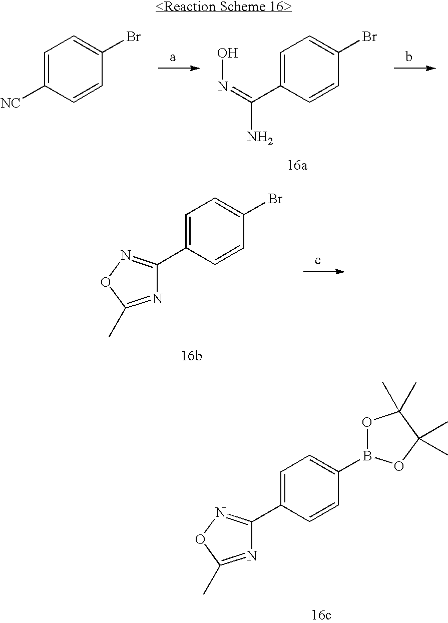

Reagents and Reaction Conditions:

a) Hydroxylamine, sodium bicarbonate, ethanol, 90° C., 3 hours.

b) N,N-dimethylacetamide dimethyl acetal, 1,4-dioxane, reflux, 12 hours.

c) Bis(pinacolato)diboron, [1,1′-bis(diphenylphosphino)ferrocene]dichloropalladium (II) complex with dichloromethane (1:1), potassium acetate, dioxane, 90° C., 2 hours.

Reaction Scheme 17 below illustrates synthesis of 2-methyl-5-(4-(4,4,5,5-tetramethyl-1,3,2-dioxaborolan-2-yl)phenyl)-1,3,4-oxadiazole among compounds of formula 11.

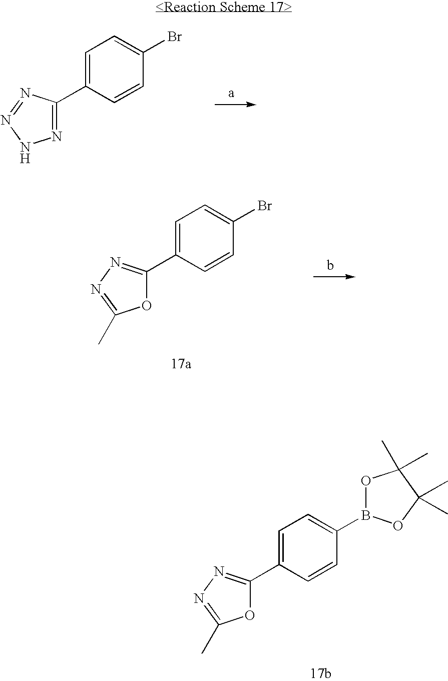

Reagents and Reaction Conditions:

a) Acetic anhydride, pyridine, reflux, 2 hours.

b) Bis(pinacolato)diboron, [1,1′-bis(diphenylphosphino)ferrocene]dichloropalladium (II) complex with dichloromethane (1:1), potassium acetate, dioxane, 90° C., 2 hours.

Reaction Scheme 18 below illustrates synthesis of 2-(4-(4,4,5,5-tetramethyl-1,3,2-dioxaborolan-2-yl)phenyl)-5-(trifluoromethyl)-1,3,4-oxadiazole among compounds of formula 11.

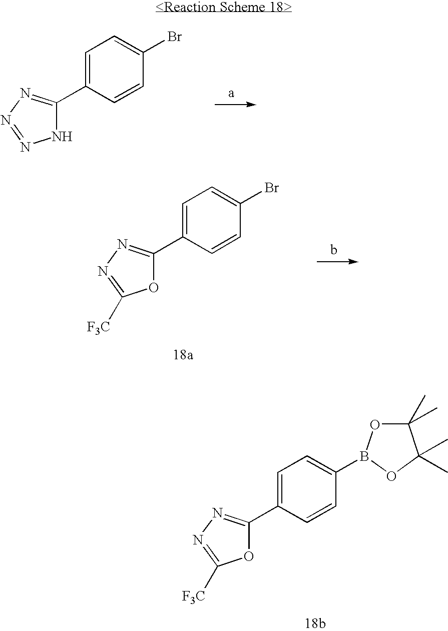

Reagents and Reaction Conditions:

a) Trifluoroacetic anhydride, pyridine, reflux.

b) Bis(pinacolato)diboron, [1,1′-bis(diphenylphosphino)ferrocene]dichloropalladium (II) complex with dichloromethane (1:1), potassium acetate (KOAc), N,N-dimethylformamide (DMF), 120° C., 2 hours.

Reaction Scheme 19 below illustrates synthesis of 2-(4-(4,4,5,5-tetramethyl-1,3,2-dioxaborolan-2-yl)phenyl)-1,3,4-oxadiazole among compounds of formula 11.

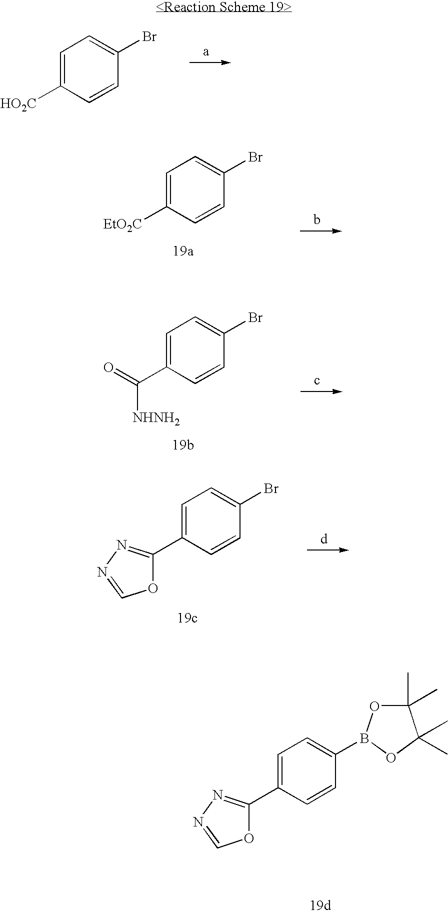

Reagents and Reaction Conditions:

a) Sulfuric acid, ethanol, 100° C., 6 hours.

b) Hydrazine, ethanol, reflux, 12 hours.

c) Acetic anhydride (AC₂O), 1-4 dioxane, reflux, 4 hours.

d) Bis(pinacolato)diboron), [1,1′-bis(diphenylphosphino)ferrocene]dichloropalladium (II) complex with dichloromethane (1:1), potassium acetate, dioxane, 90° C., 2 hours.

Reaction Scheme 20 below illustrates synthesis of 4,5-dimethyl-2-(4-(4,4,5,5-tetramethyl-1,3,2-dioxaborolan-2-yl)phenyl)oxazole among compounds of formula 11.

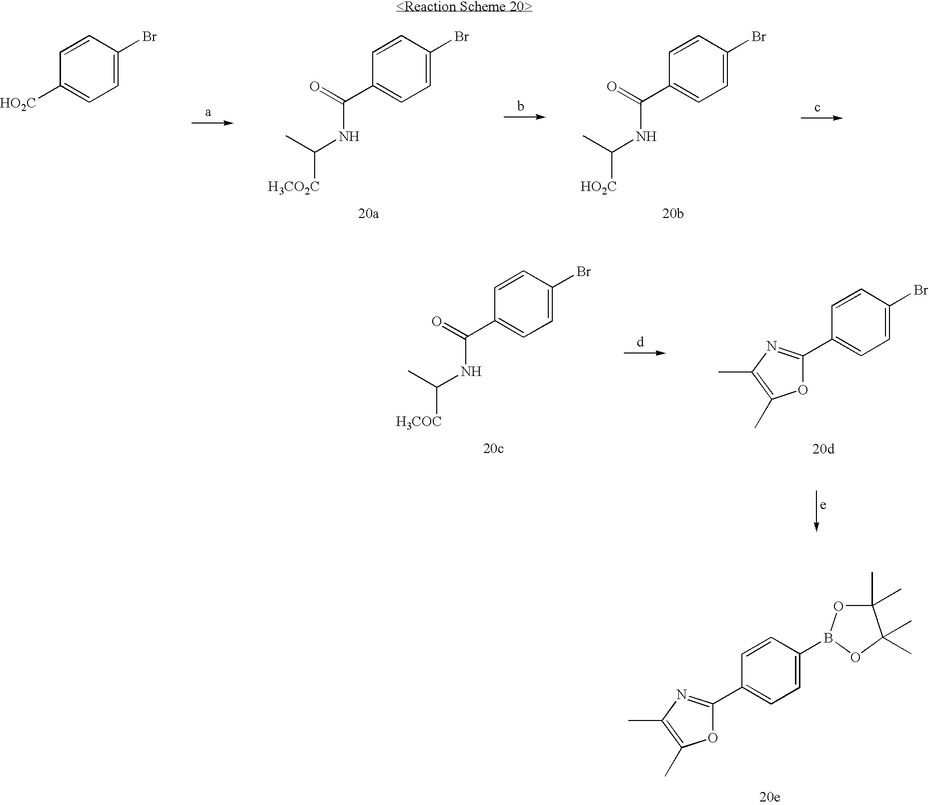

Reagents and Reaction Conditions:

a) Triethylamine (Et₃N), alanine methyl ester, ethyl chloroformate, tetrahydrofuran (THF), methanol, room temperature, 4 hours.

b) 2N—NaOH, methanol, reflux, 2 hours.

c) Acetic anhydride (AC₂O), pyridine, 90° C., 3 hours.

d) Sulfuric acid, acetic anhydride (AC₂O), 90° C., 1.5 hours.

e) Bis(pinacolato)diboron, [1,1′-bis(diphenylphosphino)ferrocene]dichloropalladium (II) complex with dichloromethane (1:1), potassium acetate, dioxane, 90° C., 2 hours.

Reaction Scheme 21 below illustrates synthesis of 1,3-dimethyl-5-(4-(4,4,5,5-tetramethyl-1,3,2-dioxaborolan-2-yl)phenyl-1H-pyrazole among compounds of formula 11.

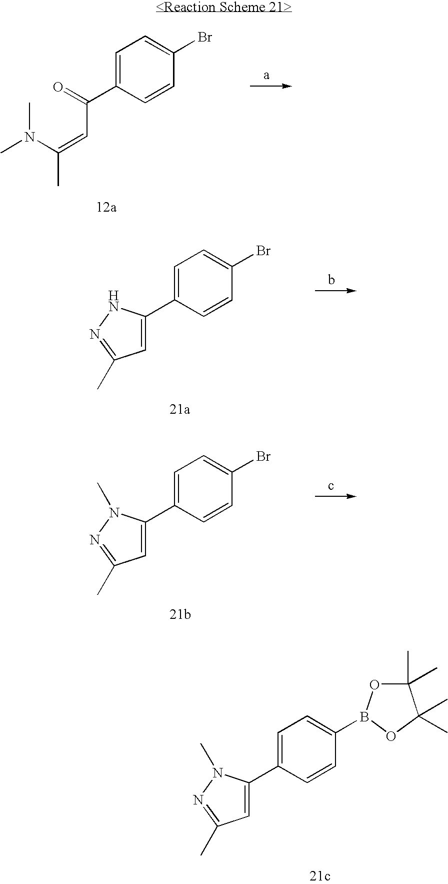

Reagents and Reaction Conditions:

a) Hydrazine, ethanol, 90° C., 6 hours.

b) Methyl iodide, N,N-dimethylformamide (DMF), 60% sodium hydride, room temperature, 1 hour.

c) Bis(pinacolato)diboron, [1,1′-bis(diphenylphosphino)ferrocene]dichloropalladium (II) complex with dichloromethane (1:1), potassium acetate, dioxane, 90° C., 2 hours.

Specifically, Reaction Scheme 22 below illustrates synthesis of compounds shown in the following Examples. R₁, R₄, and n are as defined in formula 1.

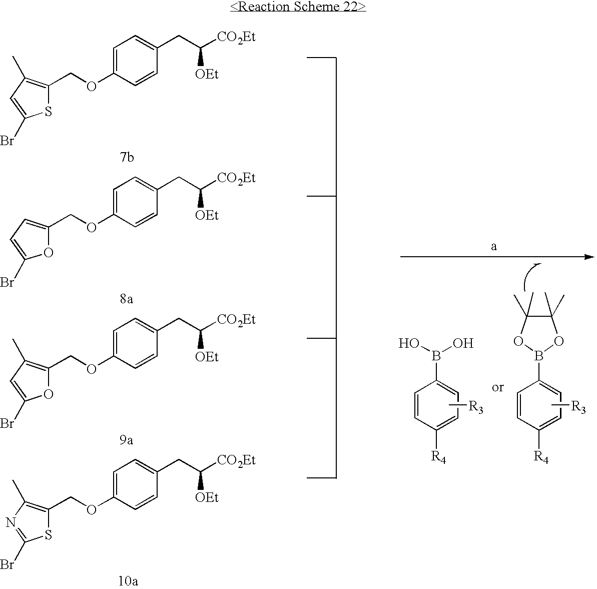

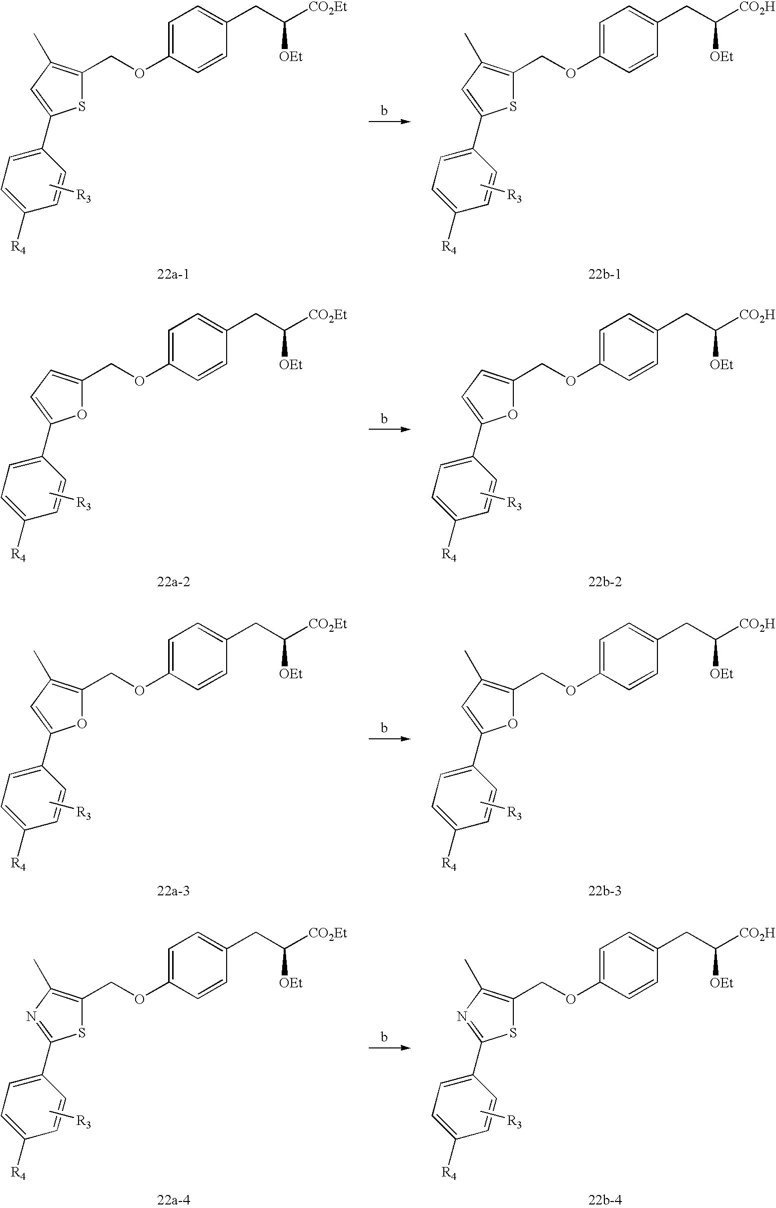

As shown in Reaction Scheme 22, each of brominated compounds (7b, 8a, 9a, and 10a) as a starting material was reacted with a boron compound as defined in formula 11 in the presence of a palladium catalyst to thereby form a carbon-carbon bond, and the resulting reaction product was hydrolyzed under basic conditions to afford a desired compound.

Reagents and Reaction Conditions:

a) Tetrakis(triphenylphosphine)palladium, cesium carbonate, dioxane, 90° C., 2 hours. Alternatively, tetrakis(triphenylphosphine)palladium, aq. potassium carbonate, toluene, ethanol, 90° C., 1 hour.

b) 1N—NaOH, ethanol, tetrahydrofuran (THF), 50° C., 1 hour.

Reaction Scheme 23 below illustrates a method for preparation of an alkali metal salt from the acid compound of Reaction Scheme 21.

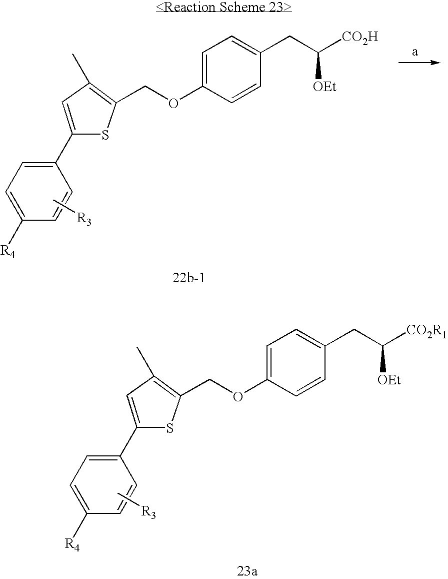

Reagents and Reaction Conditions:

a) 2-ethylhexanoic acid lithium salt, or 2-ethylhexanoic acid sodium salt, or 2-ethylhexanoic acid potassium salt, ethyl acetate/acetone, room temperature, 1 hour.

In Reaction Scheme 23, R₁ is an alkali metal, specifically lithium, sodium, or potassium.

Analysis of the compounds in accordance with the present invention was carried out by ¹H NMR spectra using Brucker DPX 400 MHz spectrometer and Agilent 1100 series LC/Mass.

Further, the present invention provides a pharmaceutical composition for modulation of peroxisome proliferator-activated receptor gamma (PPAR-γ), comprising a compound represented by formula 1, an optical isomer thereof or a pharmaceutically acceptable salt thereof, as an active ingredient.

Further, the present invention provides a use of the aforesaid pharmaceutical composition for modulation of peroxisome proliferator-activated receptor gamma (PPAR-γ), and a method for modulation of peroxisome proliferator-activated receptor gamma (PPAR-γ), comprising administering the aforesaid pharmaceutical composition to a subject.

When an EC₅₀ value of a compound of formula 1 on PPAR-γ activity was assayed, the compound of the present invention was confirmed to have EC₅₀ of 400 to 6000 nM for human PPAR-α and EC₅₀ of 7 to 1000 nM for human PPAR-γ. Therefore, a pharmaceutical composition comprising a compound of formula 1 in accordance with the present invention will be effective as a PPAR agonist that exhibits hypoglycemic, hypolipidemic and insulin resistance-reducing effects while alleviating adverse side effects. That is, a compound of formula 1 has hypoglycemic, hypolipidemic and insulin resistance-reducing effects on PPAR-mediated diseases or disorders, so it can be prophylactically or therapeutically effective for symptoms of PPAR-related diseases and conditions, such as obesity, diabetes, metabolic syndrome, hypertension, and hyperlipidemia.

Therefore, the present invention provides a use of the aforesaid composition for prevention or treatment of PPAR-mediated diseases (including obesity, diabetes, metabolic syndrome, hypertension and hyperlipidemia), and a method for prevention or treatment of PPAR-mediated diseases (including obesity, diabetes, metabolic syndrome, hypertension and hyperlipidemia), comprising administering the aforesaid composition to a subject.

Dosage forms of the composition of the present invention may include oral formulations such as powders, granules, tablets, capsules, suspensions, emulsions, syrups and aerosols, and parenteral formulations such as external preparations, suppositories, and sterile injections. That is, the composition may be formulated into a desired dosage form, depending upon diseases to be treated and ingredients, using any appropriate method known in the art, as disclosed in “Remington's Pharmaceutical Sciences,” Mack Publishing Co., Easton, Pa.

Depending upon desired applications, the pharmaceutical composition of the present invention can be administered via a conventional route, for example orally, intradermally, subcutaneously, intravenously, intramuscularly, rectally, intraorally, intranasally, intraocularly, etc. The pharmaceutical composition may further comprise one or more pharmaceutically acceptable additives such as excipients, disintegrating agents, sweeteners, binders, coating agents, blowing agents, lubricants, glidants, solubilizers, etc, depending upon dosage forms of the composition.

The compound of formula 1 may be administered at a dose of 0.1 mg to 1000 mg/kg BW once or several times a day. As will be apparent to those skilled in the art, the effective dose of the active compound may vary depending upon various factors such as particular factors of patients, co-administered drugs, and severity of diseases.

### ADVANTAGEOUS EFFECTS

As discussed hereinbefore, the present invention provides a novel phenylpropionic acid derivative of formula 1 and a method for preparing the same.

Further, the compound of the present invention has modulatory activity on peroxisome proliferator-activated receptor gamma (PPAR-γ) and therefore exhibits hypoglycemic, hypolipidemic and insulin resistance-reducing effects on PPAR-mediated diseases or disorders. As a result, the compound of formula 1 can be effective for prevention or treatment of PPAR-related diseases such as obesity, diabetes, hypertension, hypertriglyceridemia, etc.

### DESCRIPTION OF THE DRAWINGS

FIG. 1 shows test results for binding capacity of inventive compounds with Trap220 which is a main cofactor implicated in adipogenic differentiation.

### MODE FOR INVENTION

Now, the present invention will be described in more detail with reference to the following Examples, Preparation Examples and Experimental Examples. Preparation Examples illustrate synthesis of intermediates produced during preparation of compounds in accordance with the present invention. These examples are provided only for illustrating the present invention and should not be construed as limiting the scope and spirit of the present invention.

### Preparation Example 1

### Preparation of (2S)-2-ethoxy-3-(4-hydroxyphenyl)-propionic acid ethyl ester (2d)

### Step 1: Preparation of 3-(4-(benzyloxy)phenyl)-2-ethoxy acrylic acid ethyl ester (2a)

Potassium t-butoxide (t-BuOK, 13 g) and triethyl 2-ethoxyphosphonoacetate (25 g, 93.19 mmol) were added to toluene (150 mL) under a nitrogen atmosphere, and 4-benzyloxy benzaldehyde (10 g, 47.12 mmol) was added dropwise thereto at room temperature over 10 min. The reactants were stirred at room temperature for 40 min, and the solution was adjusted to pH of 2 to 3 with addition of 2N—HCl, followed by extraction with ethyl acetate (300 mL). The organic layer was washed with water (50 mL×2) and brine (30 μL). The organic layer was separated, dried over anhydrous magnesium sulfate, and filtered under reduced pressure to remove an organic solvent. The residue was crystallized from ethanol at 5° C. to give the title compound 3-(4-(benzyloxy)phenyl)-2-ethoxy acrylic acid ethyl ester (2a). Yield=72%.

¹H NMR (CDCl₃, 400 MHz): 7.74-7.76 (d, 2H, J=8.8 Hz), 7.43-7.39 (m, 5H), 7.03-7.01 (d, 2H, J=8.8 Hz), 6.88 (s, 1H), 5.12 (s, 2H), 4.20 (q, 2H, J=7.2 Hz), 3.91 (q, 2H, J=7.0 Hz), 2.49 (m, 2H), and 1.26 (t, 2H, J=7.2 Hz)

### Step 2: Preparation of 2-ethoxy-3-(4-hydroxyphenyl)-propionic acid ethyl ester (2b)

3-(4-(benzyloxy)phenyl)-2-ethoxy acrylic acid ethyl ester (2a, 8.0 g, 24.53 mmol) obtained in Step 1 was subjected to hydrogenation using 10% Pd/C to give 2-ethoxy-3-(4-hydroxyphenyl)-propionic acid ethyl ester (2b) as a colorless oil. Yield: 91%.

¹H NMR (CDCl₃, 400 MHz): 7.09 (d, 2H, J=8.6 Hz), 6.73 (d, 2H, J=8.8 Hz), 5.49 (s, 2H), 4.21 (q, 2H, J=7.2 Hz), 3.96 (t, 2H, J=6.9 Hz), 3.58 (m, 1H), 3.34 (m, 1H), 2.92 (d, 2H, J=6.4 Hz), 1.26 (t, 3H, J=7.2 Hz), and 1.14 (t, 3H, J=7.2 Hz)

### Step 3: Preparation of (2S)-2-ethoxy-3-(4-hydroxyphenyl)-propionic acid (2c)

2-ethoxy-3-(4-hydroxyphenyl)-propionic acid ethyl ester (2b, 13.7 g, 57.46 mmol) obtained in Step 2 was dissolved in 0.1M phosphate buffer (pH=7, 100 mL), to which Viscozyme-L (42 mL) was then added. The reaction mixture was stirred at 25° C. for 48 hours, and the reaction solvent was removed under reduced pressure. Methanol (70 mL) was added to the residue and the resulting mixture was stirred for 30 min, followed by filtration. Methanol was removed under reduced pressure, and unreacted ester was removed using water and t-BME. The solution was adjusted to pH of 2 to 3 with addition of 6N HCl, followed by extraction with t-BME two times. The organic solvent was evaporated to give the title compound (2S)-2-ethoxy-3-(4-hydroxyphenyl)-propionic acid (2c). Yield: 30%. Optical activity of the compound was assayed by determining optical purity (enantiomeric excess) of the compound using the following column. The optical purity as measured was 99.54%.

Column: Shiseido Capcell Pak C18 MG 3.0×250 mm, 5 μm

Mobile phase: MeOH/H₂O=8/2, 0.1%-TEA, 0.05%-H₃PO₄.

Flow rate: 0.5 mL/min

[α]^{D}=−33.1

¹H NMR (DMSO-d₆, 400 MHz): 12.08 (bs, 1H). 7.01 (d, 2H, J=8.6 Hz), 6.65 (d, 2H, J=8.4 Hz), 3.87 (2q, 1H, J=5.3, 7.7 Hz), 3.51-3.46 (m, 1H), 3.29 (m, 1H), 2.95 (m, 1H), 2.80 (m, 1H), and 1.11 (t, 3H, J=7.2 Hz).

### Step 4: Preparation of (2S)-2-ethoxy-3-(4-hydroxyphenyl)-propionic acid ethyl ester (2d)

(2S)-2-ethoxy-3-(4-hydroxyphenyl)-propionic acid (2c, 3.02 g, 14.36 mmol) prepared in Step 3 was dissolved in ethanol (20 mL), to which thionyl chloride (SOCl₂, 1.2 mL) was then added, followed by reflux for 3 hours. After completion of the reaction was confirmed by thin layer chromatography (TLC), the solvent was removed under reduced pressure, followed by extraction with water (100 mL) and ethyl acetate (100 mL). An organic layer was washed with water (50 mL×2) and brine (30 mL). The organic layer was separated, dried over anhydrous magnesium sulfate, and filtered under reduced pressure to remove ethyl acetate. The residue was solidified with ethyl acetate and hexane to give the title compound (2S)-2-ethoxy-3-(4-hydroxyphenyl)-propionic acid ethyl ester (2d) as white solids. Yield: 93%.

[α]^{D}=−18.7

¹H NMR (CDCl₃, 400 MHz): 7.09 (d, 2H, J=8.6 Hz), 6.73 (d, 2H, J=8.8 Hz), 5.49 (s, 2H), 4.18 (q, 2H, J=7.2 Hz), 3.97 (t, 2H, J=6.9 Hz), 3.61-3.58 (2q, 1H, J=7.0 Hz), 3.37-3.34 (2q, 1H, J=7.0 Hz), 2.94 (d, 2H, J=6.4 Hz), 1.26 (t, 3H, J=7.2 Hz), and 1.14 (t, 3H, J=7.2 Hz)

### Preparation Example 2

### Preparation of (S)-3-(4-((5-bromo-3-methylthiophen-2-yl)methoxy)phenyl)-2-ethoxypropionic acid ethyl ester (7b)

### Step 1: Preparation of (3-methylthiophen-2-yl)methanol (3a)

As shown in Reaction Scheme 3, 3-methyl-2-thiophene-carboxaldehyde (40 g, 317 mmol) was dissolved in ethanol (500 mL) at 0° C., and sodium borohydride (22 g, 581 mmol) was then gradually added thereto. The solution was warmed to room temperature, followed by reaction for 1 hour. After completion of the reaction was confirmed by TLC, unreacted sodium borohydride was inactivated using water and aqueous ammonium chloride, followed by ethyl acetate extraction. An organic layer was washed with water (200 mL×2). The organic layer was separated, dried over anhydrous magnesium sulfate, and filtered under reduced pressure to remove the solvent, thus affording the title compound 3-methylthiophen-2-yl)methanol (3a). Yield: 95%.

¹H NMR (CDCl₃, 400 MHz): 7.14 (d, 2H, J=8.6 Hz), 6.82 (d, 1H, J=5.2 Hz), 4.74 (s, 2H), and 2.22 (s, 3H).

### Step 2: Preparation of (S)-2-ethoxy-3-(4-((methylthiophen-2-yl)methoxy)phenyl propionic acid ethyl ester (7a)

(2S)-2-ethoxy-3-(4-hydroxyphenyl)-butyric acid ethyl ester (21 g, 88.13 mmol) from Preparation Example 1, (3-methylthiophen-2-yl)methanol (11 g, 85.11 mmol) from Step 1 of Preparation Example 2, and triphenylphosphine (29 g, 110.56 mmol) were dissolved in dichloromethane (500 mL). The resulting reaction solution was cooled to 0° C., and diisopropyl azodicarboxylate (21 g, 103.85 mmol) was then gradually added thereto. The solution was warmed to room temperature, followed by reaction for 2 hours. After completion of the reaction was confirmed by TLC, the solvent was removed under reduced pressure and triphenylphosphine oxide was then solidified using ethyl ether (100 mL) and hexane (500 mL), followed by filtration. The filtrate was concentrated and purified by silica gel column chromatography using ethyl acetate and hexane as a developing solvent, thus affording the title compound (S)-2-ethoxy-3-(4-((methylthiophen-2-yl)methoxy)phenylpropionic acid ethyl ester (7a). Yield: 65%.

¹H NMR (CDCl₃, 400 MHz): 7.19 (d, 1H, J=5.2 Hz), 7.14 (d, 2H, J=8.6 Hz), 6.89 (d, 2H, J=8.8 Hz), 6.83 (d, 1H, J=5.2 Hz), 5.07 (s, 2H), 4.15 (q, 2H, J=6.8 Hz), 3.95 (m, 1H), 3.58 (m, 1H), 3.34 (m, 1H), 2.94 (m, 2H), 2.22 (s, 3H), 1.23 (t, 3H, J=7.2 Hz), and 1.14 (t, 3H, J=7.2 Hz).

### Step 3: Preparation of (S)-3-(4-((5-bromo-3-methylthiophen-2-yl)methoxy)phenyl)-2-ethoxypropionic acid ethyl ester (7b)

(S)-2-ethoxy-3-(4-((methylthiophen-2-yl)methoxy)phenylpropionic acid ethyl ester (18 g, 51.66 mmol) and N-bromosuccinimide (9.4 g, 52.81 mmol) were dissolved in N,N-dimethylformamide (DMF, 100 mL), and the reactants were reacted at room temperature for 3 hours. After the reaction was confirmed by LC/Mass, aqueous sodium thiosulfate (200 mL) was added to the reactants, followed by extraction with ethyl acetate (300 mL). An organic layer was washed with water (100 mL×2) and brine (50 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound (S)-3-(4-((5-bromo-3-methylthiophen-2-yl)methoxy)phenyl)-2-ethoxypropionic acid ethyl ester (7b). Yield: 90%.

¹H NMR (CDCl₃, 400 MHz): 7.16 (d, 2H, J=8.4 Hz), 6.85 (d, 2H, J=8.8 Hz), 6.78 (s, 1H), 4.99 (s, 2H), 4.15 (q, 2H, J=6.8 Hz), 3.96 (m, 1H), 3.58 (m, 1H), 3.34 (m, 1H), 2.94 (m, 2H), 2.18 (s, 3H), 1.22 (t, 3H, J=7.2 Hz), and 1.15 (t, 3H, J=7.2 Hz).

### Preparation Example 3

### Preparation of (S)-3-(4-((5-bromofuran-2-yl)methoxy)phenyl)-2-ethoxypropionic acid ethyl ester (8a)

### Step 1: Preparation of (5-bromofuran-2-yl)methanol (4a)

Analogously to Step 1 of Preparation Example 1, 5-bromofurancarboxaldehyde (17.5 g, 100 mmol) was reacted with sodium borohydride to afford the title compound (5-bromofuran-2-yl)methanol (4a). Yield: 80%.

¹H NMR (CDCl₃, 400 MHz): 6.48 (d, 2H, J=3.8 Hz), 6.36 (m, 1H), and 4.65 (s, 2H).

### Step 2: Preparation of (S)-ethyl 3-(4-((5-bromofuran-2-yl)methoxy)phenyl)-2-ethoxypropanoate (8a)

Analogously to Step 2 of Preparation Example 1, the title compound (S)-ethyl 3-(4-((5-bromofuran-2-yl)methoxy)phenyl)-2-ethoxypropanoate (8a) was synthesized from Compound 4a and Compound 2d of Preparation Example 1 through the Mitsunobu reaction. Yield: 40%.

¹H NMR (CDCl₃, 400 MHz): 7.17 (d, 1H, J=5.2 Hz), 6.92 (d, 2H, J=8.6 Hz), 6.53 (m, 1H), 6.47 (m, 1H), 4.99 (s, 2H), 4.16 (q, 2H, J=6.8 Hz), 3.95 (m, 1H), 3.58 (m, 1H), 3.33 (m, 1H), 2.93 (m, 2H), 1.25 (t, 3H, J=7.2 Hz), and 1.14 (t, 3H, J=7.2 Hz).

### Preparation Example 4

### Preparation of (S)-ethyl-3-(4-((5-bromo-3-methylfuran-2-yl)methoxy)phenyl)-2-ethoxypropanoate (9a)

### Step 1: Preparation of (5-bromo-3-methylfuran-2-yl)methanol (5a)

Ethyl 5-bromo-3-methylfuran-2-carboxylate (4.7 g, 20.16 mmol) was dissolved in tetrahydrofuran (THF, 20 mL). 2 equivalents of lithium aluminum hydride (LAH) were gradually added to the solution while being maintained at 0° C., followed by reaction for 1 hour. The reaction was terminated with addition of 1N—NaOH and 1N—HCl solution, followed by extraction with ethyl acetate (100 mL). The organic layer was washed with water (100 mL×2) and brine (50 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound (5-bromo-3-methylfuran-2-yl)methanol (5a). Yield: 40%.

MS (ESI⁺) m/z 190.9 (M⁺¹)

### Step 2: Preparation of (S)-3-(4-((5-bromo-3-methylfuran-2-yl)methoxy)phenyl)-2-ethoxypropionic acid ethyl ester (9a)

Analogously to Step 2 of Preparation Example 1, the title compound (S)-3-(4-((5-bromo-3-methylfuran-2-yl)methoxy)phenyl)-2-ethoxypropionic acid ethyl ester (9a) was synthesized from Compounds 5a and 2d through the Mitsunobu reaction. Yield: 45%.

MS (ESI⁺) m/z 411.2 (M⁺¹)

### Preparation Example 5

### Preparation of (S)-ethyl 3-(4-((2-bromo-4-methylthiazol-2-yl)methoxy)phenyl)-2-ethoxypropanoate (10a)

### Step 1: Preparation of (2-bromo-4-methylthiazol-5-yl)methanol (6a)

Analogously to Step 1 of Preparation Example 4, the title compound (2-bromo-4-methylthiazol-5-yl)methanol (6a) was synthesized from ethyl 2-bromo-4-methylthiazole-5-carboxylate (5.0 g, 20.00 mmol).

MS (ESI⁺) m/z 207.9 (M⁺¹)

### Step 2: Preparation of (S)-ethyl 3-(4-((2-bromo-4-methylthiazol-2-yl)methoxy)phenyl)-2-ethoxypropanoate (10a)

Analogously to Step 2 of Preparation Example 1, the title compound (S)-ethyl 3-(4-((2-bromo-4-methylthiazol-2-yl)methoxy)phenyl)-2-ethoxypropanoate (10a) was synthesized from Compound 6a and Compound 2d of Preparation Example 1 through the Mitsunobu reaction. Yield: 35%.

MS (ESI⁺) m/z 428.2 (M⁺¹)

### Preparation Example 6

### Preparation of 5-tert-butyl-3-(4-(4,4,5,5-tetramethyl-1,3,2-dioxaborolan-2-yl)phenyl)isoxazole (11d)

### Step 1: Preparation of 4-bromobenzaldehyde oxime (11a)

4-bromobenzaldehyde (10 g, 54.05 mmol) and hydroxylamine (7.5 g, 107.9 mmol) were dissolved in pyridine (200 mL) at 0° C., and the solution was warmed to room temperature, followed by reaction for 2 hours. After completion of the reaction was confirmed by TLC, the reaction solution was adjusted to pH 5 with addition of concentrated hydrochloric acid (10 mL) and water (30 mL), and water (50 mL) was added to form solids. The resulting solids were filtered, washed with water (100 mL) and dried to give 4-bromobenzaldehyde oxime (11a). Yield: 95%.

¹H NMR (CDCl₃, 400 MHz): 8.07 (s, 1H), 7.50 (d, 2H, J=8.8 Hz), and 7.42 (d, 1H, J=8.8 Hz).

### Step 2: Preparation of 4-bromo-N-hydroxybenzimidoyl chloride (11b)

4-bromobenzaldehyde oxime (10.5 g, 52.5 mmol) and N-chlorosuccinimide (7.7 g, 57.66 mmol) were dissolved in N,N-dimethylformamide (60 mL), followed by reaction at room temperature for 1 hour. After completion of the reaction was confirmed by TLC, water (200 mL) was added to the reactants to result in solidification. The resulting solids were dried and recrystallized from diethyl ether and hexane to afford the title compound 4-bromo-N-hydroxybenzimidoyl chloride (11b). Yield: 80%.

MS (ESI⁺) m/z 233.9 (M⁺¹)

### Step 3: Preparation of 3-(4-bromophenyl)-5-tert-butylisoxazole (11c)

4-bromo-N-hydroxybenzimidoyl chloride (13 g, 55.44 mmol), 3,3-dimethyl-1-butyne (7.5 g, 71.29 mmol) and triethylamine (10 mL) were dissolved in dichloromethane (100 mL), followed by reaction at room temperature for 5 hours. After completion of the reaction was confirmed by TLC, solids produced in the reactants were filtered, and the filtrate was washed with 1N aqueous hydrochloric acid (30 mL) and water (50 mL). Then, the organic layer was separated, dried over anhydrous magnesium sulfate, and filtered under reduced pressure to remove the solvent. The residue was recrystallized from diethyl ether and hexane to afford the title compound 3-(4-bromophenyl)-5-tert-butylisoxazole (11c). Yield: 70%.

¹H NMR (CDCl₃, 400 MHz): 7.85 (d, 2H, J=8.0 Hz), 7.56 (d, 2H, J=8.0 Hz), 6.20 (s, 1H), and 1.37 (s, 9H).

### Step 4: Preparation of 5-tert-butyl-3-(4-(4,4,5,5-tetramethyl-1,3,2-dioxaborolan-2-yl)phenyl)isoxazole (11d)

3-(4-bromophenyl)-5-tert-butylisoxazole (8.8 g, 31.2 mmol), bis(pinacolato)diboron (9.5 g, 37.41 mmol), bis(diphenylphosphino)ferrocene dichloropalladium (1.27 g, 1.56 mmol), and potassium acetate (8.8 g, 31.2 mmol) were added to dioxane (100 mL), followed by reaction at 90° C. for 2 hours. After completion of the reaction was confirmed by TLC, the reactants were filtered through celite. The filtrate was extracted with water (200 mL) and ethyl acetate (500 mL). The organic layer was washed with water (100 mL×2) and brine (50 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-tert-butyl-3-(4-(4,4,5,5-tetramethyl-1,3,2-dioxaborolan-2-yl)phenyl)isoxazole (11d). Yield: 90%.

¹H NMR (CDCl₃, 400 MHz): 7.85 (d, 2H, J=8.0 Hz), 7.77 (d, 2H, J=8.0 Hz), 6.26 (s, 1H), 1.42, 1.35 (each s, 12H), and 1.30 (s, 9H).

### Preparation Example 7

### Preparation of (3-(4-(4,4,5,5-tetramethyl-1,3,2-dioxaborolan-2-yl)phenyl)isoxazol-5-yl)methanol (11h)

### Step 1: Preparation of (3-(4-bromophenyl)isoxazol-5-yl)methanol (11e)

4-bromo-N-hydroxybenzimidoyl chloride (3.5 g, 14.9 mmol) synthesized in Step 2 of Preparation Example 6, propargyl alcohol (2.74 mL, 45 mmol) and triethylamine (Et₃N, 7.7 mL) were added to methylene chloride (50 mL) and the reactants were stirred at room temperature for 1.5 hours. After completion of the reaction was confirmed by TLC, extraction was done with water (200 mL) and methylene chloride (500 mL). The organic layer was washed with water (100 mL×2) and brine (50 mL). The organic layer was separated, dried over anhydrous magnesium sulfate, and filtered under reduced pressure to remove the solvent. The residue was purified by silica gel column chromatography using ethyl acetate and hexane as a developing solvent, thus affording the title compound (3-(4-bromophenyl)isoxazol-5-yl)methanol (11e). Yield: 69%.

¹H NMR (CDCl₃, 400 MHz): 7.66 (d, 2H, J=2.4 Hz), 7.64 (d, 2H, J=1.6 Hz), 6.53 (s, 1H), and 4.81 (s, 2H).

### Step 2: Preparation of (3-(4-(4,4,5,5-tetramethyl-1,3,2-dioxaborolan-2-yl)phenyl)isoxazol-5-yl)methanol (11h)

(3-(4-bromophenyl)isoxazol-5-yl)methanol (300 mg, 1.18 mmol), bis(pinacolato)diboron (750 mg, 3 mmol), bis(diphenylphosphino)ferrocene dichloropalladium (193 mg, 0.24 mmol), and potassium acetate (348 mg, 3.54 mmol) were added to N,N-dimethylformamide (4 mL), followed by reaction at 90° C. for 2 hours. After completion of the reaction was confirmed by TLC, the reactants were filtered through celite. The filtrate was extracted with water (20 mL) and ethyl acetate (50 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound (3-(4-(4,4,5,5-tetramethyl-1,3,2-dioxaborolan-2-yl)phenyl)isoxazol-5-yl)methanol (11h). Yield: 60%.

¹H NMR (CDCl₃, 400 MHz): 7.88 (d, 2H, J=8.0 Hz), 7.79 (d, 2H, J=7.6 Hz), 6.58 (s, 1H), 4.81 (s, 2H), and 1.25 (s, 12H).

### Preparation Example 8

### Preparation of 5-(methoxymethyl)-3-(4-(4,4,5,5-tetramethyl-1,3,2-dioxaborolan-2-yl)phenyl)isoxazole (11g)

### Step 1: Preparation of 3-(4-bromophenyl)-5-(methoxymethyl)isoxazole (11f)

3-(4-bromophenyl)isoxazol-5-yl)methanol (5.0 g, 19.7 mmol) synthesized in Step 1 of Preparation Example 7, and 60% sodium hydride (1 g) were added to N,N-dimethylformamide (50 mL) and the mixture was stirred for 15 min. After methyl iodide was added thereto and completion of the reaction was confirmed by TLC, extraction was carried out with water (20 mL) and ethyl acetate (100 mL). The organic layer was washed with water (50 mL×2) and brine (20 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 3-(4-bromophenyl)-5-(methoxymethyl)isoxazole (11f). Yield: 95%.

¹H NMR (CDCl₃, 400 MHz): 7.67 (d, 2H, J=7.2 Hz), 7.58 (d, 2H, J=7.6 Hz), 6.53 (s, 1H), 4.57 (s, 2H), and 3.45 (s, 3H).

### Step 2: Preparation of 5-(methoxymethyl)-3-(4-(4,4,5,5-tetramethyl-1,3,2-dioxaborolan-2-yl)phenyl)isoxazole (11g)

To N,N-dimethylformamide (7 mL) were added 3-(4-bromophenyl)-5-(methoxymethyl)isoxazole (526 mg, 1.96 mmol), bis(pinacolato)diboron (1.25 g, 4.9 mmol), bis(diphenylphosphino)ferrocene dichloropalladium (320 mg, 0.39 mmol) and potassium acetate (577 mg, 6 mmol), followed by reaction at 90° C. for 2 hours. After completion of the reaction was confirmed by TLC, reactants were filtered through celite. The filtrate was extracted with water (30 mL) and ethyl acetate (30 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(methoxymethyl)-3-(4-(4,4,5,5-tetramethyl-1,3,2-dioxaborolan-2-yl)phenyl)isoxazole (11g). Yield: 80%.

¹H NMR (CDCl₃, 400 MHz): 7.88 (d, 2H, J=8.4 Hz), 7.79 (d, 2H, J=8.0 Hz), 6.58 (s, 1H), 4.57 (s, 2H), 3.45 (s, 3H), and 1.33 (s, 12H).

### Preparation Example 9

### Preparation of 3-methyl-5-(4-(4,4,5,5-tetramethyl-1,3,2-dioxaborolan-2-yl)phenyl)isoxazole (12c)

### Step 1: Preparation of 1-(4-bromophenyl)-3-(dimethylamino)but-2-en-1-one (12a)

4-bromoacetophenone (3.55 g, 17.84 mmol) and N,N-dimethylacetamide dimethyl acetal (DMA acetal, 8.9 mL, 62.44 mmol) were dissolved in dioxane (50 mL), followed by reflux for 12 hours. After completion of the reaction was confirmed by TLC, water (150 mL) and ethyl acetate (300 mL) were added to the reactants, followed by extraction. The organic layer was washed with brine. The organic layer was separated, dried over anhydrous magnesium sulfate, and filtered under reduced pressure to remove ethyl acetate. The resulting solids were dried and recrystallized from hexane to afford the title compound 1-(4-bromophenyl)-3-(dimethylamino)but-2-en-1-one (12a). Yield: 65%.

¹H NMR (CDCl₃, 400 MHz): 7.69 (d, 2H, J=8.4 Hz), 7.46 (d, 2H, J=8.4 Hz), 5.58 (s, 1H), 3.06 (s, 6H), and 2.63 (s, 3H).

### Step 2: Preparation of 5-(4-bromophenyl)-3-methylisoxazole (12b)

1-(4-bromophenyl)-3-(dimethylamino)but-2-en-1-one (2.68 g, 10 mmol) synthesized in Step 1 of Preparation Example 9, and ammonium hydroxide (3 eq.) were dissolved in ethanol (50 mL) and the solution was warmed to 90° C., followed by reaction for 3 hours. After completion of the reaction was confirmed by TLC, extraction was carried out with water (100 mL) and ethyl acetate (250 mL). The organic layer was washed with brine, dried over anhydrous magnesium sulfate, and filtered under reduced pressure to remove the solvent. The resulting solids were washed with hexane, filtered and dried under vacuum to afford the title compound 5-(4-bromophenyl)-3-methylisoxazole (12b). Yield: 90%.

¹H NMR (CDCl₃, 400 MHz): 7.51 (m, 4H), 6.35 (s, 1H), and 2.34 (s, 3H).

### Step 3: Preparation of 3-methyl-5-(4-(4,4,5,5-tetramethyl-1,3,2-dioxaborolan-2-yl)phenyl)isoxazole (12c)

To N,N-dimethylformamide (DMF, 30 mL) were added 5-(4-bromophenyl)-3-methylisoxazole (2.5 g, 10.45 mmol), bis(pinacolato)diboron (5.0 g, 19.69 mmol), bis(diphenylphosphino)ferrocene dichloropalladium (900 mg, 1.1 mmol), and potassium acetate (3 g, 30.56 mmol), followed by reaction at 90° C. for 2 hours. After completion of the reaction was confirmed by TLC, reactants were filtered through celite. The filtrate was extracted with water (10 mL) and ethyl acetate (10 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 3-methyl-5-(4-(4,4,5,5-tetramethyl-1,3,2-dioxaborolan-2-yl)phenyl)isoxazole (12c). Yield: 70%.

¹H NMR (CDCl₃, 400 MHz): 7.86 (d, 2H, J=8.4 Hz), 7.72 (d, 2H, J=8.4 Hz), 6.39 (s, 1H), 2.34 (s, 2H), and 1.35 (s, 12H).

### Preparation Example 10

### Preparation of ethyl 2-oxo-2-(5-(4-(4,4,5,5-tetramethyl-1,3,2-dioxaborolan-2-yl)phenyl)isoxazol-3-yl)acetate (12h)

### Step 1: Preparation of ethyl 4-(4-bromophenyl)-4-hydroxy-2-oxobut-3-enoate (12d)

4-bromoacetophenone (5.5 g, 27.64 mmol), diethyl oxalate (6.1 mL, 41.5 mmol) and 60% sodium hydride (2.2 g) were dissolved in toluene (80 mL), followed by reaction under reflux for 1 hour. After completion of the reaction was confirmed by TLC, water (100 mL) and ethyl acetate (250 mL) were added to the reactants, followed by extraction. The organic layer was washed with brine and distilled under reduced pressure. The residue was purified by silica gel column chromatography using ethyl acetate and n-hexane as a developing solvent, thus affording the title compound ethyl 4-(4-bromophenyl)-4-hydroxy-2-oxobut-3-enoate (12d). Yield: 80%.

¹H NMR (CDCl₃, 400 MHz): 7.85 (d, 2H, J=8.4 Hz), 7.64 (d, 2H, J=8.4 Hz), 7.01 (s, 1H), 4.39 (q, 2H, J=7.2 Hz), and 1.41 (t, 3H, J=6.8 Hz).

### Step 2: Preparation of ethyl 5-(4-bromophenyl)isoxazole-3-carboxylate (12e)

Ethyl 4-(4-bromophenyl)-4-hydroxy-2-oxobut-3-enoate (2.4 g, 8.76 mmol) was completely dissolved in ethanol (50 mL) to which NH₂OH.HCl (3 eq.) was then added, followed by reaction under reflux for 2 hours. After completion of the reaction was confirmed by TLC, water (100 mL) and ethyl acetate (250 mL) were added to the reactants, followed by extraction. The organic layer was washed with brine and distilled under reduced pressure. The resulting solids were washed with hexane, filtered and dried under vacuum to afford the title compound ethyl 5-(4-bromophenyl)isoxazole-3-carboxylate (12e).

¹H NMR (CDCl₃, 400 MHz): 7.67 (m, 4H), 6.91 (s, 1H), 4.46 (q, 2H, J=6.8 Hz), and 1.44 (t, 3H, J=7.6 Hz).

### Step 3: Preparation of ethyl 2-oxo-2-(5-(4-(4,4,5,5-tetramethyl-1,3,2-dioxaborolan-2-yl)phenyl)isoxazol-3-yl)acetate (12h)

Analogously to Step 4 of Preparation Example 6, ethyl 2-oxo-2-(5-(4-(4,4,5,5-tetramethyl-1,3,2-dioxaborolan-2-yl)phenyl)isoxazol-3-yl)acetate (12h) was prepared from ethyl 5-(4-bromophenyl)isoxazole-3-carboxylate (2.0 g, 6.75 mmol). Yield: 60%.

¹H NMR (CDCl₃, 400 MHz): 7.71 (d, 2H, J=8.4 Hz), 7.59 (d, 2H, J=8.4 Hz), 6.88 (s, 1H), 4.45 (q, 2H, J=6.8 Hz), 1.42 (t, 3H, J=7.6 Hz), and 1.33 (s, 12H).

### Preparation Example 11

### Preparation of (5-(4-(4,4,5,5-tetramethyl-1,3,2-dioxaborolan-2-yl)phenyl)isoxazol-3-yl)methanol (12i)

### Step 1: Preparation of (5-(4-bromophenyl)isoxazol-3-yl)methanol (12f)

Ethyl 5-(4-bromophenyl)isoxazole-3-carboxylate (2.0 g, 6.75 mmol) synthesized in Step 2 of Preparation Example 10 was dissolved in tetrahydrofuran (40 mL), and 2 equivalents of lithium aluminum hydride (LAH) were gradually added to the solution at 0° C. After reaction for 1 hour, 1N—NaOH and 1N—HCl were added to terminate the reaction, followed by extraction with ethyl acetate (100 mL). The organic layer was washed with water (100 mL×2) and brine (50 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)isoxazol-3-yl)methanol(12f). Yield: 70%.

¹H NMR (CDCl₃, 400 MHz): 7.60 (m, 4H), 6.57 (s, 1H), and 4.79 (s, 2H).

### Step 2: Preparation of 5-(4-(4,4,5,5-tetramethyl-1,3,2-dioxaborolan-2-yl)phenyl)isoxazol-3-yl)methanol (12i)

Analogously to Step 4 of Preparation Example 6, 5-(4-(4,4,5,5-tetramethyl-1,3,2-dioxaborolan-2-yl)phenyl)isoxazol-3-yl)methanol (12i) was prepared from (5-(4-bromophenyl)isoxazol-3-yl)methanol (1.0 g, 3.94 mmol). Yield: 75%.

¹H NMR (CDCl₃, 400 MHz): 7.75 (d, 2H, J=8.4 Hz), 7.61 (d, 2H, J=8.4 Hz), 6.85 (s, 1H), 4.42 (q, 2H, J=6.8 Hz), 1.42 (t, 3H, J=7.6 Hz), and 1.35 (s, 12H).

### Preparation Example 12

### Preparation of 3-(methoxymethyl)-5-(4-(4,4,5,5-tetramethyl-1,3,2-dioxaborolan-2-yl)phenyl)isoxazole (12i)

### Step 1: Preparation of 5-(4-bromophenyl)-3-(methoxymethyl)isoxazole (12g)

5-(4-bromophenyl)isoxazol-3-yl)methanol (1.0 g, 3.94 mmol) synthesized in Step 1 of Preparation Example 11, and 60% sodium hydride (200 mg) were added to N,N-dimethylformamide (50 mL) and the mixture was stirred for 11 min. Methyl iodide was added to the mixture, followed by reaction for 1 hour. After completion of the reaction was confirmed by TLC, extraction was carried with water (20 mL) and ethyl acetate (100 mL). The organic layer was washed with water (50 mL×2) and brine (20 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)-3-(methoxymethyl)isoxazole (12g). Yield: 90%.

¹H NMR (CDCl₃, 400 MHz): 7.63 (m, 4H), 6.67 (s, 1H), 4.55 (s, 2H), and 3.41 (s, 3H).

### Step 2: Preparation of 3-(methoxymethyl)-5-(4-(4,4,5,5-tetramethyl-1,3,2-dioxaborolan-2-yl)phenyl)isoxazole (12i)

Analogously to Step 4 of Preparation Example 6, 3-(methoxymethyl)-5-(4-(4,4,5,5-tetramethyl-1,3,2-dioxaborolan-2-yl)phenyl)isoxazole (12j) was prepared from 5-(4-bromophenyl)-3-(methoxymethyl)isoxazole (700 mg, 2.61 mmol). Yield: 83%.

¹H NMR (CDCl₃, 400 MHz): 7.87 (d, 2H, J=8.4 Hz), 7.76 (d, 2H, J=8.4 Hz), 6.06 (s, 1H), 4.55 (s, 2H), 3.41 (s, 3H), and 1.32 (s, 12H).

### Preparation Example 13

### Preparation of N-methyl-5-(4-(4,4,5,5-tetramethyl-1,3,2-dioxaborolan-2-yl)phenyl)isoxazole-3-carboxamide (12m)

### Step 1: Preparation of 5-(4-bromophenyl)isoxazole-3-carboxylic acid (12k)

Ethyl 5-(4-bromophenyl)isoxazole-3-carboxylate (3.5 g, 11.8 mmol) synthesized in Step 2 of Preparation Example 10 was reacted in a mixture of tetrahydrofuran (20 mL), ethanol (20 mL) and 1N NaOH (20 mL) at 60° C. for 2 hours. After completion of the reaction was confirmed by TLC, the organic solvent was removed under reduced pressure and the reactants were neutralized with addition of 1N—HCl, followed by extraction with water (20 mL) and ethyl acetate (20 mL). The organic layer was washed with water (20 mL) and brine (20 mL). The organic layer was separated, dried over anhydrous magnesium sulfate, and filtered under reduced pressure to remove ethyl acetate. The residue was purified by silica gel column chromatography using methanol and methylene chloride as a developing solvent, thus affording the title compound 5-(4-bromophenyl)isoxazole-3-carboxylic acid (12k). Yield: 56%.

### Step 2: Preparation of 5-(4-bromophenyl)-N-methylisoxazole-3-carboxamide (12l)

5-(4-bromophenyl)isoxazole-3-carboxylic acid (500 mg, 1.87 mmol) and oxalyl dichloride (5 mL) were added to tetrahydrofuran (50 mL), followed by reflux for 1 hour. The solvent was removed under reduced pressure, and tetrahydrofuran (20 mL), triethylamine (Et₃N, 2 mL) and methylene chloride (100 mg) were added dropwise to the reactants. After completion of the reaction was confirmed by TLC, extraction was carried out with water (20 mL) and ethyl acetate (20 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)-N-methylisoxazole-3-carboxamide (121). Yield: 20%.

¹H NMR (CDCl₃, 400 MHz): 7.63 (m, 4H), 6.94 (s, 1H), 6.80 (br, 1H), and 3.02 (d, 3H, J=5.2 Hz).

### Step 3: Preparation of N-methyl-5-(4-(4,4,5,5-tetramethyl-1,3,2-dioxaborolan-2-yl)phenyl)isoxazole-3-carboxamide (12m)

Analogously to Step 4 of Preparation Example 6, N-methyl-5-(4-(4,4,5,5-tetramethyl-1,3,2-dioxaborolan-2-yl)phenyl)isoxazole-3-carboxamide (12m) was prepared from 5-(4-bromophenyl)-N-methylisoxazole-3-carboxamide (105 mg, 0.37 mmol). Yield: 82%.

MS (ESI⁺) m/z 329.1 (M⁺¹)

### Preparation Example 14

### Preparation of 2-methyl-5-(4-(4,4,5,5-tetramethyl-1,3,2-dioxaborolan-2-yl)phenyl)-2H-tetrazole (13b)

### Step 1: Preparation of 5-(4-bromophenyl)-2-methyl-2H-tetrazole (13a)

To N,N-dimethylformamide (10 mL) were added 5-(4-bromophenyl)-1H-tetrazole (5 g, 26.66 mmol), sodium hydroxide (1.6 g, 40.0 mmol) and methyl iodide (5.8 mL, 79.78 mmol) which were then stirred for 4 hours. After completion of the reaction was confirmed by TLC, extraction was carried out with water (20 mL) and ethyl acetate (20 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)-2-methyl-2H-tetrazole (13a). Yield: 40%.

¹H NMR (CDCl₃, 400 MHz): 7.98 (d, 2H, J=8.4 Hz), 7.60 (d, 2H, J=8.4 Hz), and 4.38 (s, 3H).

### Step 2: Preparation of 2-methyl-5-(4-(4,4,5,5-tetramethyl-1,3,2-dioxaborolan-2-yl)phenyl)-2H-tetrazole (13b)

Analogously to Step 4 of Preparation Example 6, 2-methyl-5-(4-(4,4,5,5-tetramethyl-1,3,2-dioxaborolan-2-yl)phenyl)-2H-tetrazole (13b) was prepared from 5-(4-bromophenyl)-2-methyl-2H-tetrazole (1.5 g, 6.27 mmol). Yield: 82%.

¹H NMR (CDCl₃, 400 MHz): 8.12 (d, 2H, J=8.4 Hz), 7.90 (d, 2H, J=8.4 Hz), 4.39 (s, 3H), and 1.32 (s, 12H).

### Preparation Example 15

### Preparation of 1-methyl-5-(4-(4,4,5,5-tetramethyl-1,3,2-dioxaborolan-2-yl)phenyl)-1H-tetrazole (13b-1)

### Step 1: Preparation of 5-(4-bromophenyl)-1-methyl-1H-tetrazole (13a-1)

Analogously to Step 1 of Preparation Example 14, 5-(4-bromophenyl)-1-methyl-1H-tetrazole (13a-1) was prepared using 5-(4-bromophenyl)-1H-tetrazole (5 g, 26.66 mmol) as a starting material.

¹H NMR (CDCl₃, 400 MHz): 7.70 (d, 2H, J=8.8 Hz), 7.61 (d, 2H, J=8.4 Hz), and 4.16 (s, 3H).

### Step 2: Preparation of 1-methyl-5-(4-(4,4,5,5-tetramethyl-1,3,2-dioxaborolan-2-yl)phenyl)-1H-tetrazole (13b-1)

Analogously to Step 4 of Preparation Example 6, 1-methyl-5-(4-(4,4,5,5-tetramethyl-1,3,2-dioxaborolan-2-yl)phenyl)-1H-tetrazole (13b-1) was prepared from 5-(4-bromophenyl)-1-methyl-1H-tetrazole (1.8 g, 7.53 mmol). Yield: 30%.

¹H NMR (CDCl₃, 400 MHz): 7.92 (d, 2H, J=8.8 Hz), 7.81 (d, 2H, J=8.4 Hz), 4.17 (s, 3H), and 1.33 (s, 3H).

### Preparation Example 16

### Preparation of 2-isopropyl-5-(4-(4,4,5,5-tetramethyl-1,3,2-dioxaborolan-2-yl)phenyl)-2H-tetrazole (13d)

### Step 1: Preparation of 5-(4-bromophenyl)-2-isopropyl-2H-tetrazole (13c)

To N,N-dimethylformamide (10 mL) were added 5-(4-bromophenyl)-1H-tetrazole (500 mg, 2.22 mmol), sodium hydroxide (222 mg, 5.55 mmol) and 2-iodopropane (1.13 mL, 6.66 mmol) which were then stirred for 4 hours. After completion of the reaction was confirmed by TLC, reactants were extracted with water (20 mL) and ethyl acetate (20 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)-2-isopropyl-2H-tetrazole (13c). Yield: 68%.

¹H NMR (CDCl₃, 400 MHz): 8.02 (d, 2H, J=9.2 Hz), 7.62 (m, 2H), 5.10 (m, 1H), and 1.72 (d, 6H, J=10.4 Hz).

### Step 2: Preparation of 2-isopropyl-5-(4-(4,4,5,5-tetramethyl-1,3,2-dioxaborolan-2-yl)phenyl)-2H-tetrazole (13d)

Analogously to Step 4 of Preparation Example 6, 2-isopropyl-5-(4-(4,4,5,5-tetramethyl-1,3,2-dioxaborolan-2-yl)phenyl)-2H-tetrazole (13d) was prepared from 5-(4-bromophenyl)-2-isopropyl-2H-tetrazole (407 mg, 1.52 mmol). Yield: 80%.

¹H NMR (CDCl₃, 400 MHz): 8.12 (d, 2H, J=8.4 Hz), 7.82 (d, 2H, J=8.4 Hz), 5.11 (m, 1H), 1.72 (d, 6H, J=10.4 Hz), and 1.35 (s, 12H).

### Preparation Example 17

### Preparation of 2-(methoxymethyl)-5-(4-(4,4,5,5-tetramethyl-1,3,2-dioxaborolan-2-yl)phenyl)-2H-tetrazole (13f)

### Step 1: Preparation of 5-(4-bromophenyl)-2-(methoxymethyl)-2H-tetrazole (13e)

5-(4-bromophenyl)-1H-tetrazole (2 g, 8.89 mmol) was dissolved in N,N-dimethylformamide (10 mL) to which bromomethyl methyl ether (2.8 mL, 22.23 mmol) and sodium hydroxide (890 mg, 22.23 mmol) were then added, followed by stirring at room temperature for 4 hours. After completion of the reaction was confirmed by TLC, water (150 mL) and ethyl acetate (300 mL) were added to the reactants, followed by extraction. The organic layer was washed with brine and distilled under reduced pressure. The residue was purified by silica gel column chromatography using ethyl acetate and hexane as a developing solvent, thus affording the title compound 5-(4-bromophenyl)-2-(methoxymethyl)-2H-tetrazole (13e). Yield: 80%.

¹H NMR (CDCl₃, 400 MHz): 8.05 (d, 2H, J=8.8 Hz), 7.62 (d, 2H, J=8.8 Hz), 5.87 (s, 2H), and 3.50 (s, 3H).

### Step 2: Preparation of 2-(methoxymethyl)-5-(4-(4,4,5,5-tetramethyl-1,3,2-dioxaborolan-2-yl)phenyl)-2H-tetrazole (13f)

Analogously to Step 4 of Preparation Example 6, 2-(methoxymethyl)-5-(4-(4,4,5,5-tetramethyl-1,3,2-dioxaborolan-2-yl)phenyl)-2H-tetrazole (13f) was prepared from 5-(4-bromophenyl)-2-(methoxymethyl)-2H-tetrazole (1.1 g, 4.09 mmol). Yield: 76%.

¹H NMR (CDCl₃, 400 MHz): 8.08 (d, 2H, J=8.8 Hz), 7.77 (d, 2H, J=8.8 Hz), 5.86 (s, 2H), 3.51 (s, 3H), and 1.35 (s, 12H).

### Preparation Example 18

### Preparation of (5-(4-(4,4,5,5-tetramethyl-1,3,2-dioxaborolan-2-yl)phenyl)-2H-tetrazol-2-yl)methanol (13g)

2-(methoxymethyl)-5-(4-(4,4,5,5-tetramethyl-1,3,2-dioxaborolan-2-yl)phenyl)-2H-tetrazole (500 mg, 1.58 mmol) synthesized in Step 2 of Preparation Example 17 was dissolved in methylene chloride (20 mL) to which tribromoborane (BBr₃, 2 eq.) was then added, followed by reaction for 5 hours. After completion of the reaction was confirmed by TLC, water (10 mL) and methylene chloride (30 mL) were added to the reactants, followed by extraction. The organic layer was washed with brine and distilled under reduced pressure. The residue was purified by silica gel column chromatography using ethyl acetate and hexane as a developing solvent, thus affording the title compound (5-(4-(4,4,5,5-tetramethyl-1,3,2-dioxaborolan-2-yl)phenyl)-2H-tetrazol-2-yl)methanol (13g). Yield: 40%.

MS (ESI⁺) m/z 303.1 (M⁺¹)

### Preparation Example 19

### Preparation of N-methyl-N-(4-(4,4,5,5-tetramethyl-1,3,2-dioxaborolan-2-yl)phenyl)acetamide (14a)

N-(4-(4,4,5,5-tetramethyl-1,3,2-dioxaborolan-2-yl)phenyl)acetamide (1.0 g, 3.83 mmol), methyl iodide (1.2 eq.) and triethylamine were dissolved in tetrahydrofuran (10 mL), and the reactants were stirred at room temperature for 4 hours. After completion of the reaction was confirmed by TLC, water (50 mL) and ethyl acetate (500 mL) were added to the reactants, followed by extraction. The organic layer was washed with brine and distilled under reduced pressure. The residue was purified by silica gel column chromatography using ethyl acetate and hexane as a developing solvent, thus affording the title compound N-methyl-N-(4-(4,4,5,5-tetramethyl-1,3,2-dioxaborolan-2-yl)phenyl)acetamide (14a). Yield: 80%.

MS (ESI⁺) m/z 276.1 (M⁺¹)

### Preparation Example 20

### Preparation of 2-methyl-6-(4-(4,4,5,5-tetramethyl-1,3,2-dioxaborolan-2-yl)phenyl)-4,5-dihydropyridazin-3(2H)-one (15b)

Analogously to Step 1 of Preparation Example 19, the title compound 2-methyl-6-(4-(4,4,5,5-tetramethyl-1,3,2-dioxaborolan-2-yl)phenyl)-4,5-dihydropyridazin-3(2H)-one (15b) was prepared from 6-(4-bromophenyl)-4,5-dihydropyridazin-3(2H)-one (1.0 g, 3.96 mmol). Yield: 86%.

MS (ESI⁺) m/z 315.1 (M⁺¹)

### Preparation Example 21

### Preparation of 5-methyl-3-(4-(4,4,5,5-tetramethyl-1,3,2-dioxaborolan-2-yl)phenyl)-1,2,4-oxadiazole (16c)

### Step 1: Preparation of 4-bromo-N′-hydroxybenzimidamide (16a)

4-bromobenzonitrile (3 g, 16.48 mmol), hydroxylamine hydrochloride (1.49 g, 21.4 mmol) and NaHCO₃ (2.08 g, 25 mmol) were dissolved in ethanol (70 mL), and the solution was warmed to 90° C., followed by reaction for 3 hours. After completion of the reaction was confirmed by TLC, extraction was carried out with water (100 mL) and ethyl acetate (250 mL). The organic layer was washed with brine and distilled under reduced pressure. The resulting solids were washed with hexane, filtered and dried under vacuum to afford the title compound 4-bromo-N′-hydroxybenzimidamide (16a). Yield: 90%.

MS (ESI⁺) m/z 215.1 (M⁺¹)

### Step 2: Preparation of 3-(4-bromophenyl)-5-methyl-1,2,4-oxadiazole (16b)

4-bromo-N′-hydroxybenzimidamide (1.56 g, 7.25 mmol) and N,N-dimethylacetamide dimethyl acetal (DMA acetal, 2.9 mL, 21.8 mmol) were dissolved in dioxane (30 μL), followed by reaction under reflux for 12 hours. After completion of the reaction was confirmed by TLC, water (50 mL) and ethyl acetate (100 mL) were added to the reactants, followed by extraction. The organic layer was washed with brine and distilled under reduced pressure. The resulting solids were dried and recrystallized from hexane to afford the title compound 3-(4-bromophenyl)-5-methyl-1,2,4-oxadiazole (16b). Yield: 85%.

MS (ESI⁺) m/z 239.1 (M⁺¹)

### Step 3: Preparation of 5-methyl-3-(4-(4,4,5,5-tetramethyl-1,3,2-dioxaborolan-2-yl)phenyl)-1,2,4-oxadiazole (16c)

Analogously to Step 4 of Preparation Example 6, 5-methyl-3-(4-(4,4,5,5-tetramethyl-1,3,2-dioxaborolan-2-yl)phenyl)-1,2,4-oxadiazole (16c) was prepared from 3-(4-bromophenyl)-5-methyl-1,2,4-oxadiazole (1.18 g, 6.61 mmol). Yield: 80%.

¹H NMR (CDCl₃, 400 MHz): 8.03 (d, 2H, J=8.0 Hz), 7.89 (d, 2H, J=8.8 Hz), 2.63 (s, 3H), 1.34, and 1.24 (each s, 12H).

### Preparation Example 22

### Preparation of 2-methyl-5-(4-(4,4,5,5-tetramethyl-1,3,2-dioxaborolan-2-yl)phenyl)-1,3,4-oxadiazole (17b)

### Step 1: Preparation of 2-(4-bromophenyl)-5-methyl-1,3,4-oxadiazole (17a)

5-(4-bromophenyl)-2H-tetrazole (2.0 g, 8.89 mmol) was added to AcO₂ in pyridine, followed by reaction under reflux for 2 hours. After completion of the reaction was confirmed by TLC, water (50 mL) and ethyl acetate (500 mL) were added to the reactants, followed by extraction. The organic layer was washed with brine and distilled under reduced pressure. The residue was purified by silica gel column chromatography using ethyl acetate and hexane as a developing solvent, thus affording the title compound 2-(4-bromophenyl)-5-methyl-1,3,4-oxadiazole (17a). Yield: 60%.

¹H NMR (CDCl₃, 400 MHz): 7.88 (d, 2H, J=8.8 Hz), 7.62 (d, 2H, J=8.8 Hz), and 2.60 (s, 3H).

### Step 2: Preparation of 2-methyl-5-(4-(4,4,5,5-tetramethyl-1,3,2-dioxaborolan-2-yl)phenyl)-1,34-oxadiazole (17b)

Analogously to Step 4 of Preparation Example 6, the title compound 2-methyl-5-(4-(4,4,5,5-tetramethyl-1,3,2-dioxaborolan-2-yl)phenyl)-1,3,4-oxadiazole (17b) was prepared from 2-(4-bromophenyl)-5-methyl-1,3,4-oxadiazole (1.0 g, 4.18 mmol). Yield: 70%.

MS (ESI⁺) m/z 287.1 (M⁺¹)

### Preparation Example 23

### Preparation of 2-(4-(4,4,5,5-tetramethyl-1,3,2-dioxaborolan-2-VI)phenyl)-5-(trifluoromethyl)-1,3,4-oxadiazole (18b)

### Step 1: Preparation of 2-(4-bromophenyl)-5-(trifluoromethyl)-1,3,4-oxadiazole (18a)

5-(4-bromophenyl)-1H-tetrazole (3 g, 13.3 mmol) and (CF₃CO)₂O (9 mL, 40 mmol) were dissolved in pyridine (10 mL), followed by reflux for 12 hours. After completion of the reaction was confirmed by TLC, extraction was carried out with water (20 mL) and ethyl acetate (50 mL). The organic layer was washed with water (20 mL×2) and brine (20 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 2-(4-bromophenyl)-5-(trifluoromethyl)-1,3,4-oxadiazole (18a). Yield: 58%.

MS (ESI⁺) m/z 293.1 (M⁺¹)

### Step 2: Preparation of 2-(4-(4,4,5,5-tetramethyl-1,3,2-dioxaborolan-2-yl)phenyl)-5-(trifluoromethyl)-1,3,4-oxadiazole (18b)

Analogously to Step 4 of Preparation Example 6, the title compound 2-(4-(4,4,5,5-tetramethyl-1,3,2-dioxaborolan-2-yl)phenyl)-5-(trifluoromethyl)-1,3,4-oxadiazole (18b) was prepared from 2-(4-bromophenyl)-5-(trifluoromethyl)-1,3,4-oxadiazole (2.22 g, 7.58 mmol). Yield: 85%.

¹H NMR (CDCl₃, 400 MHz): 8.09 (d, 2H, J=6.4 Hz), 7.96 (d, 2H, J=6.8 Hz), and 1.33 (s, 12H).

### Preparation Example 24

### Preparation of 2-(4-(4,4,5,5-tetramethyl-1,3,2-dioxaborolan-2-yl)phenyl)-1,3,4-oxadiazole (19d)

### Step 1: Preparation of ethyl 4-bromobenzoate (19a)

4-bromobenzoic acid (5 g, 25.9 mmol) and H₂SO₄ (10 mL) were added to ethanol (100 mL), and the solution was warmed to 100° C., followed by reaction for 6 hours. After completion of the reaction was confirmed by TLC, extraction was carried out with water (10 mL) and ethyl acetate (300 mL). The organic layer was washed with brine and distilled under reduced pressure. The resulting solids were washed with hexane, filtered and dried under vacuum to afford the title compound ethyl 4-bromobenzoate (19a). Yield: 60%.

### Step 2: Preparation of 4-bromobenzohydrazide (19b)

4-bromobenzoate (1.5 g, 6.52 mmol) and NH₂NH₂ (3.5 mL) were dissolved in ethanol (50 mL), followed by reaction under reflux for 12 hours. After completion of the reaction was confirmed by TLC, extraction was carried out with water (100 mL) and ethyl acetate (200 mL). The organic layer was washed with brine and distilled under reduced pressure. The resulting solids were washed with hexane, filtered and dried under vacuum to afford the title compound 4-bromobenzohydrazide (19b). Yield: 82%.

¹H NMR (CDCl₃, 400 MHz): 9.75 (s, 1H), 7.87 (d, 2H, J=8.0 Hz), 7.69 (d, 2H, J=8.0 Hz), and 4.26 (bs, 2H).

### Step 3: Preparation of 2-(4-bromophenyl)-1,3,4-oxadiazole (19c)

4-bromobenzohydrazide (1.23 g, 5.7 mmol) and acetic anhydride (AC₂O, 1 mL) were dissolved in dioxane (10 mL), followed by reaction under reflux for 4 hours. After completion of the reaction was confirmed by TLC, extraction was carried out with water (100 mL) and ethyl acetate (150 mL). The organic layer was washed with brine and distilled under reduced pressure. The resulting solids were washed with hexane, filtered and dried under vacuum to afford the title compound 2-(4-bromophenyl)-1,3,4-oxadiazole (19c). Yield: 60%.

¹H NMR (CDCl₃, 400 MHz): 8.55 (s, 1H), 7.88 (d, 2H, J=8.0 Hz), and 7.61 (d, 2H, J=8.0 Hz).

### Step 4: Preparation of 2-(4-(4,4,5,5-tetramethyl-1,3,2-dioxaborolan-2-yl)phenyl)-1,3,4-oxadiazole (19d)

Analogously to Step 4 of Preparation Example 6, the title compound 2-(4-(4,4,5,5-tetramethyl-1,3,2-dioxaborolan-2-yl)phenyl)-1,3,4-oxadiazole (19d) was prepared from 2-(4-bromophenyl)-1,3,4-oxadiazole (500 mg, 2.22 mmol). Yield: 85%.

MS (ESI⁺) m/z 273.1 (M⁺¹)

### Preparation Example 25

### Preparation of 4,5-dimethyl-2-(4-(4,4,5,5-tetramethyl-1,3,2-dioxaborolan-2-yl)phenyl)oxazole (20e)

### Step 1: Preparation of methyl 2-(4-bromobenzamido)propanoate (20a)

4-bromobenzoic acid (5 g, 25.9 mmol), triethylamine (1.2 eq.), alanine methyl ester (1.1 eq.), and ethyl chloroformate (1.1 eq.) were stirred in a mixed solvent of tetrahydrofuran (20 mL) and methanol (10 mL) at room temperature for 4 hours. After completion of the reaction was confirmed by TLC, water (150 mL) and ethyl acetate (300 mL) were added to the reactants, followed by extraction. The organic layer was washed with brine and distilled under reduced pressure. The residue was purified by silica gel column chromatography using ethyl acetate and hexane as a developing solvent, thus affording the title compound methyl 2-(4-bromobenzamido)propanoate (20a). Yield: 60%.

¹H NMR (DMSO-d₆, 400 MHz): 8.77 (d, 1H, J=6.0 Hz), 7.83 (d, 2H, J=8.0 Hz), 7.68 (d, 2H, J=8.0 Hz), 4.36 (m, 1H), 3.67 (s, 3H), and 1.37 (m, 3H).

### Step 2: Preparation of 2-(4-bromobenzamido)propionic acid (20b)

2-(4-bromobenzamido)propanoate (3 g, 6.99 mmol) was added to methanol (5 mL) to which 1N NaOH (2 mL) was then added, followed by reaction under reflux for 2 hours. After completion of the reaction was confirmed by TLC, the organic solvent was removed under reduced pressure and the residue was neutralized with addition of 1N—HCl, followed by extraction with water (10 mL) and ethyl acetate (20 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using methanol and methylene chloride as a developing solvent, thus affording the title compound 2-(4-bromobenzamido)propionic acid (20b). Yield: 90%.

¹H NMR (DMSO-d₆, 400 MHz): 12.57 (s, 1H), 8.79 (d, 1H, J=6.0 Hz), 7.83 (d, 2H, J=8.0 Hz), 7.68 (d, 2H, J=8.0 Hz), 4.38 (m, 1H), and 1.37 (m, 3H).

### Step 3: Preparation of 4-bromo-N-(3-oxobutan-2-yl)benzamide (20c)

2-(4-bromobenzamido)propionic acid (1.0 g, 3.68 mmol) and acetic anhydride (AC₂O, 1 mL) were reacted under reflux in pyridine (10 mL) for 3 hours. After completion of the reaction was confirmed by TLC, extraction was carried out with water (100 mL) and ethyl acetate (150 mL). The organic layer was washed with brine and distilled under reduced pressure. The resulting solids were washed with hexane, filtered and dried under vacuum to afford the title compound 4-bromo-N-(3-oxobutan-2-yl)benzamide (20c). Yield: 60%.

¹H NMR (DMSO-d₆, 400 MHz): 8.82 (d, 1H, J=6.0 Hz), 7.82 (d, 2H, J=8.0 Hz), 7.67 (d, 2H, J=8.0 Hz), 4.40 (m, 1H), 2.10 (s, 1H), and 1.28 (m, 3H).

### Step 4: Preparation of 2-(4-bromophenyl)-4,5-dimethyloxazole (20d)

4-bromo-N-(3-oxobutan-2-yl)benzamide (1.0 g, 3.97 mmol) was reacted in a mixture of acetic anhydride (AC₂O, 2 mL) and sulfuric acid at 90° C. for 1.5 hours. Water (30 mL) was added to the reactants to result in solidification. The resulting solids were filtered, dissolved in ethyl acetate, and purified by silica gel column chromatography using ethyl acetate and hexane as a developing solvent, thus affording the title compound 2-(4-bromophenyl)-4,5-dimethyloxazole (20d). Yield: 70%.

¹H NMR (DMSO-d₆, 400 MHz): 7.81 (d, 2H, J=8.0 Hz), 7.67 (d, 2H, J=8.0 Hz), 2.30 (s, 3H), and 2.08 (s, 3H).

### Step 5: Preparation of 4,5-dimethyl-2-(4-(4,4,5,5-tetramethyl-1,3,2-dioxaborolan-2-yl)phenyl)oxazole (20e)

Analogously to Step 4 of Preparation Example 6, the title compound 4,5-dimethyl-2-(4-(4,4,5,5-tetramethyl-1,3,2-dioxaborolan-2-yl)phenyl)oxazole (20e) was prepared from 2-(4-bromophenyl)-4,5-dimethyloxazole (500 mg, 1.98 mmol). Yield: 85%.

¹H NMR (CDCl₃, 400 MHz): 7.95 (d, 2H, J=8.0 Hz), 7.83 (d, 2H, J=8.0 Hz), 2.30 (s, 3H), 2.14 (s, 3H), and 1.34 (s, 12H).

### Preparation Example 26

### Preparation of 1,3-dimethyl-5-(4-(4,4,5,5-tetramethyl-1,3,2-dioxaborolan-2-yl)phenyl)-1H-pyrazole (21c)

### Step 1: Preparation of 5-(4-bromophenyl)-3-methyl-1H-pyrazole (21a)

1-(4-bromophenyl)-3-(dimethylamino)but-2-en-1-one (2.68 g, 10 mmol) synthesized in Step 1 of Preparation Example 9 was dissolved in ethanol (10 mL) to which hydrazine (2 eq.) was then added, followed by reaction at 90° C. for 6 hours. After completion of the reaction was confirmed by TLC, reactants were filtered through celite. The filtrate was extracted with water (100 mL) and ethyl acetate (100 mL). The organic layer was washed with water (100 mL×2) and brine (50 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)-3-methyl-1H-pyrazole (21a). Yield: 70%.

MS (ESI⁺) m/z 237.1 (M⁺¹)

### Step 2: Preparation of 5-(4-bromophenyl)-1,3-dimethyl-1H-pyrazole (21b)

5-(4-bromophenyl)-3-methyl-1H-pyrazole (1.0 g, 4.22 mmol) was dissolved in N,N-dimethylformamide (10 mL) to which 60% sodium hydride (NaH, 1.3 eq.) was then added, followed by addition of methyl iodide (1.5 eq.) and reaction at room temperature for 1 hour. After completion of the reaction was confirmed by TLC, the reactants were filtered through celite. The filtrate was extracted with water (50 mL) and ethyl acetate (100 mL). The organic layer was washed with water (100 mL×2) and brine (50 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound 5-(4-bromophenyl)-1,3-dimethyl-1H-pyrazole (21b). Yield: 70%.

¹H NMR (CDCl₃, 400 MHz): 7.61 (d, 2H, J=8.0 Hz), 7.45 (d, 2H, J=8.0 Hz), 3.79 (s, 3H), and 2.29 (s, 3H).

### Step 3: Preparation of 1,3-dimethyl-5-(4-(4,4,5,5-tetramethyl-1,3,2-dioxaborolan-2-yl)phenyl)-1H-pyrazole (21c)

Analogously to Step 4 of Preparation Example 6, the title compound 1,3-dimethyl-5-(4-(4,4,5,5-tetramethyl-1,3,2-dioxaborolan-2-yl)phenyl)-1H-pyrazole (21c) was prepared from 5-(4-bromophenyl)-1,3-dimethyl-1H-pyrazole (900 mg, 3.58 mmol). Yield: 85%.

MS (ESI⁺) m/z 299.1 (M⁺¹)

### Example 1

### Preparation of (S)-2-ethoxy-3-(4-((5-(3-methoxyphenyl)-3-methylthiophen-2-yl)methoxy)phenyl)propionic acid

### Step 1: Preparation of (S)-2-ethoxy-3-(4-((5-(3-methoxyphenyl)-3-methylthiophen-2-yl)methoxy)phenyl)propionic acid ethyl ester

(S)-3-(4-((5-bromo-3-methylthiophen-2-yl)methoxy)phenyl)-2-ethoxypropionic acid ethyl ester (7b, 500 mg, 1.17 mmol) synthesized in Step 3 of Preparation Example 2, 3-methoxyphenylboronic acid (230 mg, 1.52 mmol), and cesium carbonate (2 eq.), and tetrakis(triphenylphosphine)palladium (160 mg, 0.14 mmol) were reacted in dioxane (20 mL) at 90° C. for 2 hours, and the reactants were then cooled to room temperature. Water was added to the reactants, followed by extraction with ethyl acetate (50 mL). The organic layer was washed with water (30 mL×2) and brine (30 mL). The organic layer was separated, dried over anhydrous magnesium sulfate, and filtered under reduced pressure to remove ethyl acetate. The residue was purified by silica gel column chromatography using ethyl acetate and hexane as a developing solvent, thus affording the title compound (S)-2-ethoxy-3-(4-((5-(3-methoxyphenyl)-3-methylthiophen-2-yl)methoxy)phenyl)propionic acid ethyl ester. Yield: 60%.

¹H NMR (CDCl₃, 400 MHz): 7.26 (m, 1H), 7.16 (m, 3H), 7.09 (m, 1H), 6.93 (d, 2H, J=4.4 Hz), 6.92 (m, 1H), 4.15 (q, 2H, J=6.8 Hz), 4.06 (q, 1H, J=4.4 Hz), 3.83 (s, 3H), 3.60 (m, 1H), 3.46 (m, 1H), 3.07 (m, 1H), 2.99 (m, 1H), 2.29 (s, 3H), 1.23 (t, 3H, J=7.2 Hz), and 1.16 (t, 3H, J=5.2 Hz).

### Step 2: Preparation of (S)-2-ethoxy-3-(4-((5-(3-methoxyphenyl)-3-methylthiophen-2-yl)methoxy)phenyl)propionic acid

(S)-2-ethoxy-3-(4-((5-(3-methoxyphenyl)-3-methylthiophen-2-yl)methoxy)phenyl)propionic acid ethyl ester (300 mg, 0.66 mmol) prepared in Step 1 of Example 1 was reacted with tetrahydrofuran (3 mL), ethanol (1 mL) and 1N NaOH (2 mL) at 60° C. for 2 hours. After completion of the reaction was confirmed by TLC, the organic solvent was removed under reduced pressure and the reactants were neutralized with addition of 1N—HCl, followed by extraction with water (10 mL) and ethyl acetate (20 mL). The organic layer was washed with water (10 mL×2) and brine (10 mL). The organic layer was separated, dried over anhydrous magnesium sulfate, and filtered under reduced pressure to remove ethyl acetate. The residue was purified by silica gel column chromatography using methanol and methylene chloride as a developing solvent, thus affording the title compound (S)-2-ethoxy-3-(4-((5-(3-methoxyphenyl)-3-methylthiophen-2-yl)methoxy)phenyl)propionic acid (Example 1). Yield: 90%.

¹H NMR (CDCl₃, 400 MHz): 7.26 (m, 1H), 7.16 (m, 3H), 7.09 (m, 1H), 6.93 (d, 2H, J=4.4 Hz), 6.92 (m, 1H), 4.06 (q, 1H, J=4.4 Hz), 3.83 (s, 3H), 3.60 (m, 1H), 3.46 (m, 1H), 3.07 (m, 1H), 2.99 (m, 1H), 2.29 (s, 3H), and 1.16 (t, 3H, J=5.2 Hz).

### Example 2

### Preparation of (S)-2-ethoxy-3-(4-((5-(4-fluorophenyl)-3-methylthiophen-2-yl)methoxy)phenyl)propionic acid

Analogously to Step 1 of Example 1, an ester compound was prepared from (S)-2-ethoxy-3-(4-((5-(4-fluorophenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (600 mg) and 4-fluorophenylboronic acid (1.2 eq.). Analogously to Step 2 of Example 1, the ester compound was then hydrolyzed to afford the title compound (S)-2-ethoxy-3-(4-((5-(4-fluorophenyl)-3-methylthiophen-2-yl)methoxy)phenyl)propionic acid (Example 2).

¹H NMR (CDCl₃, 400 MHz): 7.51 (m, 1H), 7.17 (d, 2H, J=7.6 Hz), 7.05 (m, 3H), 6.92 (d, 2H, J=6.4 Hz), 5.11 (s, 2H), 4.06 (q, 1H, J=4.4 Hz), 3.58 (m, 1H), 3.48 (m, 1H), 3.07 (m, 1H), 2.98 (m, 1H), 2.25 (s, 3H), and 1.18 (t, 3H, J=4.0 Hz).

### Example 3

### Preparation of (S)-3-(4-((5-(3,4-dimethoxyphenyl)-3-methylthiophen-2-yl)methoxy)phenyl)-2-ethoxypropionic acid

Analogously to Step 1 of Example 1, an ester compound (S)-3-(4-((5-(3,4-dimethoxyphenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (300 mg) and 3,4-dimethoxyphenylboronic acid (1.2 eq.). Analogously to Step 2 of Example 1, the ester compound was then hydrolyzed to afford the title compound (S)-3-(4-((5-(3,4-dimethoxyphenyl)-3-methylthiophen-2-yl)methoxy)phenyl)-2-ethoxypropionic acid (Example 3).

¹H NMR (CDCl₃, 400 MHz): 7.17 (m, 8H), 5.06 (s, 2H), 4.05 (q, 1H, J=4.4 Hz), 3.90 (s, 3H), 3.88 (s, 3H), 3.58 (m, 1H), 3.47 (m, 1H), 3.06 (m, 1H), 2.97 (m, 1H), 2.25 (s, 3H), and 1.17 (t, 3H, J=5.2 Hz).

### Example 4

### Preparation of (S)-3-(4-((5-(4-methoxyphenyl)-3-methylthiophen-2-yl)methoxy)phenyl)-2-ethoxypropionic acid

Analogously to Step 1 of Example 1, an ester compound (S)-3-(4-((5-(4-methoxyphenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (300 mg) and 3,4-dimethoxyphenylboronic acid (1.2 eq.). Analogously to Step 2 of Example 1, the ester compound was then hydrolyzed to afford the title compound (S)-3-(4-((5-(4-methoxyphenyl)-3-methylthiophen-2-yl)methoxy)phenyl)-2-ethoxypropionic acid (Example 4).

MS (ESI⁺) m/z 427.1 (M⁺¹)

### Example 5

### Preparation of (S)-2-ethoxy-3-(4-((5-(4-ethylphenyl)-3-methylthiophen-2-yl)methoxy)phenyl)propionic acid

Analogously to Step 1 of Example 1, an ester compound (S)-3-(4-((5-(4-methoxyphenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (500 mg) and 4-ethylphenylboronic acid (1.2 eq.). Analogously to Step 2 of Example 1, the ester compound was then hydrolyzed to afford the title compound (S)-2-ethoxy-3-(4-((5-(4-ethylphenyl)-3-methylthiophen-2-yl)methoxy)phenyl)propionic acid (Example 5).

¹H NMR (CDCl₃, 400 MHz): 7.46 (d, 1H, J=6.4 Hz), 7.19 (m, 4H), 7.00 (s, 1H), 6.92 (d, 2H, J=6.4 Hz), 5.06 (s, 2H), 4.05 (q, 1H, J=4.4 Hz), 3.58 (m, 1H), 3.47 (m, 1H), 3.06 (m, 1H), 2.97 (m, 1H), 2.66 (q, 2H, J=7.6 Hz), 2.25 (s, 3H), 1.26 (t, 3H, J=8.4 Hz), and 1.18 (t, 3H, J=6.8 Hz).

### Example 6

### Preparation of (S)-2-ethoxy-3-(4-((3-methyl-5-(4-(trifluoromethyl)phenyl)thiophen-2-VI)methoxy)phenyl)propionic acid

Analogously to Step 1 of Example 1, an ester compound (S)-2-ethoxy-3-(4-((3-methyl-5-(4-(trifluoromethyl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (400 mg) and 4-trifluoromethylphenylboronic acid (1.2 eq.). Analogously to Step 2 of Example 1, the ester compound was then hydrolyzed to afford the title compound (S)-2-ethoxy-3-(4-((3-methyl-5-(4-(trifluoromethyl)phenyl)thiophen-2-yl)methoxy)phenyl)propionic acid (Example 6).

MS (ESI⁺) m/z 465.1 (M⁺¹), 487.1 (M+Na)

### Example 7

### Preparation of (S)-2-ethoxy-3-(4-((3-methyl-5-p-phenylthiophen-2-yl)methoxy)phenyl)propionic acid

Analogously to Step 1 of Example 1, an ester compound (S)-2-ethoxy-3-(4-((3-methyl-5-p-phenylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (400 mg) and 4-methylphenylboronic acid (1.2 eq.). Analogously to Step 2 of Example 1, the ester compound was then hydrolyzed to afford the title compound (S)-2-ethoxy-3-(4-((3-methyl-5-p-phenylthiophen-2-yl)methoxy)phenyl)propionic acid (Example 7).

MS (ESI⁺) m/z 411.1 (M⁺¹).

### Example 8

### Preparation of (S)-2-ethoxy-3-(4-((3-methyl-5-(4-(trifluoromethoxy)phenyl)thiophen-2-yl)methoxy)phenyl)propionic acid

Analogously to Step 1 of Example 1, an ester compound (S)-2-ethoxy-3-(4-((3-methyl-5-(4-(trifluoromethoxy)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (600 mg) and 4-(trifluoromethoxy)phenylboronic acid (1.2 eq.). Analogously to Step 2 of Example 1, the ester compound was then hydrolyzed to afford the title compound (S)-2-ethoxy-3-(4-((3-methyl-5-(4-(trifluoromethoxy)phenyl)thiophen-2-yl)methoxy)phenyl)propionic acid (Example 8).

¹H NMR (CDCl₃, 400 MHz): 7.55 (d, 1H, J=6.4 Hz), 7.19 (m, 4H), 7.02 (s, 1H), 6.92 (d, 2H, J=4.4 Hz), 5.07 (s, 2H), 4.06 (q, 1H, J=4.0 Hz), 3.58 (m, 1H), 3.47 (m, 1H), 3.06 (m, 1H), 2.98 (m, 11H), 2.25 (s, 3H), and 1.18 (t, 3H, J=6.8 Hz).

### Example 9

### Preparation of (S)-2-ethoxy-3-(4-((5-(4-isopropylphenyl)-3-methylthiophen-2-yl)methoxy)phenyl)propionic acid

Analogously to Step 1 of Example 1, an ester compound (S)-2-ethoxy-3-(4-((3-methyl-5-(4-isopropylphenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (600 mg) and 4-isopropylphenylboronic acid (1.2 eq.). Analogously to Step 2 of Example 1, the ester compound was then hydrolyzed to afford the title compound (S)-2-ethoxy-3-(4-((5-(4-isopropylphenyl)-3-methylthiophen-2-yl)methoxy)phenyl)propionic acid (Example 9).

MS (ESI⁺) m/z 439.1 (M⁺¹).

### Example 10

### Preparation of (S)-2-ethoxy-3-(4-((3-methyl-5-phenylthiophen-2-yl)methoxy)phenyl)propionic acid

Analogously to Step 1 of Example 1, an ester compound (S)-2-ethoxy-3-(4-((3-methyl-5-phenylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (400 mg) and phenylboronic acid (1.2 eq.). Analogously to Step 2 of Example 1, the ester compound was then hydrolyzed to afford the title compound (S)-2-ethoxy-3-(4-((3-methyl-5-phenylthiophen-2-yl)methoxy)phenyl)propionic acid (Example 10).

MS (ESI⁺) m/z 397.1 (M⁺¹).

### Example 11

### Preparation of (S)-3-(4-((5-(4-cyanophenyl)-3-methylthiophen-2-yl)methoxy)phenyl)-2-ethoxypropionic acid

Analogously to Step 1 of Example 1, an ester compound (S)-3-(4-((5-(4-cyanophenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (440 mg) and 4-cyanophenylboronic acid (1.2 eq.). Analogously to Step 2 of Example 1, the ester compound was then hydrolyzed to afford the title compound (S)-3-(4-((5-(4-cyanophenyl)-3-methylthiophen-2-yl)methoxy)phenyl)-2-ethoxypropionic acid (Example 11).

MS (ESI⁺) m/z 422.1 (M⁺¹).

### Example 12

### Preparation of (S)-3-(4-((5-(4-acetylphenyl)-3-methylthiophen-2-yl)methoxy)phenyl)-2-ethoxypropionic acid

Analogously to Step 1 of Example 1, an ester compound (S)-3-(4-((5-(4-acetylphenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (440 mg) and 4-acetylphenylboronic acid (1.2 eq.). Analogously to Step 2 of Example 1, the ester compound was then hydrolyzed to afford the title compound (S)-3-(4-((5-(4-acetylphenyl)-3-methylthiophen-2-yl)methoxy)phenyl)-2-ethoxypropionic acid (Example 12).

¹H NMR (CDCl₃, 400 MHz): 7.93 (d, 2H, J=6.4 Hz), 7.63 (d, 2H, J=6.4 Hz), 7.17 (d, 2H, J=8.8 Hz) 6.93 (d, 2H, J=5.2 Hz), 5.09 (s, 2H), 4.07 (q, 1H, J=4.0 Hz), 3.60 (m, 1H), 3.49 (m, 1H), 2.59 (s, 3H), 2.27 (s, 3H), and 1.20 (t, 3H, J=6.4 Hz).

### Example 13

### Preparation of (S)-3-(4-((5-(4-acetamidophenyl)-3-methylthiophen-2-yl)methoxy)phenyl)-2-ethoxypropionic acid

Analogously to Step 1 of Example 1, an ester compound (S)-2-ethoxy-3-(4-((3-methyl-5-(4-(acetamido)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (520 mg) and N-(4-(4,4,5,5-tetramethyl-1,3,2-dioxaborolan-2-yl)phenyl)acetamide (1.2 eq.) as defined in formula 11. Analogously to Step 2 of Example 1, the ester compound was then hydrolyzed to afford the title compound (S)-3-(4-((5-(4-acetamidophenyl)-3-methylthiophen-2-yl)methoxy)phenyl)-2-ethoxypropionic acid (Example 13).

¹H NMR (CDCl₃, 400 MHz): 7.49 (br, 3H), 7.23 (m, 2H), 7.16 (d, 2H, J=8.8 Hz), 6.99 (s, 1H), 6.91 (d, 2H, J=6.8 Hz), 5.10 (s, 2H), 4.06 (q, 1H, J=4.0 Hz), 3.58 (m, 1H), 3.48 (m, 1H), 3.06 (m, 1H), 2.97 (m, 1H), 2.24 (s, 3H), 2.15 (s, 3H), and 1.18 (t, 3H, J=6.8 Hz).

### Example 14

### Preparation of (S)-2-ethoxy-3-(4-((3-methyl-5-(4-(N-methylacetamido)phenyl)thiophen-2-yl)methoxy)phenyl)propionic acid

Analogously to Step 1 of Example 1, an ester compound (S)-3-(4-((5-(4-acetamidophenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (740 mg) and Compound 14a (1.3 eq.) synthesized in Preparation Example 19. Analogously to Step 2 of Example 1, the ester compound was then hydrolyzed to afford the title compound (S)-2-ethoxy-3-(4-((3-methyl-5-(4-(N-methylacetamido)phenyl)thiophen-2-yl)methoxy)phenyl)propionic acid (Example 14).

¹H NMR (CDCl₃, 400 MHz): 7.49 (br, 3H), 7.23 (m, 2H), 7.16 (d, 2H, J=8.8 Hz), 6.99 (s, 1H), 6.91 (d, 2H, J=6.8 Hz), 5.10 (s, 2H), 4.06 (q, 1H, J=4.0 Hz), 3.58 (m, 1H), 3.48 (m, 1H), 3.06 (m, 1H), 2.97 (m, 1H), 2.24 (s, 3H), 2.15 (s, 3H), and 1.18 (t, 3H, J=6.8 Hz).

### Example 15

### Preparation of (S)-3-(4-((5-(4-benzoylphenyl)-3-methylthiophen-2-yl)methoxy)phenyl)-2-ethoxypropionic acid

Analogously to Step 1 of Example 1, an ester compound (S)-3-(4-((5-(4-benzoylphenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (740 mg) and 4-benzoylphenylboronic acid (1.2 eq.). Analogously to Step 2 of Example 1, the ester compound was then hydrolyzed to afford the title compound (S)-3-(4-((5-(4-benzoylphenyl)-3-methylthiophen-2-yl)methoxy)phenyl)-2-ethoxypropionic acid (Example 15).

MS (ESI⁺) m/z 501.1 (M⁺¹).

### Example 16

### Preparation of (S)-2-ethoxy-3-(4-((5-(4-(furan-2-yl-methylcarbamoyl)phenyl)-3-methylthiophen-2-yl)methoxy)phenyl)propionic acid

Analogously to Step 1 of Example 1, an ester compound (S)-2-ethoxy-3-(4-((5-(4-(furan-2-yl-methylcarbamoyl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (400 mg) and 4-(furan-2-yl-methylcarbamoyl)phenylboronic acid (1.2 eq.). Analogously to Step 2 of Example 1, the ester compound was then hydrolyzed to afford the title compound (S)-2-ethoxy-3-(4-((5-(4-(furan-2-yl-methylcarbamoyl)phenyl)-3-methylthiophen-2-yl)methoxy)phenyl)propionic acid (Example 16).

MS (ESI⁺) m/z 520.2 (M⁺¹).

### Example 17

### Preparation of (S)-2-ethoxy-3-(4-((3-methyl-5-(4-(morpholino-4-carbonyl)phenyl)thiophen-2-yl)methoxy)phenyl)propionic acid

Analogously to Step 1 of Example 1, an ester compound (S)-2-ethoxy-3-(4-((3-methyl-5-(4-(morpholine-4-carbonyl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (400 mg) and 4-(morpholino-4-carbonyl)phenylboronic acid (1.2 eq.). Analogously to Step 2 of Example 1, the ester compound was then hydrolyzed to afford the title compound (S)-2-ethoxy-3-(4-((3-methyl-5-(4-(morpholino-4-carbonyl)phenyl)thiophen-2-yl)methoxy)phenyl)propionic acid (Example 17).

¹H NMR (DMSO-d₆, 400 MHz): 7.66 (d, 2H, J=8.0 Hz), 7.43 (d, 2H, J=7.6 Hz), 7.37 (s, 1H), 7.15 (d, 2H, J=8.4 Hz), 6.93 (d, 2H, J=8.4 Hz), 5.16 (s, 2H), 3.76 (br, 24H), 2.80 (m, 2H), 2.23 (s, 3H), 1.81 (br, 18H), and 1.04 (t, 3H, J=6.8 Hz).

### Example 18

### Preparation of (S)-2-ethoxy-3-(4-((3-methyl-5-(4-(morpholinosulfonyl)phenyl)thiophen-2-yl)methoxy)phenyl)propionic acid

Analogously to Step 1 of Example 1, an ester compound (S)-2-ethoxy-3-(4-((3-methyl-5-(4-(morpholinosulfonyl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (330 mg) and 4-(morpholinosulfonyl)phenylboronic acid (1.2 eq.). Analogously to Step 2 of Example 1, the ester compound was then hydrolyzed to afford the title compound (S)-2-ethoxy-3-(4-((3-methyl-5-(4-(morpholinosulfonyl)phenyl)thiophen-2-yl)methoxy)phenyl)propionic acid (Example 18).

¹H NMR (CDCl₃, 400 MHz): 7.72 (m, 4H), 7.18 (m, 3H), 6.93 (d, 2H, J=5.2 Hz), 5.10 (s, 2H), 4.06 (m, 1H), 3.74 (m, 4H), 3.58 (m, 1H), 3.49 (m, 2H), 3.06 (m, 8H), 2.31 (s, 3H), and 1.26 (t, 3H, J=6.0 Hz).

### Example 19

### Preparation of (S)-3-(4-((5-(4-(5,6-dihydro-4H-1,3-oxazin-2-yl)phenyl)-3-methylthiophen-2-yl)methoxy)phenyl)-2-ethoxypropionic acid

Analogously to Step 1 of Example 1, an ester compound (S)-3-(4-((5-(4-(5,6-dihydro-4H-1,3-oxazin-2-yl)phenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (430 mg) and 4-(5,6-dihydro-4H-1,3-oxazine)phenylboronic acid (1.2 eq.). Analogously to Step 2 of Example 1, the ester compound was then hydrolyzed to afford the title compound (S)-3-(4-((5-(4-(5,6-dihydro-4H-1,3-oxazin-2-yl)phenyl)-3-methylthiophen-2-yl)methoxy)phenyl)-2-ethoxypropionic acid (Example 19).

¹H NMR (DMSO-d₆, 400 MHz): 7.81 (d, 2H, J=6.8 Hz), 7.60 (d, 2H, J=8.4 Hz), 7.35 (s, 1H), 7.15 (d, 2H, J=6.8 Hz), 6.89 (d, 2H, J=6.8 Hz), 5.12 (s, 2H), 4.31 (s, 2H), 3.75 (br, 1H), 3.55 (m, 3H), 3.20 (m, 1H), 2.90 (m, 1H), 2.72 (m, 1H), 2.24 (s, 3H), 1.87 (m, 2H), and 0.99 (t, 3H, J=6.4 Hz).

### Example 20

### Preparation of (S)-2-ethoxy-3-(4-((3-methyl-5-(4-morpholinophenyl)thiophen-2-yl)methoxy)phenyl)propionic acid

Analogously to Step 1 of Example 1, an ester compound (S)-2-ethoxy-3-(4-((3-methyl-5-(4-morpholinophenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (340 mg) and 4-(morpholino)phenylboronic acid (1.2 eq.). Analogously to Step 2 of Example 1, the ester compound was then hydrolyzed to afford the title compound (S)-2-ethoxy-3-(4-((3-methyl-5-(4-morpholinophenyl)thiophen-2-yl)methoxy)phenyl)propionic acid (Example 20).

MS (ESI⁺) m/z 482.1 (M⁺¹).

### Example 21

### Preparation of (S)-2-ethoxy-3-(4-((3-methyl-5-(4-(2-methylthiazol-4-yl)phenyl)thiophen-2-yl)methoxy)phenyl)propionic acid

Analogously to Step 1 of Example 1, an ester compound (S)-2-ethoxy-3-(4-((3-methyl-5-(4-(2-methylthiazol-4-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (560 mg) and 4-(2-methylthiazole)phenylboronic acid (1.2 eq.). Analogously to Step 2 of Example 1, the ester compound was then hydrolyzed to afford the title compound (S)-2-ethoxy-3-(4-((3-methyl-5-(4-(2-methylthiazol-4-yl)phenyl)thiophen-2-yl)methoxy)phenyl)propionic acid (Example 21).

¹H NMR (DMSO-d₆, 400 MHz): 7.93 (m, 3H), 7.63 (d, 2H, J=7.2 Hz), 7.32 (s, 1H), 7.16 (d, 2H, J=8.0 Hz), 6.91 (d, 2H, J=7.6 Hz), 3.84 (m, 1H), 3.55 (m, 1H), 3.23 (m, 1H), 2.92 (m, 1H), 2.75 (m, 4H), 2.20 (s, 3H), and 1.00 (t, 3H, J=6.4 Hz).

### Example 22

### Preparation of (S)-2-ethoxy-3-(4-((3-methyl-5-(4-(1-methyl-6-oxo-1,4,5,6-tetrahydropyridazin-3-yl)phenyl)thiophen-2-yl)methoxy)phenyl)propionic acid

Analogously to Step 1 of Example 1, an ester compound (S)-2-ethoxy-3-(4-((3-methyl-5-(4-(1-methyl-6-oxo-1,4,5,6-tetrahydropyridazin-3-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (560 mg) and Compound 15b (1.2 eq.) synthesized in Preparation Example 20. Analogously to Step 2 of Example 1, the ester compound was then hydrolyzed to afford the title compound (S)-2-ethoxy-3-(4-((3-methyl-5-(4-(1-methyl-6-oxo-1,4,5,6-tetrahydropyridazin-3-yl)phenyl)thiophen-2-yl)methoxy)phenyl)propionic acid (Example 22).

¹H NMR (CDCl₃, 400 MHz): 7.72 (d, 2H, J=6.8 Hz), 7.58 (d, 2H, J=7.2 Hz), 7.18 (d, 2H, J=8.8 Hz), 7.12 (s, 1H), 6.93 (d, 2H, J=6.8 Hz), 5.08 (s, 2H), 4.06 (q, 1H, J=4.4 Hz), 3.58 (m, 1H), 3.47 (m, 4H), 3.07 (m, 1H), 2.98 (m, 3H), 2.60 (t, 2H, J=8.8 Hz), 2.29 (s, 3H), and 1.19 (t, 3H, J=7.2 Hz).

### Example 23

### Preparation of (S)-3-(4-((5-(4-(2H-benzo[b][1,4]oxazin-3-yl)phenyl)-3-methylthiophen-2-yl)methoxy)phenyl)-2-ethoxypropionic acid

Analogously to Step 1 of Example 1, an ester compound (S)-3-(4-((5-(4-(2H-benzo[b][1,4]oxazin-3-yl)phenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (320 mg) and 4-(2H-benzo[b][1,4]oxazine)phenylboronic acid (1.2 eq.). Analogously to Step 2 of Example 1, the ester compound was then hydrolyzed to afford the title compound (S)-3-(4-((5-(4-(2H-benzo[b][1,4]oxazin-3-yl)phenyl)-3-methylthiophen-2-yl)methoxy)phenyl)-2-ethoxypropionic acid (Example 23).

¹H NMR (DMSO-d₆, 400 MHz): 8.03 (d, 2H, J=7.2 Hz), 7.75 (d, 2H, J=7.6 Hz), 7.47 (s, 1H), 7.36 (d, 1H, J=7.6 Hz), 7.16 (d, 3H, J=8.0 Hz), 7.03 (t, 1H, J=7.6 Hz), 6.94 (d, 3H, J=8.0 Hz), 5.21 (t, 4H, J=4.0 Hz), 3.88 (m, 1H), 3.53 (m, 1H), 2.90 (m, 1H), 2.79 (m, 1H), 2.24 (s, 3H), and 1.04 (t, 3H, J=6.8 Hz).

### Example 24

### Preparation of (S)-3-(4-((5-(4-(1,2,3-thiadiazol-4-yl)phenyl)-3-methylthiophen-2-yl)methoxy)phenyl)-2-ethoxypropionic acid

Analogously to Step 1 of Example 1, an ester compound (S)-3-(4-((5-(4-(1,2,3-thiadiazol-4-yl)phenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (220 mg) and 4-(1,2,3-thiadiazole)phenylboronic acid (1.2 eq.). Analogously to Step 2 of Example 1, the ester compound was then hydrolyzed to afford the title compound (S)-3-(4-((5-(4-(1,2,3-thiadiazol-4-yl)phenyl)-3-methylthiophen-2-yl)methoxy)phenyl)-2-ethoxypropionic acid (Example 24).

MS (ESI⁺) m/z 481.1 (M⁺¹).

### Example 25

### Preparation of (S)-2-ethoxy-3-(4-((3-methyl-5-(4-(5-methyl-1,2,4-oxadiazol-3-yl)phenyl)thiophen-2-yl)methoxy)phenyl)propionic acid

Analogously to Step 1 of Example 1, an ester compound (S)-2-ethoxy-3-(4-((3-methyl-5-(4-(5-methyl-1,2,4-oxadiazol-3-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (430 mg) and Compound 16c (1.2 eq.) synthesized in Preparation Example 21. Analogously to Step 2 of Example 1, the ester compound was then hydrolyzed to afford the title compound (S)-2-ethoxy-3-(4-((3-methyl-5-(4-(5-methyl-1,2,4-oxadiazol-3-yl)phenyl)thiophen-2-yl)methoxy)phenyl)propionic acid (Example 25).

¹H NMR (CDCl₃, 400 MHz): 8.03 (d, 2H, J=8.8 Hz), 7.65 (d, 2H, J=6.4 Hz), 7.17 (m, 3H), 6.92 (d, 2H, J=8.8 Hz), 5.09 (s, 2H), 4.07 (q, 1H, J=4.4 Hz), 3.57 (m, 1H), 3.49 (m, 1H), 3.08 (m, 1H), 2.98 (m, 1H), 2.64 (s, 3H), 2.26 (s, 3H), and 1.19 (t, 3H, J=7.6 Hz).

### Example 26

### Preparation of (S)-2-ethoxy-3-(4-((3-methyl-5-(4-(5-methyl-1,3,4-oxadiazol-2-yl)phenyl)thiophen-2-yl)methoxy)phenyl)propionic acid

Analogously to Step 1 of Example 1, an ester compound (S)-2-ethoxy-3-(4-((3-methyl-5-(4-(5-methyl-1,3,4-oxadiazol-2-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (510 mg) and Compound 17b (1.2 eq.) synthesized in Preparation Example 22. Analogously to Step 2 of Example 1, the ester compound was then hydrolyzed to afford the title compound (S)-2-ethoxy-3-(4-((3-methyl-5-(4-(5-methyl-1,3,4-oxadiazol-2-yl)phenyl)thiophen-2-yl)methoxy)phenyl)propionic acid (Example 26).

¹H NMR (CDCl₃, 400 MHz): 7.99 (d, 2H, J=6.8 Hz), 7.66 (d, 2H, J=6.8 Hz), 7.18 (m, 3H), 6.92 (d, 2H, J=6.4 Hz), 5.09 (s, 2H), 4.07 (q, 1H, J=4.0 Hz), 3.61 (m, 1H), 3.48 (m, 1H), 3.07 (m, 1H), 2.98 (m, 1H), 2.61 (s, 3H), 2.32 (s, 3H), and 1.19 (t, 3H, J=7.2 Hz).

MS (ESI⁺) m/z 479.1 (M⁺¹).

### Example 27

### Preparation of (S)-2-ethoxy-3-(4-((3-methyl-5-(4-(5-(trifluoromethyl)-1,3,4-oxadiazol-2-yl)phenyl)thiophen-2-yl)methoxy)phenyl)propionic acid

Analogously to Step 1 of Example 1, an ester compound (S)-2-ethoxy-3-(4-((3-methyl-5-(4-(5-(trifluoromethyl)-1,3,4-oxadiazol-2-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (460 mg) and Compound 18b (1.2 eq.) synthesized in Preparation Example 23. Analogously to Step 2 of Example 1, the ester compound was then hydrolyzed to afford the title compound (S)-2-ethoxy-3-(4-((3-methyl-5-(4-(5-(trifluoromethyl)-1,3,4-oxadiazol-2-yl)phenyl)thiophen-2-yl)methoxy)phenyl)propionic acid (Example 27).

¹H NMR (DMSO-d₆, 400 MHz): 10.62 (br, 1H), 7.88 (d, 2H, J=8.0 Hz), 7.75 (d, 2H, J=8.4 Hz), 7.45 (s, 1H), 7.16 (d, 1H, J=8.4 Hz), 6.94 (d, 2H, J=8.8 Hz), 5.18 (s, 2H), 3.95 (q, 1H, J=4.8 Hz), 3.52 (m, 1H), 3.30 (m, 1H), 2.87 (m, 2H), 2.29 (s, 3H), and 1.04 (t, 3H, J=6.8 Hz).

MS (ESI⁺) m/z 533.1 (M⁺¹).

### Example 28

### Preparation of (S)-3-(4-((5-(4-(1,3,4-oxadiazol-2-yl)phenyl)-3-methylthiophen-2-yl)methoxy)phenyl)-2-ethoxypropionic acid

Analogously to Step 1 of Example 1, an ester compound (S)-3-(4-((5-(4-(1,3,4-oxadiazol-2-yl)phenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (460 mg) and Compound 19d (1.2 eq.) synthesized in Preparation Example 24. Analogously to Step 2 of Example 1, the ester compound was then hydrolyzed to afford the title compound (S)-3-(4-((5-(4-(1,3,4-oxadiazol-2-yl)phenyl)-3-methylthiophen-2-yl)methoxy)phenyl)-2-ethoxypropionic acid (Example 28).

MS (ESI⁺) m/z 465.1 (M⁺¹).

### Example 29

### Preparation of (S)-2-ethoxy-3-(4-((3-methyl-5-(4-(2-methyl-2H-tetrazol-5-yl)phenyl)thiophen-2-yl)methoxy)phenyl)propionic acid

Analogously to Step 1 of Example 1, an ester compound (S)-2-ethoxy-3-(4-((3-methyl-5-(4-(2-methyl-2,1-tetrazol-5-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (390 mg) and Compound 13b (1.2 eq.) synthesized in Preparation Example 14. Analogously to Step 2 of Example 1, the ester compound was then hydrolyzed to afford the title compound (S)-2-ethoxy-3-(4-((3-methyl-5-(4-(2-methyl-2H-tetrazol-5-yl)phenyl)thiophen-2-yl)methoxy)phenyl)propionic acid (Example 29).

¹H NMR (CDCl₃, 400 MHz): 8.11 (d, 2H, J=6.8 Hz), 7.67 (d, 2H, J=7.2 Hz), 7.17 (m, 3H), 6.93 (d, 2H, J=6.8 Hz), 5.09 (s, 2H), 4.38 (s, 3H), 4.07 (q, 1H, J=4.4 Hz), 3.58 (m, 1H), 3.49 (m, 1H), 3.08 (m, 1H), 2.98 (m, 1H), 2.27 (s, 3H), and 1.19 (t, 3H, J=7.2 Hz).

MS (ESI⁺) m/z 479.1 (M⁺¹).

### Example 30

### Preparation of (S)-2-ethoxy-3-(4-((3-methyl-5-(4-(1-methyl-1H-tetrazol-5-yl)phenyl)thiophen-2-yl)methoxy)phenyl)propionic acid

Analogously to Step 1 of Example 1, an ester compound (S)-2-ethoxy-3-(4-((3-methyl-5-(4-(1-methyl-1H-tetrazol-5-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (390 mg) and Compound 13b-1 (1.2 eq.) synthesized in Preparation Example 15. Analogously to Step 2 of Example 1, the ester compound was then hydrolyzed to afford the title compound (S)-2-ethoxy-3-(4-((3-methyl-5-(4-(1-methyl-1H-tetrazol-5-yl)phenyl)thiophen-2-yl)methoxy)phenyl)propionic acid (Example 30).

¹H NMR (CDCl₃, 400 MHz): 8.12 (d, 2H, J=6.8 Hz), 7.68 (d, 2H, J=7.2 Hz), 7.17 (m, 3H), 6.93 (d, 2H, J=6.8 Hz), 5.09 (s, 2H), 4.16 (s, 3H), 4.05 (q, 1H, J=4.4 Hz), 3.57 (m, 1H), 3.50 (m, 1H), 3.10 (m, 1H), 2.98 (m, 1H), 2.28 (s, 3H), and 1.20 (t, 3H, J=7.2 Hz).

### Example 31

### Preparation of (S)-2-ethoxy-3-(4-((5-(4-(2-isopropyl-2H-tetrazol-5-yl)phenyl)-3-methylthiophen-2-yl)methoxy)phenyl)propionic acid

Analogously to Step 1 of Example 1, an ester compound (S)-2-ethoxy-3-(4-((5-(4-(2-isopropyl-2H-tetrazol-5-yl)phenyl)-3-methylthiophen-2-yl)methoxy)phenyl)prop ionic acid ethyl ester was prepared from (S)-3-(4-((5-bromo-3-methylthiophen-2-yl)methoxy)phenyl)-2-ethoxypropionic acid ethyl ester (390 mg) and Compound 13d (1.2 eq.) synthesized in Preparation Example 16. Analogously to Step 2 of Example 1, the ester compound was then hydrolyzed to afford the title compound (S)-2-ethoxy-3-(4-((5-(4-(2-isopropyl-2H-tetrazol-5-yl)phenyl)-3-methylthiophen-2-yl)methoxy)phenyl)propionic acid (Example 31).

¹H NMR (CDCl₃, 400 MHz): 8.13 (d, 2H, J=7.2 Hz), 7.66 (d, 2H, J=6.8 Hz), 7.17 (m, 3H), 6.93 (d, 2H, J=6.4 Hz), 5.13 (m, 3H), 4.07 (q, 1H, J=4.0 Hz), 3.59 (m, 1H), 3.49 (m, 1H), 3.11 (m, 1H), 2.98 (m, 1H), 2.31 (s, 3H), 1.70 (d, 6H, J=3.6 Hz), and 1.16 (t, 3H, J=4.8 Hz).

### Example 32

### Preparation of (S)-2-ethoxy-3-(4-((5-(4-(2-(methoxymethyl)-2H-tetrazol-5-yl)phenyl)-3-methylthiophen-2-yl)methoxy)phenyl)propionic acid

Analogously to Step 1 of Example 1, an ester compound (S)-2-ethoxy-3-(4-((5-(4-(2-(methoxymethyl)-2H-tetrazol-5-yl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (500 mg) and Compound 13f (1.2 eq.) synthesized in Preparation Example 17. Analogously to Step 2 of Example 1, the ester compound was then hydrolyzed to afford the title compound (S)-2-ethoxy-3-(4-((5-(4-(2-(methoxymethyl)-2H-tetrazol-5-yl)phenyl)-3-methylthiophen-2-yl)methoxy)phenyl)propionic acid (Example 32).

MS (ESI⁺) m/z 509.2 (M⁺¹).

### Example 33

### Preparation of (S)-2-ethoxy-3-(4-((5-(4-(2-(hydroxymethyl)-2H-tetrazol-5-yl)phenyl)-3-methylthiophen-2-yl)methoxy)phenyl)propionic acid

Analogously to Step 1 of Example 1, an ester compound (S)-2-ethoxy-3-(4-((5-(4-(2-(hydroxymethyl)-2H-tetrazol-5-yl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (520 mg) and Compound 13g (1.2 eq.) synthesized in Preparation Example 18. Analogously to Step 2 of Example 1, the ester compound was then hydrolyzed to afford the title compound (S)-2-ethoxy-3-(4-((5-(4-(2-(hydroxymethyl)-2H-tetrazol-5-yl)phenyl)-3-methylthiophen-2-yl)methoxy)phenyl)propionic acid (Example 33).

MS (ESI⁺) m/z 495.1 (M⁺¹).

### Example 34

### Preparation of (S)-3-(4-((5-(4-(4,5-dimethyloxazol-2-yl)phenyl)-3-methylthiophen-2-yl)methoxy)phenyl)-2-ethoxypropionic acid

Analogously to Step 1 of Example 1, an ester compound (S)-3-(4-((5-(4-(4,5-dimethyloxazol-2-yl)phenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-α-ethoxypropionic acid ethyl ester (430 mg) and Compound 20e (1.2 eq.) synthesized in Preparation Example 25. Analogously to Step 2 of Example 1, the ester compound was then hydrolyzed to afford the title compound (S)-3-(4-((5-(4-(4,5-dimethyloxazol-2-yl)phenyl)-3-methylthiophen-2-yl)methoxy)phenyl)-2-ethoxypropionic acid (Example 34).

¹H NMR (CDCl₃, 400 MHz): 7.98 (d, 2H, J=8.0 Hz), 7.61 (d, 2H, J=8.0 Hz), 7.17 (s, 1H), 7.15 (d, 2H, J=8.0 Hz), 6.92 (d, 2H, J=8.0 Hz), 5.08 (s, 2H), 4.07 (q, 1H, J=4.4 Hz), 3.58 (m, 1H), 3.50 (m, 1H), 3.11 (m, 1H), 3.07 (m, 1H), 2.31 (s, 3H), 2.25 (s, 3H), 2.17 (s, 3H), and 1.19 (t, 3H, J=7.2 Hz).

### Example 35

### Preparation of (S)-2-ethoxy-3-(4-((5-(4-(5-(hydroxymethyl)isoxazol-3-yl)phenyl)-3-methylthiophen-2-yl)methoxy)phenyl)propionic acid

Analogously to Step 1 of Example 1, an ester compound (S)-2-ethoxy-3-(4-((5-(4-(5-(hydroxymethyl)isoxazol-3-yl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (600 mg) and Compound 11h (1.2 eq.) synthesized in Preparation Example 7. Analogously to Step 2 of Example 1, the ester compound was then hydrolyzed to afford the title compound (S)-2-ethoxy-3-(4-((5-(4-(5-(hydroxymethyl)isoxazol-3-yl)phenyl)-3-methylthiophen-2-yl)methoxy)phenyl)propionic acid (Example 35).

¹H NMR (DMSO-d₆, 400 MHz): 7.88 (d, 2H, J=6.8 Hz), 7.72 (d, 2H, J=6.8 Hz), 7.41 (s, 1H), 7.16 (d, 2H, J=7.6 Hz), 6.94 (m, 3H), 5.15 (s, 2H), 4.60 (s, 2H), 3.64 (br, 1H), 3.52 (m, 1H), 3.19 (m, 1H), 2.87 (m, 1H), 2.69 (m, 1H), 2.23 (s, 3H), and 0.99 (t, 3H, J=7.2 Hz).

### Example 36

### Preparation of (S)-2-ethoxy-3-(4-((5-(4-(5-(methoxymethyl)isoxazol-3-yl)phenyl)-3-methylthiophen-2-yl)methoxy)phenyl)propionic acid

Analogously to Step 1 of Example 1, an ester compound (S)-2-ethoxy-3-(4-((5-(4-(5-(methoxymethyl)isoxazol-3-yl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (630 mg) and Compound 11g (1.2 eq.) synthesized in Preparation Example 8. Analogously to Step 2 of Example 1, the ester compound was then hydrolyzed to afford the title compound (S)-2-ethoxy-3-(4-((5-(4-(5-(methoxymethyl)isoxazol-3-yl)phenyl)-3-methylthiophen-2-yl)methoxy)phenyl)propionic acid (Example 36).

¹H NMR (DMSO-d₆, 400 MHz): 7.90 (d, 2H, J=8.0 Hz), 7.74 (d, 2H, J=8.0 Hz), 7.42 (s, 1H), 7.16 (d, 2H, J=8.4 Hz), 7.09 (s, 1H), 6.93 (d, 2H, J=8.4 Hz), 5.21 (s, 2H), 4.59 (s, 2H), 3.90 (q, 1H, J=4.4 Hz), 3.53 (m, 1H), 3.36 (s, 3H), 3.29 (m, 1H), 2.90 (m, 1H), 2.79 (m, 1H), 2.19 (s, 3H), and 1.03 (t, 3H, J=6.8 Hz).

MS (ESI⁺) m/z 508.1 (M⁺¹).

### Example 37

### Preparation of (S)-5-(4-(5-((4-(2-carbonyl-2-ethoxyethyl)phenoxy)methyl)-4-methylthiophen-2-yl phenyl)isoxazole-3-carboxylic acid

Analogously to Step 1 of Example 1, an ester compound (S)-5-(4-(5-((4-(2-carbonyl-2-ethoxyethyl)phenoxy)methyl)-4-methylthiophen-2-yl)phenyl)isoxazole-3-carboxylate ethyl ester was prepared from (S)-3-(4-((5-bromo-3-methylthiophen-2-yl)methoxy)phenyl)-2-ethoxypropionic acid ethyl ester (530 mg) and Compound 12h (1.2 eq.) synthesized in Preparation Example 10. Analogously to Step 2 of Example 1, the ester compound was then hydrolyzed to afford the title compound (S)-5-(4-(5-((4-(2-carbonyl-2-ethoxyethyl)phenoxy)methyl)-4-methylthiophen-2-yl)phenyl)isoxazole-3-carboxylic acid (Example 37).

¹H NMR (CDCl₃+DMSO-d₆, 400 MHz): 7.70 (m, 2H), 7.57 (d, 2H, J=6.4 Hz), 7.12 (d, 2H, J=8.8 Hz), 7.07 (s, 1H), 6.82 (m, 3H), 5.00 (s, 2H), 3.87 (q, 1H, J=4.4 Hz), 3.57 (m, 1H), 3.26 (m, 1H), 2.91 (m, 1H), 2.86 (m, 1H), 2.49 (m, 1H), 2.18 (s, 3H), and 1.03 (t, 3H, J=5.6 Hz).

### Example 38

### Preparation of (S)-2-ethoxy-3-(4-((3-methyl-5-(4-(3-(methylcarbamoyl)isoxazol-5-yl)phenyl)thiophen-2-yl)methoxy)phenyl)propionic acid

Analogously to Step 1 of Example 1, an ester compound (S)-2-ethoxy-3-(4-((3-methyl-5-(4-(3-(methylcarbamoyl)isoxazol-5-yl)phenyl)thiophen-2-yl)methoxy)phenyl)prop ionic acid ethyl ester was prepared from (S)-3-(4-((5-bromo-3-methylthiophen-2-yl)methoxy)phenyl)-2-ethoxypropionic acid ethyl ester (320 mg) and Compound 12m (1.2 eq.) synthesized in Preparation Example 13. Analogously to Step 2 of Example 1, the ester compound was then hydrolyzed to afford the title compound (S)-2-ethoxy-3-(4-((3-methyl-5-(4-(3-(methylcarbamoyl)isoxazol-5-yl)phenyl)thiophen-2-yl)methoxy)phenyl)propionic acid (Example 38).

MS (ESI⁺) m/z 521.1 (M⁺¹).

### Example 39

### Preparation of (S)-2-ethoxy-3-(4-((5-(4-(3-(hydroxymethyl)isoxazol-5-yl)phenyl)-3-methylthiophen-2-yl)methoxy)phenyl)propionic acid

Analogously to Step 1 of Example 1, an ester compound (S)-2-ethoxy-3-(4-((5-(4-(3-(hydroxymethyl)isoxazol-5-yl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (610 mg) and Compound 12i (1.2 eq.) synthesized in Preparation Example 11. Analogously to Step 2 of Example 1, the ester compound was then hydrolyzed to afford the title compound (S)-2-ethoxy-3-(4-((5-(4-(3-(hydroxymethyl)isoxazol-5-yl)phenyl)-3-methylthiophen-2-yl)methoxy)phenyl)propionic acid (Example 39).

MS (ESI⁺) m/z 494.1 (M⁺¹).

### Example 40

### Preparation of (S)-2-ethoxy-3-(4-((5-(4-(3-(methoxymethyl)isoxazol-5-yl)phenyl)-3-methylthiophen-2-yl)methoxy)phenyl)propionic acid

Analogously to Step 1 of Example 1, an ester compound (S)-2-ethoxy-3-(4-((5-(4-(3-(methoxymethyl)isoxazol-5-yl)phenyl)-3-methylthiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (540 mg) and Compound 12j (1.2 eq.) synthesized in Preparation Example 12. Analogously to Step 2 of Example 1, the ester compound was then hydrolyzed to afford the title compound (S)-2-ethoxy-3-(4-((5-(4-(3-(methoxymethyl)isoxazol-5-yl)phenyl)-3-methylthiophen-2-yl)methoxy)phenyl)propionic acid (Example 40).

MS (ESI⁺) m/z 508.1 (M⁺¹).

### Example 41

### Preparation of (S)-2-ethoxy-3-(4-((3-methyl-5-(4-(3-methylisoxazol-5-yl)phenyl)thiophen-2-yl)methoxy)phenyl)propionic acid

Analogously to Step 1 of Example 1, an ester compound (S)-2-ethoxy-3-(4-((3-methyl-5-(4-(3-methylisoxazol-5-yl)phenyl)thiophen-2-yl)methoxy)phenyl)propionic acid ethyl ester was prepared from (S)-3-(4-((5-bromo-3-methylthiophen-2-yl)methoxy)phenyl)-2-ethoxypropionic acid ethyl ester (5.0 g) and Compound 12c (1.2 eq.) synthesized in Preparation Example 9. Analogously to Step 2 of Example 1, the ester compound was then hydrolyzed to afford the title compound (S)-2-ethoxy-3-(4-((3-methyl-5-(4-(3-methylisoxazol-5-yl)phenyl)thiophen-2-yl)methoxy)phenyl)propionic acid (Example 41).

¹H NMR (CDCl₃, 400 MHz): 7.73 (d, 2H, J=8.4 Hz), 7.62 (d, 2H, J=8.4 Hz), 7.25 (d, 2H, J=8.0 Hz), 7.15 (s, 1H), 6.93 (d, 2H, J=8.4 Hz), 6.34 (s, 1H), 5.08 (s, 2H), 4.07 (q, 1H, J=4.4 Hz), 3.60 (m, 1H), 3.49 (m, 1H), 3.11 (m, 1H), 3.07 (m, 1H), 2.36 (s, 3H), 2.30 (s, 3H), and 1.19 (t, 3H, J=7.2 Hz).

MS (ESI⁺) m/z 478.1 (M⁺¹).

### Example 42

### Preparation of lithium (S)-2-ethoxy-3-(4-((3-methyl-5-(4-(3-methylisoxazol-5-yl)phenyl)thiophen-2-yl)methoxy)phenyl)propanoate

(S)-2-ethoxy-3-(4-((3-methyl-5-(4-(3-methylisoxazol-5-yl)phenyl)thiophen-2-yl)methoxy)phenyl)propionic acid (3.0 g) synthesized in Example 41 was dissolved in a mixture of ethyl acetate (10 mL) and acetone (1 mL), which was followed by addition of 2-ethylhexanoic acid lithium salt (1.2 eq.) and stirring at room temperature for 1 hour, as disclosed in Reaction Scheme 23. The resulting white solids were filtered, sequentially washed with ethyl acetate (5 mL), ethyl ether (5 mL) and hexane (5 mL), and dried under vacuum to afford the title compound lithium (S)-2-ethoxy-3-(4-((3-methyl-5-(4-(3-methylisoxazol-5-yl)phenyl)thiophen-2-yl)methoxy)phenyl)propanoate (Example 42). Yield: 90%.

¹H NMR (DMSO-d₆, 400 MHz): 7.82 (d, 2H, J=8.4 Hz), 7.73 (d, 2H, J=8.4 Hz), 7.41 (s, 1H), 7.12 (d, 2H, J=8.4 Hz), 6.89 (m, 3H), 5.14 (s, 2H), 3.55 (m, 1H), 3.47 (m, 1H), 3.46 (bs, 1H), 3.08 (m, 1H), 2.83 (m, 1H), 2.26 (s, 3H), 2.24 (s, 3H), and 0.97 (t, 3H, J=7.2 Hz).

MS (ESI⁺) m/z 478.1 (M⁺¹), 484.1 (M+Li).

### Example 43

### Preparation of sodium (S)-2-ethoxy-3-(4-((3-methyl-5-(4-(3-methylisoxazol-5-yl)phenyl)thiophen-2-yl)methoxy)phenyl)propanoate

(S)-2-ethoxy-3-(4-((3-methyl-5-(4-(3-methylisoxazol-5-yl)phenyl)thiophen-2-yl)methoxy)phenyl)propionic acid (2.0 g) synthesized in Example 41 was dissolved in a mixture of ethyl acetate (7 mL) and acetone (1 mL), which was followed by addition of 2-ethylhexanoic acid sodium salt (1.2 eq.) and stirring at room temperature for 1 hour, as disclosed in Reaction Scheme 23. The resulting white solids were filtered, sequentially washed with ethyl acetate (5 mL), ethyl ether (5 mL) and hexane (5 mL), and dried under vacuum to afford the title compound sodium (S)-2-ethoxy-3-(4-((3-methyl-5-(4-(3-methylisoxazol-5-yl)phenyl)thiophen-2-yl)methoxy)phenyl)propanoate (Example 43). Yield: 90%.

¹H NMR (DMSO-d₆, 400 MHz): 7.81 (d, 2H, J=8.4 Hz), 7.73 (d, 2H, J=8.0 Hz), 7.41 (s, 1H), 7.12 (d, 2H, J=8.4 Hz), 6.87 (m, 3H), 5.14 (s, 2H), 3.55 (m, 1H), 3.47 (m, 1H), 3.08 (m, 1H), 2.83 (m, 1H), 2.26 (s, 3H), 2.24 (s, 3H), and 0.99 (t, 3H, J=7.2 Hz).

MS (ESI⁺) m/z 478.1 (M⁺¹), 500.1 (M+Na).

### Example 44

### Preparation of potassium (S)-2-ethoxy-3-(4-((3-methyl-5-(4-(3-methylisoxazol-5-yl)phenyl)thiophen-2-yl)methoxy)phenyl)propanoate

(S)-2-ethoxy-3-(4-((3-methyl-5-(4-(3-methylisoxazol-5-yl)phenyl)thiophen-2-yl)methoxy)phenyl)propionic acid (4.0 g) synthesized in Example 41 was dissolved in a mixture of ethyl acetate (15 mL) and acetone (2 mL), which was followed by addition of 2-ethylhexanoic acid potassium salt (1.2 eq.) and stirring at room temperature for 1 hour, as disclosed in Reaction Scheme 23. The resulting white solids were filtered, sequentially washed with ethyl acetate (7 mL), ethyl ether (5 mL) and hexane (10 mL), and dried under vacuum to afford the title compound potassium (S)-2-ethoxy-3-(4-((3-methyl-5-(4-(3-methylisoxazol-5-yl)phenyl)thiophen-2-yl)methoxy)phenyl)propanoate (Example 44). Yield: 90%.

¹H NMR (DMSO-d₆, 400 MHz): 7.83 (d, 2H, J=8.4 Hz), 7.75 (d, 2H, J=8.0 Hz), 7.43 (s, 1H), 7.12 (d, 2H, J=8.4 Hz), 6.89 (s, 1H), 6.97 (d, 2H, J=8.4 Hz), 5.14 (s, 2H), 3.55 (m, 1H), 3.47 (m, 1H), 3.08 (m, 1H), 2.83 (m, 1H), 2.27 (s, 3H), 2.24 (s, 3H), and 0.96 (t, 3H, J=7.2 Hz).

MS (ESI⁺) m/z 478.1 (M⁺¹), 516.1 (M+K).

### Example 45

### Preparation of (S)-2-ethoxy-3-(4-((5-(4-(3-methylisoxazol-5-yl)phenyl)furan-2-yl)methoxy)phenyl)propionic acid

Analogously to Step 1 of Example 1, (S)-2-ethoxy-3-(4-((5-(4-(3-methylisoxazol-5-yl)phenyl)furan-2-yl)methoxy)phenyl)propionic acid ethyl ester (500 mg) was prepared from (S)-ethyl 3-(4-((5-bromofuran-2-yl)methoxy)phenyl)-2-ethoxypropanoate (Compound 8a, 1 g, 2.51 mmol) synthesized in Preparation Example 3 and Compound 12c (1.2 eq.) synthesized in Preparation Example 9. Analogously to Step 2 of Example 1, the ester compound was then hydrolyzed to afford the title compound (S)-2-ethoxy-3-(4-((5-(4-(3-methylisoxazol-5-yl)phenyl)furan-2-yl)methoxy)phenyl)propionic acid (Example 45).

MS (ESI⁺) m/z 448.1 (M⁺¹).

### Example 46

### Preparation of (S)-2-ethoxy-3-(4-((3-methyl-5-(4-(3-methylisoxazol-5-yl)phenyl)furan-2-yl)methoxy)phenyl)propionic acid

Analogously to Step 1 of Example 1, (S)-2-ethoxy-3-(4-((3-methyl-5-(4-(3-methylisoxazol-5-yl)phenyl)furan-2-yl)methoxy)phenyl)propionic acid ethyl ester (300 mg) was prepared from (S)-3-(4-((5-bromo-3-methylfuran-2-yl)methoxy)phenyl)-2-ethoxypropionic acid ethyl ester (Compound 9a, 1.0 g, 2.43 mmol) synthesized in Preparation Example 4 and Compound 12c (1.2 eq.) synthesized in Preparation Example 9. Analogously to Step 2 of Example 1, the ester compound was then hydrolyzed to afford the title compound (S)-2-ethoxy-3-(4-((3-methyl-5-(4-(3-methylisoxazol-5-yl)phenyl)furan-2-yl)methoxy)phenyl)propionic acid (Example 46).

MS (ESI⁺) m/z 462.1 (M⁺¹).

### Example 47

### Preparation of (S)-2-ethoxy-3-(4-((4-methyl-2-(4-(3-methylisoxazol-5-yl)phenyl)thiazol-5-yl)methoxy)phenyl)propionic acid

Analogously to Step 1 of Example 1, (S)-2-ethoxy-3-(4-((4-methyl-2-(4-(3-methylisoxazol-5-yl)phenyl)thiazol-5-yl)methoxy)phenyl)propionic acid ethyl ester (300 mg) was prepared from (S)-ethyl-3-(4-((2-bromo-4-methylthiazol-2-yl)methoxy)phenyl)-2-ethoxypropanoate (Compound 10a, 1.0 g, 2.33 mmol) synthesized in Preparation Example 5 and Compound 12c (1.2 eq.) synthesized in Preparation Example 9. Analogously to Step 2 of Example 1, the ester compound was then hydrolyzed to afford the title compound (S)-2-ethoxy-3-(4-((4-methyl-2-(4-(3-methylisoxazol-5-yl)phenyl)thiazol-5-yl)methoxy)phenyl)propionic acid (Example 47).

¹H NMR (DMSO-d₆, 400 MHz): 8.01 (d, 2H, J=8.8 Hz), 7.92 (d, 2H, J=8.0 Hz), 7.15 (d, 2H, J=8.4 Hz), 6.97 (s, 1H), 6.92 (d, 2H, J=8.8 Hz), 5.28 (s, 2H), 3.84 (m, 1H), 3.52 (m, 1H), 3.24 (m, 1H), 2.86 (m, 11H), 3.74 (m, 1H), 2.42 (s, 3H), 2.29 (s, 3H), and 1.03 (t, 3H, J=7.2 Hz).

MS (ESI⁺) m/z 479.1 (M⁺¹).

### Example 48

### Preparation of (S)-3-(4-((5-(4-(5-tert-butylisoxazol-3-yl)phenyl)-3-methylthiophen-2-yl)methoxy)phenyl)-2-ethoxypropionic acid

Analogously to Step 1 of Example 1, an ester compound (S)-3-(4-((5-(4-(5-tert-butylisoxazol-3-yl)phenyl)-3-methylthiophen-2-yl)methoxy)phenyl)-2-ethoxypropionic acid ethyl ester was prepared from (S)-3-(4-((5-bromo-3-methylthiophen-2-yl)methoxy)phenyl)-2-ethoxypropionic acid ethyl ester (9.0 g) and Compound 11d (1.2 eq.) synthesized in Preparation Example 6. Analogously to Step 2 of Example 1, the ester compound was then hydrolyzed to afford the title compound (S)-3-(4-((5-(4-(5-tert-butylisoxazol-3-yl)phenyl)-3-methylthiophen-2-yl)methoxy)phenyl)-2-ethoxypropionic acid (Example 48).

¹H NMR (DMSO-d₆, 400 MHz): 7.87 (d, 2H, J=7.8 Hz), 7.72 (d, 2H, J=7.8 Hz), 7.42 (s, 1H), 7.15 (d, 2H, J=7.8 Hz), 6.93 (d, 2H, J=7.8 Hz), 6.84 (s, 1H), 5.17 (s, 1H), 3.93 (t, 1H, J=6.0 Hz), 3.49 (m, 1H), 3.29 (m, 1H), 2.86 (m, 2H), 2.39 (s, 3H), 1.34 (s, 9H), and 1.03 (t, 3H, J=7.2 Hz).

MS (ESI⁺) m/z 520.1 (M⁺¹).

### Example 49

### Preparation of lithium (S)-3-(4-((5-(4-(5-tert-butylisoxazol-3-yl)phenyl)-3-methylthiophen-2-yl)methoxy)phenyl)-2-ethoxypropanoate

(S)-3-(4-((5-(4-(5-tert-butylisoxazol-3-yl)phenyl)-3-methylthiophen-2-yl)methoxy)phenyl)-2-ethoxypropionic acid (3.0 g) synthesized in Example 48 was dissolved in a mixture of ethyl acetate (10 mL) and acetone (1 mL), which was followed by addition of 2-ethylhexanoic acid lithium salt (1.2 eq.) and stirring at room temperature for 1 hour, as disclosed in Reaction Scheme 23. The resulting white solids were filtered, sequentially washed with ethyl acetate (5 mL), ethyl ether (5 mL) and hexane (5 mL), and dried under vacuum to afford the title compound lithium (S)-3-(4-((5-(4-(5-tert-butylisoxazol-3-yl)phenyl)-3-methylthiophen-2-yl)methoxy)phenyl)-2-ethoxypropanoate (Example 49). Yield: 90%.

¹H NMR (DMSO-d₆, 400 MHz): 7.88 (d, 2H, J=8.4 Hz), 7.71 (d, 2H, J=8.0 Hz), 7.40 (s, 1H), 7.11 (d, 2H, J=7.8 Hz), 6.87 (d, 2H, J=7.8 Hz), 6.82 (s, 1H), 5.16 (s, 1H), 3.56 (m, 1H), 3.47 (m, 1H), 3.09 (m, 1H), 2.81 (m, 1H), 2.59 (m, 1H), 2.24 (s, 3H), 1.34 (s, 9H), and 0.97 (t, 3H, J=7.2 Hz).

MS (ESI⁺) m/z 520.1 (M⁺¹), 526.1 (M+Li).

### Example 50

### Preparation of sodium (S)-3-(4-((5-(4-(5-tert-butylisoxazol-3-yl)phenyl)-3-methylthiophen-2-yl)methoxy)phenyl)-2-ethoxypropanoate

(S)-3-(4-((5-(4-(5-tert-butylisoxazol-3-yl)phenyl)-3-methylthiophen-2-yl)methoxy)phenyl)-2-ethoxypropionic acid (3.0 g) synthesized in Example 48 was dissolved in a mixture of ethyl acetate (10 mL) and acetone (1 mL), which was followed by addition of 2-ethylhexanoic acid sodium salt (1.2 eq.) and stirring at room temperature for 1 hour, as disclosed in Reaction Scheme 23. The resulting white solids were filtered, sequentially washed with ethyl acetate (5 mL), ethyl ether (5 mL) and hexane (5 mL), and dried under vacuum to afford the title compound sodium (S)-3-(4-((5-(4-(5-tert-butylisoxazol-3-yl)phenyl)-3-methylthiophen-2-yl)methoxy)phenyl)-2-ethoxypropanoate (Example 50). Yield: 90%.

¹H NMR (DMSO-d₆, 400 MHz): 7.85 (d, 2H, J=8.4 Hz), 7.72 (d, 2H, J=8.0 Hz), 7.40 (s, 1H), 7.11 (d, 2H, J=7.8 Hz), 6.87 (d, 2H, J=7.8 Hz), 6.82 (s, 1H), 5.16 (s, 1H), 3.56 (m, 1H), 3.47 (m, 1H), 3.09 (m, 1H), 2.81 (m, 1H), 2.59 (m, 1H), 2.24 (s, 3H), 1.35 (s, 9H), and 0.97 (t, 3H, J=7.2 Hz).

MS (ESI⁺) 520.1 (M⁺¹), 542.1 (M+Li).

### Example 51

### Preparation of potassium (S)-2-ethoxy-3-(4-((3-methyl-5-(4-(3-methylisoxazol-5-yl)phenyl)thiophen-2-yl)methoxy)phenyl)propanoate

(S)-3-(4-((5-(4-(5-tert-butylisoxazol-3-yl)phenyl)-3-methylthiophen-2-yl)methoxy)phenyl)-2-ethoxypropionic acid (4.0 g) synthesized in Example 48 was dissolved in a mixture of ethyl acetate (15 mL) and acetone (2 mL), which was followed by addition of 2-ethylhexanoic acid potassium salt (1.2 eq.) and stirring at room temperature for 1 hour, as disclosed in Reaction Scheme 23. The resulting white solids were filtered, sequentially washed with ethyl acetate (7 mL), ethyl ether (5 mL) and hexane (10 mL), and dried under vacuum to afford the title compound potassium (S)-2-ethoxy-3-(4-((3-methyl-5-(4-(3-methylisoxazol-5-yl)phenyl)thiophen-2-yl)methoxy)phenyl)propanoate (Example 51). Yield: 90%.

¹H NMR (DMSO-d₆, 400 MHz): 7.86 (d, 2H, J=8.4 Hz), 7.71 (d, 2H, J=8.0 Hz), 7.40 (s, 1H), 7.12 (d, 2H, J=8.4 Hz), 6.88 (d, 2H, J=8.4 Hz), 6.82 (s, 1H), 5.15 (s, 2H), 3.55 (m, 1H), 3.49 (m, 1H), 3.08 (m, 1H), 2.81 (m, 1H), 2.23 (s, 3H), 1.34 (s, 9H), and 0.96 (t, 3H, J=7.2 Hz).

MS (ESI⁺) m/z 520.1 (M⁺¹), 558.1 (M+K).

### Example 52

### Preparation of (S)-3-(4-((5-(4-(5-tert-butylisoxazol-3-yl)phenyl)furan-2-yl)methoxy)phenyl)-2-ethoxypropionic acid

Analogously to Step 1 of Example 1, (S)-3-(4-((5-(4-(5-tert-butylisoxazol-3-yl)phenyl)furan-2-yl)methoxy)phenyl)-2-ethoxypropionic acid ethyl ester (400 mg) was prepared from (S)-ethyl 3-(4-((5-bromofuran-2-yl)methoxy)phenyl)-2-ethoxypropanoate (Compound 8a, 1.0 g, 2.52 mmol) synthesized in Preparation Example 3 and Compound 1d (1.2 eq.) synthesized in Preparation Example 6. Analogously to Step 2 of Example 1, the ester compound was then hydrolyzed to afford the title compound (S)-3-(4-((5-(4-(5-tert-butylisoxazol-3-yl)phenyl)furan-2-yl)methoxy)phenyl)-2-ethoxypropionic acid (Example 52).

MS (ESI⁺) m/z 490.1 (M⁺¹).

### Example 53

### Preparation of (S)-3-(4-((5-(4-(5-tert-butylisoxazol-3-yl)phenyl)-3-methylfuran-2-yl)methoxy)phenyl)-2-ethoxypropionic acid

Analogously to Step 1 of Example 1, (S)-3-(4-((5-(4-(5-tert-butylisoxazol-3-yl)phenyl)-3-methylfuran-2-yl)methoxy)phenyl)-2-ethoxypropionic acid ethyl ester (300 mg) was prepared from (S)-3-(4-((5-bromo-3-methylfuran-2-yl)methoxy)phenyl)-2-ethoxypropionic acid ethyl ester (Compound 9a, 1.0 g, 2.43 mmol) synthesized in Preparation Example 4 and Compound 11d (1.2 eq.) synthesized in Preparation Example 6. Analogously to Step 2 of Example 1, the ester compound was then hydrolyzed to afford the title compound (S)-3-(4-((5-(4-(5-tert-butylisoxazol-3-yl)phenyl)-3-methylfuran-2-yl)methoxy)phenyl)-2-ethoxypropionic acid (Example 53).

MS (ESI⁺) m/z 504.1 (M⁺¹).

### Example 54

### Preparation of (S)-3-(4-((2-(4-(5-tert-butylisoxazol-3-yl)phenyl)-4-methylthiazol-5-VI)methoxy)phenyl)-2-ethoxypropionic acid

Analogously to Step 1 of Example 1, (S)-3-(4-((2-(4-(5-tert-butylisoxazol-3-yl)phenyl)-4-methylthiazol-5-yl)methoxy)phenyl)-2-ethoxypropionic acid ethyl ester (300 mg) was prepared from (S)-ethyl 3-(4-((2-bromo-4-methylthiazol-2-yl)methoxy)phenyl)-2-ethoxypropanoate (Compound 10a, 1.0 g, 2.33 mmol) synthesized in Preparation Example 5 and Compound 12c (1.2 eq.) synthesized in Preparation Example 9. Analogously to Step 2 of Example 1, the ester compound was then hydrolyzed to afford the title compound (S)-3-(4-((2-(4-(5-tert-butylisoxazol-3-yl)phenyl)-4-methylthiazol-5-yl)methoxy)phenyl)-2-ethoxypropionic acid (Example 54).

MS (ESI⁺) m/z 521.1 (M⁺¹).

### Experimental Example 1

### Effects of Inventive Compounds on Activation of PPAR-α and -γ

1. Materials and Methods

Following induction of transient intracellular expression of PPAR-α and -γ, an ability of the inventive compounds to induce transactivation of PPARs via activation of each PPAR was evaluated as an efficacy of the compound (transactivation assay).

For this assay, the African green monkey kidney cell line CV-1 (CCL-70, ATCC) was used as a test cell line, and PPAR-α and -γ were murine- and human-derived PPARs. Samples used were compounds prepared in Examples 19, 21, 23, 25, 26, 27, 31, 33, 34, 36, 38, 40, 42, 43 and 50. As a positive control drug, 3-4-[2-(2-phenyl-4-methyl-1,3-oxazole)ethyloxy]phenyl-(2S)-[(1-methyl-3-oxo-3-phenyl)propenyl]aminopropionic acid was used that is a PPAR-α or -γ agonist which was once under development and whose clinical trials and studies were suspended at phase III. A chimeric receptor was adopted to circumvent the probable interference due to endogenous receptor activation (Jian-Shen Q. et al., Mol Cell Biol (1995) 15(3):1817-1825). The chimeric receptor was constructed as a fusion of a PPAR-α or -γ ligand-binding domain with a DNA-binding domain of GAL4 which is a yeast transactivator.

The CV-1 cells were transiently transfected with each of chimeric receptor-expressing DNA constructs and each of DNA constructs comprising 5 copies of the GAL4 DNA-binding domain and capable of inducing expression of firefly luciferase or Renilla luciferase using a Lipofectamine Plus reagent (Invitrogen, USA). After transfection for 3 hours, the culture media were replaced with DMEM containing the above samples and 10% fetal bovine serum. 24 hours later, the firefly luciferase activity and Renilla luciferase activity were continuously assayed while adding an equal amount of a dual luciferase assay reagent (Promega, USA) to the cell-containing media. The transfection efficiency was normalized against Renilla luciferase activity (Motomura W. et al., Int J Cancer (2004) 108(1):41-6). The PPAR-α and -γ activity was determined by calculating Relative Response % to maximum effects of the positive control drug, and conducting multiple dose evaluation of the inventive compounds to calculate EC₅₀, which is the concentration of a drug which produces 50% activation relative to maximum effects of the inventive compounds, by nonlinear regression analysis.

2. Experimental Results

Representative compounds of the present invention exhibited EC₅₀ of 400 to 6000 nM for human PPAR-α and EC₅₀ of 7 to 1000 nM for human PPAR-γ (see Table 1). The maximum response of the inventive compounds for human PPAR-γ was found to be a 15 to 80% level of the positive control drug that causes 100% activation of PPAR-γ. That is, the compounds of formula 1 in accordance with the present invention were identified as drugs which activate PPAR-γ even at a low concentration, but exhibit a relatively low responsiveness as compared to the positive control drug inducing 100% activation and have higher activity for PPAR-γ than for PPAR-α. Therefore, a pharmaceutical composition comprising the compound of the present invention can be effectively used as a PPAR agonist that is expected to exhibit hypoglycemic, hypolipidemic and insulin resistance-reducing effects simultaneously with decreased adverse side effects of the drug.

TABLE 1
EC₅₀ of representative compounds on PPAR-α and -γ

| | Human | | | | Mouse | | | |
|---|---|---|---|---|---|---|---|---|
| | PPAR-α | | PPAR-γ | | PPAR-α | | PPAR-γ | |
| Ex. | | Max | | Max | | Max | | Max |
| No. | EC₅₀ | % | EC₅₀ | % | EC₅₀ | % | EC₅₀ | % |
| 25 | 5.244 | 63 | 0.095 | 48 | 0.443 | 35 | 0.295 | 48 |
| 41 | 3.154 | 23 | 0.062 | 30 | 6.160 | 19 | 0.142 | 34 |
| 19 | 6.568 | 44 | 0.045 | 43 | 6.826 | 19 | 0.281 | 43 |
| 48 | 2.831 | 22 | 0.007 | 27 | 12.149 | 25 | 0.031 | 24 |
| 21 | ND | 0 | 0.472 | 15 | ND | 4 | 0.883 | 18 |
| 36 | 2.304 | 25 | 0.041 | 37 | 4.300 | 21 | 0.354 | 48 |
| 34 | 0.476 | 31 | 0.018 | 75 | 3.442 | 31 | 0.176 | 81 |
| 29 | 1.231 | 52 | 0.049 | 66 | 0.381 | 31 | 0.118 | 48 |
| 23 | 2.008 | 44 | 0.065 | 83 | 0.105 | 43 | 0.165 | 67 |
| 24 | 1.402 | 19 | 0.032 | 45 | 4.926 | 23 | 0.084 | 38 |
| 38 | 5.143 | 51 | 0.1 | 68 | 5.783 | 49 | 0.180 | 81 |
| 40 | ND | 7 | 0.019 | 31 | ND | 5 | 0.109 | 30 |
| 31 | 0.478 | 21 | 0.100 | 25 | 3.090 | 10 | 0.057 | 23 |
| 17 | 3.210 | 12 | 1.273 | 47 | ND | 11 | 14.499 | 117 |
| 32 | 1.010 | 18 | 0.111 | 32 | ND | 9 | 0.322 | 31 |
| 47 | 1.411 | 60 | 0.082 | 50 | 0.560 | 71 | 0.208 | 70 |

### Experimental Example 2

### Blood Glucose-Lowering Effects of PPAR Agonist Compounds on Mice with Hyperglycemic Diabetes

1. Materials and Methods

Effects of PPAR compounds on a blood glucose level were evaluated in 7-week-old diabetic male mice (db/db mice). Blood was collected from caudal veins of the diabetic animals to which the drug was administered once a day for 5 consecutive days, and the blood glucose level was then measured with a blood glucose test meter (ACCU CHEK Active®).

2. Experimental Results

Experimental animals were orally administered with 5 PPAR compounds that exhibit partial agonism on PPAR-α and -γ in the in vitro reporter assay, and the PPAR-γ modulator INT-131, respectively. INT-131 as a control drug exhibited ED₃₀ of 4 mg/kg. The compound of formula 1 in accordance with the present invention was shown to have excellent hypoglycemic activity comparable to or higher than INT-131.

TABLE 2
Hypoglycemic activity of representative compounds as measured in vivo

| | | Blood glucose (mg/dL) | | |
|---|---|---|---|---|
| Ex. No. or | | | | % Inhibition vs. |
| Compounds | Dose (mg/kg) | D0 | D5 | Control |
| Control | — | 478 ± 23 | 486 ± 22 | — |
| INT-131 | 1 | 381 ± 30 | 350 ± 31 | 8 |
| | 3 | 382 ± 31 | 286 ± 34 | 25 |
| | 10 | 382 ± 28 | 237 ± 19 | 38 |
| Ex. 41 | 0.1 | 422 ± 34 | 455 ± 67 | 4 |
| | 0.3 | 422 ± 36 | 378 ± 44 | 20 |
| | 1 | 422 ± 30 | 304 ± 13 | 36 |
| | 3 | 423 ± 35 | 257 ± 40 | 46 |
| Ex. 48 | 1 | 452 ± 22 | 375 ± 25 | 24 |
| | 3 | 453 ± 20 | 347 ± 70 | 30 |
| | 10 | 453 ± 43 | 280 ± 40 | 43 |
| Ex. 21 | 0.1 | 485 ± 44 | 416 ± 45 | 16 |
| | 0.3 | 483 ± 38 | 354 ± 67 | 28 |
| | 1 | 482 ± 45 | 354 ± 58 | 29 |
| | 3 | 483 ± 45 | 319 ± 63 | 36 |
| Ex. 36 | 1 | 577 ± 15 | 381 ± 73 | 33 |
| | 10 | 577 ± 16 | 278 ± 56 | 62 |
| Ex. 32 | 0.3 | 500 ± 32 | 416 ± 53 | 21 |
| | 3 | 498 ± 30 | 205 ± 42 | 61 |

### Experimental Example 3

### Binding Capacity of Inventive Compounds with Trap220 Serving as a Main Cofactor in Adipogenic Differentiation

1. Materials and Methods

Following induction of transient intracellular expression of PPAR-γ and Trap220, the binding capacity of the inventive compounds to a cofactor Trap220 after activation of PPAR-γ by the action of the inventive compounds was evaluated (mammalian two-hybrid assay).

For this purpose, a monkey ovary cell line CHO-K1 (CCL-61, ATCC) was used as a test cell line. DNA constructs used in this assay were an expression vector pVP16 (Clontech) constructed to express a fusion of a human PPAR-γ2 ligand-binding domain with an activation domain of the yeast transactivator GAL4, and an expression vector pM (Clontech) constructed to express a fusion of human Trap220 with the GAL4 DNA-binding domain. Rosiglitazone maleate (Alcon Biosciences Private Limited), which is clinically used as a PPAR-γ agonist, was employed as a control drug.

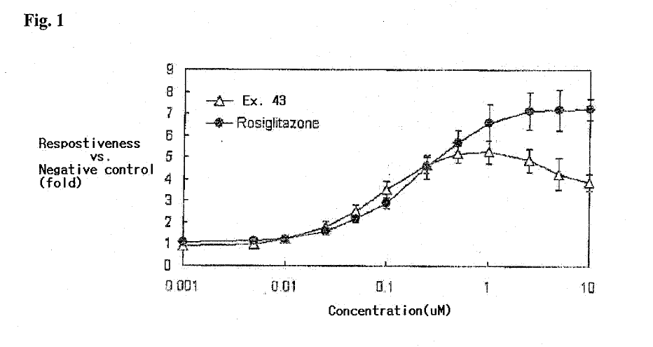

The CHO-K1 cells were transiently transfected with two DNA constructs expressing the chimeric receptors and DNA constructs comprising 5 copies of the GAL4 DNA-binding domain and capable of inducing expression of firefly luciferase or Renilla luciferase using a Lipofectamine Plus reagent (Invitrogen, USA). Subsequent processes were carried out in the same manner as in the transactivation assay. The experimental results were expressed as an increase of the responsiveness vs. the negative control group with no addition of the drug. The results thus obtained are shown in FIG. 1.

2. Experimental Results

10 compounds including Example 43 exhibited reduced responsiveness as compared to the positive control drug rosiglitazone, thus representing the results similar to those published for INT-131 undergoing II/III phase clinical trials according to the same mechanism. Therefore, these experimental results illustrate the mechanism background capable of alleviating adverse side effects associated with body weight gain, among adverse side effects of conventional drugs.

### INDUSTRIAL APPLICABILITY

As apparent from the above description, the compound of the present invention has modulatory activity on peroxisome proliferator-activated gamma receptor (PPAR-γ). That is, the compound shows hypoglycemic, hypolipidemic and insulin resistance-reducing effects on PPAR-mediated diseases or disorders, so it can be prophylactically or therapeutically effective for PPAR-related diseases and conditions, such as obesity, diabetes, hypertension, and hyperlipidemia.

1. A compound represented by formula 1, or a racemate, optical isomer or pharmaceutically acceptable salt thereof: 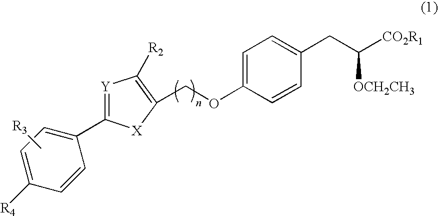
wherein:
R₁ is hydrogen, ethyl, or an alkali metal;
R₂ is hydrogen or methyl;
X is S or O;
Y is N or CH;
R₃ is hydrogen, lower alkyl or lower alkoxy;
R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and
n is an integer of 1 to 5.

2. The compound of claim 1, wherein 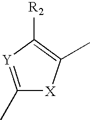
in formula 1 is selected from: 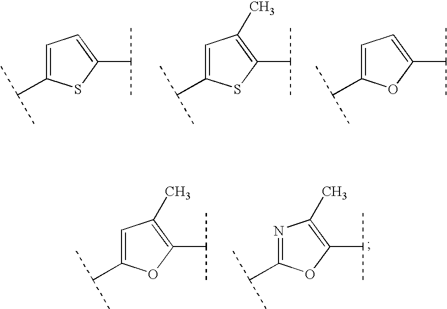
lower alkyl is selected from methyl, ethyl and isopropyl;
lower alkoxy is selected from methoxy and ethoxy;
halide is selected from Cl, F and Br;
alkylcarbamoyl is selected from: 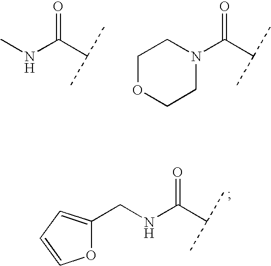
oxadiazole is selected from: 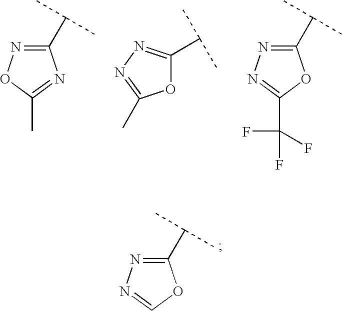
isoxazole is selected from: 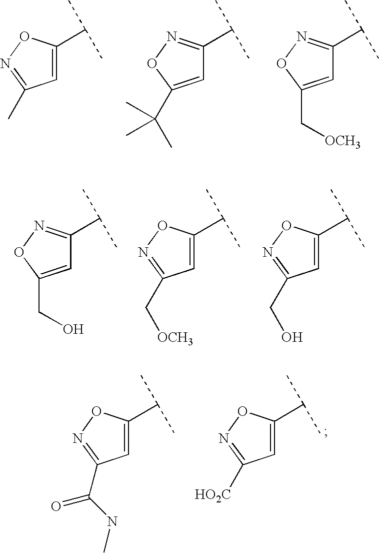
and
tetrazole is selected from: 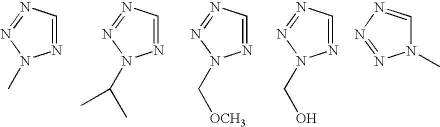
or a racemate, optical isomer or pharmaceutically acceptable salt thereof:

3. The compound of claim 1, wherein the compound of formula 1 is selected from the group consisting of:
(S)-2-ethoxy-3-(4-((5-(3-methoxyphenyl)-3-methylthiophen-2-yl)methoxy)phenyl)propionic acid,
(S)-2-ethoxy-3-(4-((5-(4-fluorophenyl)-3-methylthiophen-2-yl)methoxy)phenyl)propionic acid,
(S)-3-(4-((5-(3,4-dimethoxyphenyl)-3-methylthiophen-2-yl)methoxy)phenyl)-2-ethoxypropionic acid,
(S)-2-ethoxy-3-(4-((5-(4-methoxyphenyl)-3-methylthiophen-2-yl)methoxy)phenyl)propionic acid,
(S)-2-ethoxy-3-(4-((5-(4-ethylphenyl)-3-methylthiophen-2-yl)methoxy)phenyl)propionic acid,
(S)-2-ethoxy-3-(4-((3-methyl-5-(4-(trifluoromethyl)phenyl)thiophen-2-yl)methoxy)phenyl)propionic acid,
(S)-2-ethoxy-3-(4-((3-methyl-5-p-phenylthiophen-2-yl)methoxy)phenyl)propionic acid,
(S)-2-ethoxy-3-(4-((3-methyl-5-(4-(trifluoromethoxy)phenyl)thiophen-2-yl)methoxy)phenyl)propionic acid,
(S)-2-ethoxy-3-(4-((5-(4-isopropylphenyl)-3-methylthiophen-2-yl)methoxy)phenyl)propionic acid,
(S)-2-ethoxy-3-(4-((3-methyl-5-phenylthiophen-2-yl)methoxy)phenyl)propionic acid,
(S)-3-(4-((5-(4-cyanophenyl)-3-methylthiophen-2-yl)methoxy)phenyl)-2-ethoxypropionic acid,
(S)-3-(4-((5-(4-acetylphenyl)-3-phenylthiophen-2-yl)methoxy)phenyl)-2-ethoxypropionic acid,
(S)-3-(4-((5-(4-acetamidophenyl)-3-methylthiophen-2-yl)methoxy)phenyl)-2-ethoxypropionic acid,
(S)-2-ethoxy-3-(4-((3-methyl-5-(4-(N-methylacetamido)phenyl)thiophen-2-yl)methoxy)phenyl)propionic acid,
(S)-3-(4-((5-(4-benzoylphenyl)-3-methylthiophen-2-yl)methoxy)phenyl)-2-ethoxypropionic acid,
(S)-2-ethoxy-3-(4-((5-(4-(furan-2-yl-methylcarbamoyl)phenyl)-3-methylthiophen-2-yl)methoxy)phenyl)propionic acid,
(S)-2-ethoxy-3-(4-((3-methyl-5-(4-(morpholine-4-carbonyl)phenyl)thiophen-2-yl)methoxy)phenyl)propionic acid,
(S)-2-ethoxy-3-(4-((3-methyl-5-(4-(morpholinosulfonyl)phenyl)thiophen-2-yl)methoxy)phenyl)propionic acid,
(S)-3-(4-((5-(4-(5,6-dihydro-4H-1,3-oxazin-2-yl)phenyl)-3-methylthiophen-2-yl)methoxy)phenyl)-2-ethoxypropionic acid,
(S)-2-ethoxy-3-(4-((3-methyl-5-(4-morpholinophenyl)thiophen-2-yl)methoxy)phenyl)propionic acid,
(S)-2-ethoxy-3-(4-((3-methyl-5-(4-(2-methylthiazol-4-yl)phenyl)thiophen-2-yl)methoxy)phenyl)propionic acid,
(S)-2-ethoxy-3-(4-((3-methyl-5-(4-(1-methyl-6-oxo-1,4,5,6-tetrahydropyridazin-3-yl)phenyl)thiophen-2-yl)methoxy)phenyl)propionic acid,
(S)-3-(4-((5-(4-(2H-benzo[b][1,4]oxazin-3-yl)phenyl)-3-methylthiophen-2-yl)methoxy)phenyl)-2-ethoxypropionic acid,
(S)-3-(4-((5-(4-(1,2,3-thiadiazol-4-yl)phenyl)-3-methylthiophen-2-yl)methoxy)phenyl)-2-ethoxypropionic acid,
(S)-2-ethoxy-3-(4-((3-methyl-5-(4-(5-methyl-1,2,4-oxadiazol-3-yl)phenyl)thiophen-2-yl)methoxy)phenyl)propionic acid,
(S)-2-ethoxy-3-(4-((3-methyl-5-(4-(5-methyl-1,3,4-oxadiazol-2-yl)phenyl)thiophen-2-yl)methoxy)phenyl)propionic acid,
(S)-2-ethoxy-3-(4-((3-methyl-5-(4-(5-(trifluoromethyl)-1,3,4-oxadiazol-2-yl)phenyl)thiophen-2-yl)methoxy)phenyl)propionic acid,
(S)-3-(4-((5-(4-(1,3,4-oxadiazol-2-yl)phenyl)-3-methylthiophen-2-yl)methoxy)phenyl)-2-ethoxypropionic acid,
(S)-2-ethoxy-3-(4-((3-methyl-5-(4-(2-methyl-2H-tetrazol-5-yl)phenyl)thiophen-2-yl)methoxy)phenyl)propionic acid,
(S)-2-ethoxy-3-(4-((3-methyl-5-(4-(1-methyl-1H-tetrazol-5-yl)phenyl)thiophen-2-yl)methoxy)phenyl)propionic acid,
(S)-2-ethoxy-3-(4-((5-(4-(2-isopropyl-2H-tetrazol-5-yl)phenyl)-3-methylthiophen-2-yl)methoxy)phenyl)propionic acid,
(S)-2-ethoxy-3-(4-((5-(4-(2-(methoxymethyl)-2H-tetrazol-5-yl)phenyl)-3-methylthiophen-2-yl)methoxy)phenyl)propionic acid,
(S)-2-ethoxy-3-(4-((5-(4-(2-(hydroxymethyl)-2H-tetrazol-5-yl)phenyl)-3-methylthiophen-2-yl)methoxy)phenyl)propionic acid,
(S)-3-(4-((5-(4-(4,5-dimethyloxazol-2-yl)phenyl)-3-methylthiophen-2-yl)methoxy)phenyl)-2-ethoxypropionic acid,
(S)-2-ethoxy-3-(4-((5-(4-(5-(hydroxymethyl)isoxazol-3-yl)phenyl)-3-methylthiophen-2-yl)methoxy)phenyl)propionic acid,
(S)-2-ethoxy-3-(4-((5-(4-(5-(methoxymethyl)isoxazol-3-yl)phenyl)-3-methylthiophen-2-yl)methoxy)phenyl)propionic acid,
(S)-5-(4-(5-((4-(2-carbonyl-2-ethoxyethyl)phenoxy)methyl)-4-methylthiophen-2-yl)phenyl)isoxazole-3-carboxylic acid,
(S)-2-ethoxy-3-(4-((3-methyl-5-(4-(3-(methylcarbamoyl)isoxazol-5-yl)phenyl)thiophen-2-yl)methoxy)phenyl)propionic acid,
(S)-2-ethoxy-3-(4-((5-(4-(3-(hydroxymethyl)isoxazol-5-yl)phenyl)-3-methylthiophen-2-yl)methoxy)phenyl)propionic acid,
(S)-2-ethoxy-3-(4-((5-(4-(3-(methoxymethyl)isoxazol-5-yl)phenyl)-3-methylthiophen-2-yl)methoxy)phenyl)propionic acid,
(S)-2-ethoxy-3-(4-((3-methyl-5-(4-(3-methylisoxazol-5-yl)phenyl)thiophen-2-yl)methoxy)phenyl)propionic acid,
lithium (S)-2-ethoxy-3-(4-((3-methyl-5-(4-(3-methylisoxazol-5-yl)phenyl)thiophen-2-yl)methoxy)phenyl)propanoate,
sodium (S)-2-ethoxy-3-(4-((3-methyl-5-(4-(3-methylisoxazol-5-yl)phenyl)thiophen-2-yl)methoxy)phenyl)propanoate,
potassium (S)-2-ethoxy-3-(4-((3-methyl-5-(4-(3-methylisoxazol-5-yl)phenyl)thiophen-2-yl)methoxy)phenyl)propanoate,
(S)-2-ethoxy-3-(4-((5-(4-(3-methylisoxazol-5-yl)phenyl)furan-2-yl)methoxy)phenyl)propionic acid,
(S)-2-ethoxy-3-(4-((3-methyl-5-(4-(3-methylisoxazol-5-yl)phenyl)furan-2-yl)methoxy)phenyl)propionic acid,
(S)-2-ethoxy-3-(4-((4-methyl-2-(4-(3-methylisoxazol-5-yl)phenyl)thiazol-5-yl)methoxy)phenyl)propionic acid,
(S)-3-(4-((5-(4-(5-tert-butylisoxazol-3-yl)phenyl)-3-methylthiophen-2-yl)methoxy)phenyl)-2-ethoxypropionic acid,
lithium (S)-3-(4-((5-(4-(5-tert-butylisoxazol-3-yl)phenyl)-3-methylthiophen-2-yl)methoxy)phenyl)-2-ethoxypropanoate,
sodium (S)-3-(4-((5-(4-(5-tert-butylisoxazol-3-yl)phenyl)-3-methylthiophen-2-yl)methoxy)phenyl)-2-ethoxypropanoate,
potassium (S)-3-(4-((5-(4-(5-tert-butylisoxazol-3-yl)phenyl)-3-methylthiophen-2-yl)methoxy)phenyl)-2-ethoxypropanoate,
(S)-3-(4-((5-(4-(5-tert-butylisoxazol-3-yl)phenyl)furan-2-yl)methoxy)phenyl)-2-ethoxypropionic acid,
(S)-3-(4-((5-(4-(5-tert-butylisoxazol-3-yl)phenyl)-3-methylfuran-2-yl)methoxy)phenyl)-2-ethoxypropionic acid, and
(S)-3-(4-((2-(4-(5-tert-butylisoxazol-3-yl)phenyl)-4-methylthiazol-5-yl)methoxy)phenyl)-2-ethoxypropionic acid.

4. A method for preparing a compound of formula 1, comprising:
(1) reacting a compound of formula 2 with a compound of formula 3, 4, 5 or 6 to form a compound of formula 7, 8, 9 or 10; and
(2) reacting the compound of formula 7, 8, 9 or 10 with a boron compound of formula 11 to form a compound of formula 1 wherein R₁ is ethyl: 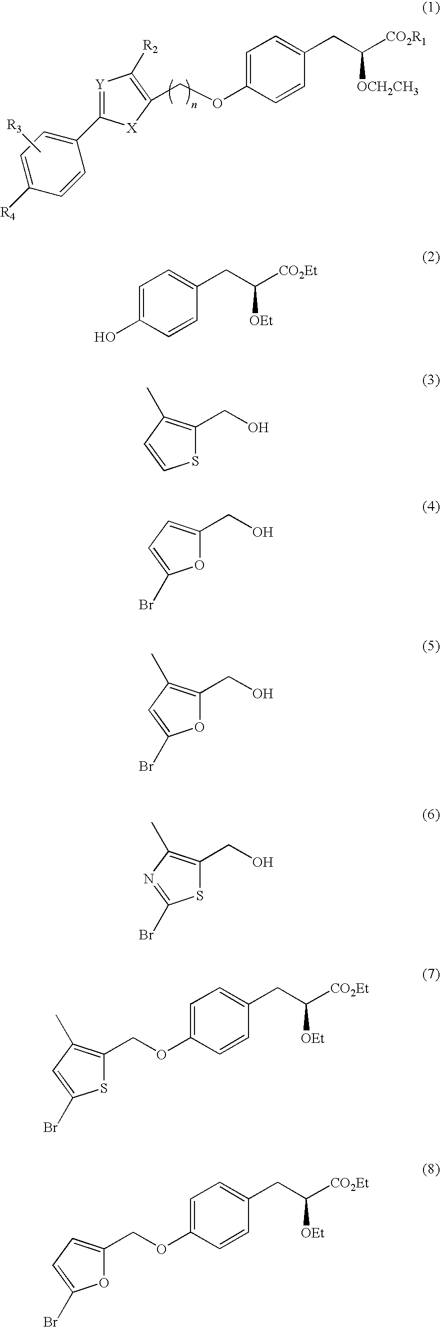 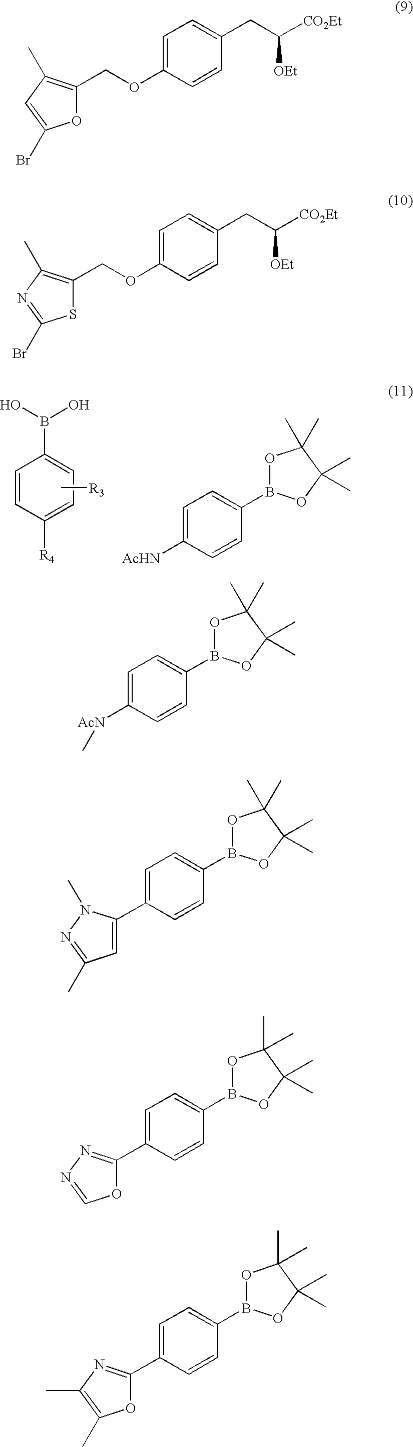 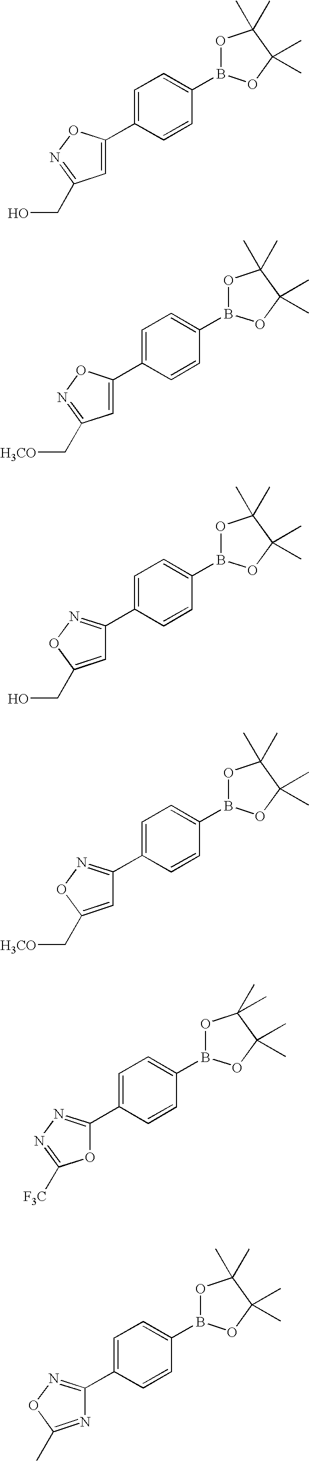 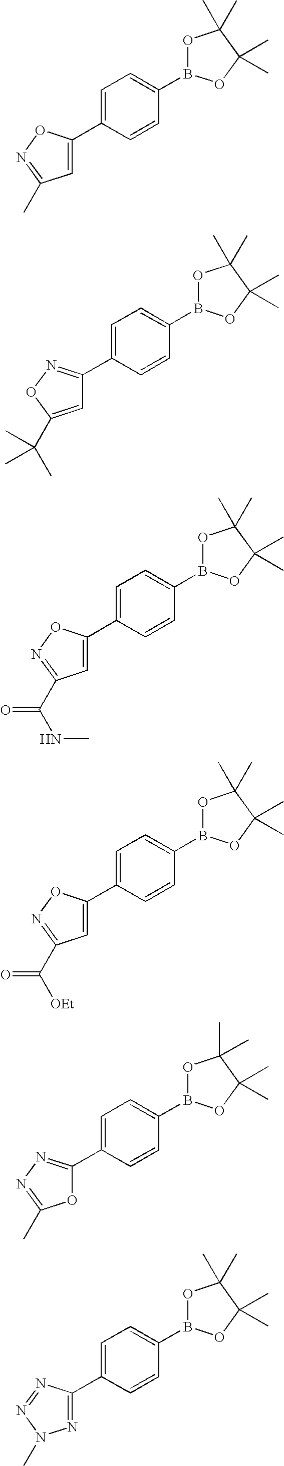 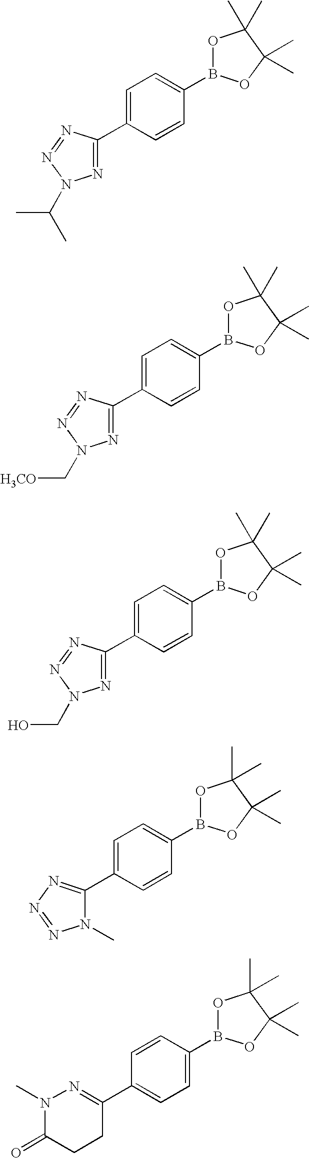
wherein:
R₁ is hydrogen, ethyl, or an alkali metal;
R₂ is hydrogen or methyl;
X is S or O;
Y is N or CH;
R₃ is hydrogen, lower alkyl or lower alkoxy;
R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and
n is an integer of 1 to 5.

5. The method of claim 4, wherein
Step 1 includes nucleophilic substitution of Compound 2 with Compound 3 through the Mitsunobu reaction to form an ether bond, followed by bromination of the reaction product with N-bromosuccinimide to form Compound 7,
Step 2 includes Suzuki coupling of Compound 7 using boronic acid or dioxaborolan of formula 11 and a palladium catalyst to form a carbon-carbon bond, and
the resulting compound of formula 1 is a compound of formula 1-1: 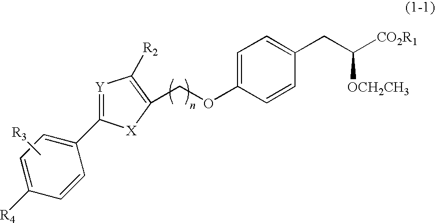
wherein R₁ is ethyl; X is S; Y is CH; R₂ is methyl; R₃ and R₄ are as defined in claim 4; and n is 1.

6. The method of claim 4, wherein
Step 1 includes nucleophilic substitution of Compound 2 with Compound 4 through the Mitsunobu reaction to form Compound 8 via formation of an ether bond,
Step 2 includes Suzuki coupling of Compound 8 using boronic acid or dioxaborolan of formula 11 and a palladium catalyst to form a carbon-carbon bond, and
the resulting compound of formula 1 is a compound of formula 1-2: 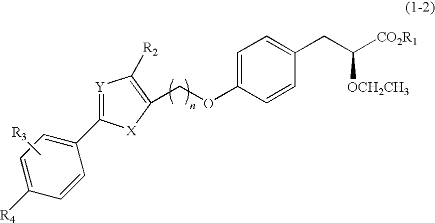
wherein R₁ is ethyl; X is O; Y is CH; R₂ is hydrogen; R₃ and R₄ are as defined in claim 4, and n is 1.

7. The method of claim 4, wherein
Step 1 includes nucleophilic substitution of Compound 2 with Compound 5 through the Mitsunobu reaction to form Compound 9 via formation of an ether bond,
Step 2 includes Suzuki coupling of Compound 9 using boronic acid or dioxaborolan of formula 11 and a palladium catalyst to form a carbon-carbon bond, and
the resulting compound of formula 1 is a compound of formula 1-3: 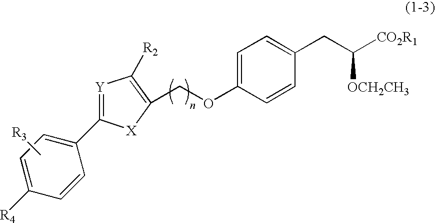
wherein R₁ is ethyl; X is O; Y is CH; R₂ is methyl; R₃ and R₄ are as defined in claim 4; and n is 1.

8. The method of claim 4, wherein
Step 1 includes nucleophilic substitution of Compound 2 with Compound 6 through the Mitsunobu reaction to form Compound 10 via formation of an ether bond,
Step 2 includes Suzuki coupling of Compound 10 using boronic acid or dioxaborolan of formula 11 and a palladium catalyst to form a carbon-carbon bond, and
the resulting compound of formula 1 is a compound of formula 1-4: 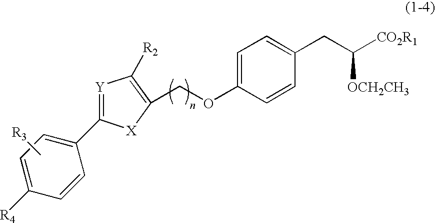
wherein R₁ is ethyl; X is S; Y is N; R₂ is methyl; R₃ and R₄ are as defined in claim 4; and n is 1.

9. The method of claim 4, further comprising hydrolysis of the reaction product of Step 2 after reaction with a boron compound to form a compound of formula 1 wherein R₁ is hydrogen.

10. The method of claim 9, wherein
Step 1 includes nucleophilic substitution of Compound 2 with Compound 3 through the Mitsunobu reaction to form an ether bond, followed by bromination of the reaction product with N-bromosuccinimide to form Compound 7,
Step 2 includes Suzuki coupling of Compound 7 using boronic acid or dioxaborolan of formula 11 and a palladium catalyst to form a carbon-carbon bond, followed by hydrolysis, and
the resulting compound of formula 1 is a compound of formula 1-5: 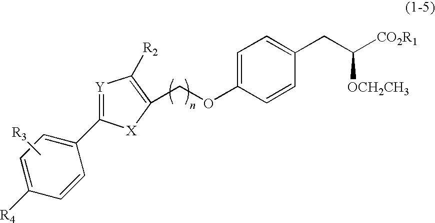
wherein R₁ is hydrogen; X is S; Y is CH; R₂ is methyl; R₃ and R₄ are as defined in claim 4; and n is 1.

11. The method of claim 9, wherein
Step 1 includes nucleophilic substitution of Compound 2 with Compound 4 through the Mitsunobu reaction to form Compound 8 via formation of an ether bond,
Step 2 includes Suzuki coupling of Compound 8 using boronic acid or dioxaborolan of formula 11 and a palladium catalyst to form a carbon-carbon bond, followed by hydrolysis, and
the resulting compound of formula 1 is a compound of formula 1-6: 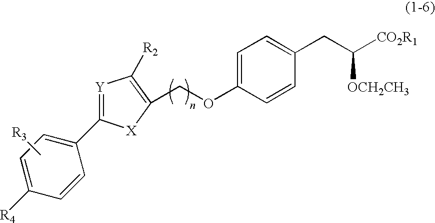
wherein R₁ is hydrogen; X is O; Y is CH; R₂ is hydrogen; R₃ and R₄ are as defined in claim 4; and n is 1.

12. The method of claim 9, wherein
Step 1 includes nucleophilic substitution of Compound 2 with Compound 5 through the Mitsunobu reaction to form Compound 9 via formation of an ether bond,
Step 2 includes Suzuki coupling of Compound 9 using boronic acid or dioxaborolan of formula 11 and a palladium catalyst to form a carbon-carbon bond, followed by hydrolysis, and
the resulting compound of formula 1 is a compound of formula 1-7: 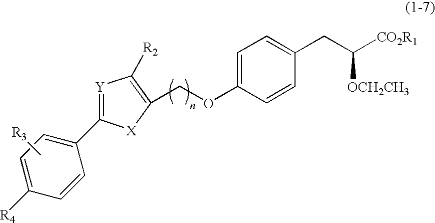
wherein R₁ is hydrogen; X is O; Y is CH; R₂ is methyl; R₃ and R₄ are as defined in claim 4; and n is 1.

13. The method of claim 9, wherein
Step 1 includes nucleophilic substitution of Compound 2 with Compound 6 through the Mitsunobu reaction to form Compound 10 via formation of an ether bond,
Step 2 includes Suzuki coupling of Compound 10 using boronic acid or dioxaborolan of formula 11 and a palladium catalyst to form a carbon-carbon bond, followed by hydrolysis, and
the resulting compound of formula 1 is a compound of formula 1-8: 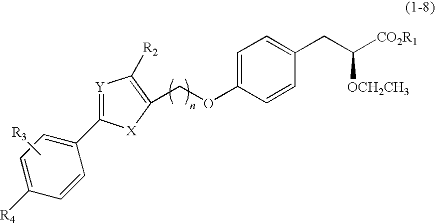
wherein R₁ is hydrogen; X is S; Y is N; R₂ is methyl; R₃ and R₄ are as defined in claim 4; and n is 1.

14. The method of claim 9, wherein the hydrolysate is reacted with sodium, lithium or potassium ethyl-2 hexanoate to prepare a compound of formula 1 wherein R₁ is an alkali metal.

15. The method of claim 14, wherein
Step 1 includes nucleophilic substitution of Compound 2 with Compound 3 through the Mitsunobu reaction to form an ether bond, followed by bromination of the reaction product with N-bromosuccinimide to form Compound 7,
Step 2 includes Suzuki coupling of Compound 7 using boronic acid or dioxaborolan of formula 11 and a palladium catalyst to form a carbon-carbon bond, followed by hydrolysis, and
the resulting compound of formula 1 is a compound of formula 1-9: 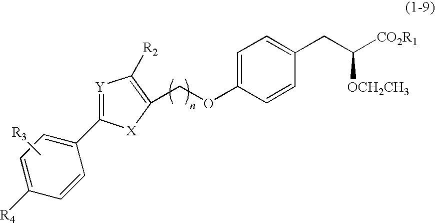
wherein R₁ is an alkali metal; X is S; Y is CH; R₂ is methyl; R₃ and R₄ are as defined in claim 4; and n is 1.

16. The method of claim 14, wherein
Step 1 includes nucleophilic substitution of Compound 2 with Compound 4 through the Mitsunobu reaction to form Compound 8 via formation of an ether bond,
Step 2 includes Suzuki coupling of Compound 8 using boronic acid or dioxaborolan of formula 11 and a palladium catalyst to form a carbon-carbon bond, followed by hydrolysis, and
the resulting compound of formula 1 is a compound of formula 1-10: 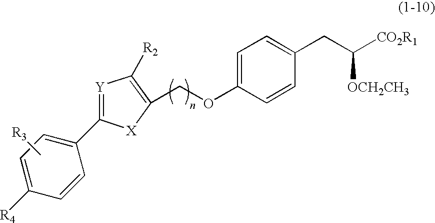
wherein R₁ is an alkali metal; X is O; Y is CH; R₂ is hydrogen; R₃ and R₄ are as defined in claim 4; and n is 1.

17. The method of claim 14, wherein
Step 1 includes nucleophilic substitution of Compound 2 with Compound 5 through the Mitsunobu reaction to form Compound 9 via formation of an ether bond,
Step 2 includes Suzuki coupling of Compound 9 using boronic acid or dioxaborolan of formula 11 and a palladium catalyst to form a carbon-carbon bond, followed by hydrolysis, and
the resulting compound of formula 1 is a compound of formula 1-11: 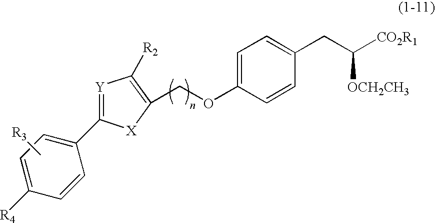
wherein R₁ is an alkali metal; X is O; Y is CH; R₂ is methyl; R₃ and R₄ are as defined in claim 4; and n is 1.

18. The method of claim 14, wherein
Step 1 includes nucleophilic substitution of Compound 2 with Compound 6 through the Mitsunobu reaction to form Compound 10 via formation of an ether bond,
Step 2 includes Suzuki coupling of Compound 10 using boronic acid or dioxaborolan of formula 11 and a palladium catalyst to form a carbon-carbon bond, followed by hydrolysis, and
the resulting compound of formula 1 is a compound of formula 1-12: 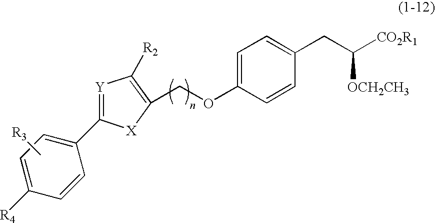
wherein R₁ is an alkali metal; X is S; Y is N; R₂ is methyl; R₃ and R₄ are as defined in claim 4; and n is 1.

19. A pharmaceutical composition for modulation of peroxisome proliferator-activated receptor gamma (PPAR-γ), comprising a compound represented by formula 1, an optical isomer thereof or a pharmaceutically acceptable salt thereof, as an active ingredient: 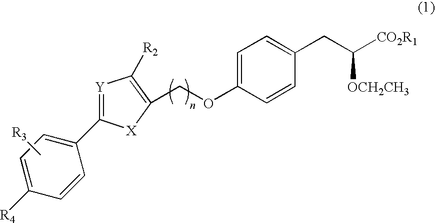
wherein:
R₁ is hydrogen, ethyl, or an alkali metal;
R₂ is hydrogen or methyl;
X is S or O;
Y is N or CH;
R₃ is hydrogen, lower alkyl or lower alkoxy;
R₄ is hydrogen, lower alkyl, lower alkoxy, halide, cyano, acetyl, acetamino, benzoyl, carbamoyl, alkylcarbamoyl, aminosulfonyl, 2-H-benzo[b][1,4]oxazine, morpholine, thiazole, morpholinosulfonyl, morpholinocarbonyl, 4,5-dihydropyridazin-3(2H)-one, thiadiazole, oxadiazole, tetrazole, oxazole, or isoxazole, each of which being optionally substituted by at least one selected from the group consisting of hydrogen, halogen, C₁₋₆ alkyl, C₁₋₆ alkoxy, hydroxy, amino, trifluoromethyl, phenyl, benzyl, benzoyl, furan, thiophene, piperidine and morpholine; and
n is an integer of 1 to 5.

20. The composition of claim 19, wherein the compound represented by formula 1 is used for prevention or treatment of diabetes mellitus or metabolic syndrome, or a racemate, optical isomer or pharmaceutically acceptable salt thereof.

21. The composition of claim 19, wherein the compound represented by formula 1 is selected from the group consisting of:
(S)-2-ethoxy-3-(4-((5-(3-methoxyphenyl)-3-methylthiophen-2-yl)methoxy)phenyl)propionic acid,
(S)-2-ethoxy-3-(4-((5-(4-fluorophenyl)-3-methylthiophen-2-yl)methoxy)phenyl)propionic acid,
(S)-3-(4-((5-(3,4-dimethoxyphenyl)-3-methylthiophen-2-yl)methoxy)phenyl)-2-ethoxypropionic acid,
(S)-2-ethoxy-3-(4-((5-(4-methoxyphenyl)-3-methylthiophen-2-yl)methoxy)phenyl)propionic acid,
(S)-2-ethoxy-3-(4-((5-(4-ethylphenyl)-3-methylthiophen-2-yl)methoxy)phenyl)propionic acid,
(S)-2-ethoxy-3-(4-((3-methyl-5-(4-(trifluoromethyl)phenyl)thiophen-2-yl)methoxy)phenyl)propionic acid,
(S)-2-ethoxy-3-(4-((3-methyl-5-p-phenylthiophen-2-yl)methoxy)phenyl)propionic acid,
(S)-2-ethoxy-3-(4-((3-methyl-5-(4-(trifluoromethoxy)phenyl)thiophen-2-yl)methoxy)phenyl)propionic acid,
(S)-2-ethoxy-3-(4-((5-(4-isopropylphenyl)-3-methylthiophen-2-yl)methoxy)phenyl)propionic acid,
(S)-2-ethoxy-3-(4-((3-methyl-5-phenylthiophen-2-yl)methoxy)phenyl)propionic acid,
(S)-3-(4-((5-(4-cyanophenyl)-3-methylthiophen-2-yl)methoxy)phenyl)-2-ethoxypropionic acid,
(S)-3-(4-((5-(4-acetylphenyl)-3-phenylthiophen-2-yl)methoxy)phenyl)-2-ethoxypropionic acid,
(S)-3-(4-((5-(4-acetamidophenyl)-3-methylthiophen-2-yl)methoxy)phenyl)-2-ethoxypropionic acid,
(S)-2-ethoxy-3-(4-((3-methyl-5-(4-(N-methylacetamido)phenyl)thiophen-2-yl)methoxy)phenyl)propionic acid,
(S)-3-(4-((5-(4-benzoylphenyl)-3-methylthiophen-2-yl)methoxy)phenyl)-2-ethoxypropionic acid,
(S)-2-ethoxy-3-(4-((5-(4-(furan-2-yl-methylcarbamoyl)phenyl)-3-methylthiophen-2-yl)methoxy)phenyl)propionic acid,
(S)-2-ethoxy-3-(4-((3-methyl-5-(4-(morpholine-4-carbonyl)phenyl)thiophen-2-yl)methoxy)phenyl)propionic acid,
(S)-2-ethoxy-3-(4-((3-methyl-5-(4-(morpholinosulfonyl)phenyl)thiophen-2-yl)methoxy)phenyl)propionic acid,
(S)-3-(4-((5-(4-(5,6-dihydro-4H-1,3-oxazin-2-yl)phenyl)-3-methylthiophen-2-yl)methoxy)phenyl)-2-ethoxypropionic acid,
(S)-2-ethoxy-3-(4-((3-methyl-5-(4-morpholinophenyl)thiophen-2-yl)methoxy)phenyl)propionic acid,
(S)-2-ethoxy-3-(4-((3-methyl-5-(4-(2-methylthiazol-4-yl)phenyl)thiophen-2-yl)methoxy)phenyl)propionic acid,
(S)-2-ethoxy-3-(4-((3-methyl-5-(4-(1-methyl-6-oxo-1,4,5,6-tetrahydropyridazin-3-yl)phenyl)thiophen-2-yl)methoxy)phenyl)propionic acid,
(S)-3-(4-((5-(4-(2H-benzo[b][1,4]oxazin-3-yl)phenyl)-3-methylthiophen-2-yl)methoxy)phenyl)-2-ethoxypropionic acid,
(S)-3-(4-((5-(4-(1,2,3-thiadiazol-4-yl)phenyl)-3-methylthiophen-2-yl)methoxy)phenyl)-2-ethoxypropionic acid,
(S)-2-ethoxy-3-(4-((3-methyl-5-(4-(5-methyl-1,2,4-oxadiazol-3-yl)phenyl)thiophen-2-yl)methoxy)phenyl)propionic acid,
(S)-2-ethoxy-3-(4-((3-methyl-5-(4-(5-methyl-1,3,4-oxadiazol-2-yl)phenyl)thiophen-2-yl)methoxy)phenyl)propionic acid,
(S)-2-ethoxy-3-(4-((3-methyl-5-(4-(5-(trifluoromethyl)-1,3,4-oxadiazol-2-yl)phenyl)thiophen-2-yl)methoxy)phenyl)propionic acid,
(S)-3-(4-((5-(4-(1,3,4-oxadiazol-2-yl)phenyl)-3-methylthiophen-2-yl)methoxy)phenyl)-2-ethoxypropionic acid,
(S)-2-ethoxy-3-(4-((3-methyl-5-(4-(2-methyl-2H-tetrazol-5-yl)phenyl)thiophen-2-yl)methoxy)phenyl)propionic acid,
(S)-2-ethoxy-3-(4-((3-methyl-5-(4-(1-methyl-1H-tetrazol-5-yl)phenyl)thiophen-2-yl)methoxy)phenyl)propionic acid,
(S)-2-ethoxy-3-(4-((5-(4-(2-isopropyl-2H-tetrazol-5-yl)phenyl)-3-methylthiophen-2-yl)methoxy)phenyl)propionic acid,
(S)-2-ethoxy-3-(4-((5-(4-(2-(methoxymethyl)-2H-tetrazol-5-yl)phenyl)-3-methylthiophen-2-yl)methoxy)phenyl)propionic acid,
(S)-2-ethoxy-3-(4-((5-(4-(2-(hydroxymethyl)-2H-tetrazol-5-yl)phenyl)-3-methylthiophen-2-yl)methoxy)phenyl)propionic acid,
(S)-3-(4-((5-(4-(4,5-dimethyloxazol-2-yl)phenyl)-3-methylthiophen-2-yl)methoxy)phenyl)-2-ethoxypropionic acid,
(S)-2-ethoxy-3-(4-((5-(4-(5-(hydroxymethyl)isoxazol-3-yl)phenyl)-3-methylthiophen-2-yl)methoxy)phenyl)propionic acid,
(S)-2-ethoxy-3-(4-((5-(4-(5-(methoxymethyl)isoxazol-3-yl)phenyl)-3-methylthiophen-2-yl)methoxy)phenyl)propionic acid,
(S)-5-(4-(5-((4-(2-carbonyl-2-ethoxyethyl)phenoxy)methyl)-4-methylthiophen-2-yl)phenyl)isoxazole-3-carboxylic acid,
(S)-2-ethoxy-3-(4-((3-methyl-5-(4-(3-(methylcarbamoyl)isoxazol-5-yl)phenyl)thiophen-2-yl)methoxy)phenyl)propionic acid,
(S)-2-ethoxy-3-(4-((5-(4-(3-(hydroxymethyl)isoxazol-5-yl)phenyl)-3-methylthiophen-2-yl)methoxy)phenyl)propionic acid,
(S)-2-ethoxy-3-(4-((5-(4-(3-(methoxymethyl)isoxazol-5-yl)phenyl)-3-methylthiophen-2-yl)methoxy)phenyl)propionic acid,
(S)-2-ethoxy-3-(4-((3-methyl-5-(4-(3-methylisoxazol-5-yl)phenyl)thiophen-2-yl)methoxy)phenyl)propionic acid,
lithium (S)-2-ethoxy-3-(4-((3-methyl-5-(4-(3-methylisoxazol-5-yl)phenyl)thiophen-2-yl)methoxy)phenyl)propanoate,
sodium (S)-2-ethoxy-3-(4-((3-methyl-5-(4-(3-methylisoxazol-5-yl)phenyl)thiophen-2-yl)methoxy)phenyl)propanoate,
potassium (S)-2-ethoxy-3-(4-((3-methyl-5-(4-(3-methylisoxazol-5-yl)phenyl)thiophen-2-yl)methoxy)phenyl)propanoate,
(S)-2-ethoxy-3-(4-((5-(4-(3-methylisoxazol-5-yl)phenyl)furan-2-yl)methoxy)phenyl)propionic acid,
(S)-2-ethoxy-3-(4-((3-methyl-5-(4-(3-methylisoxazol-5-yl)phenyl)furan-2-yl)methoxy)phenyl)propionic acid,
(S)-2-ethoxy-3-(4-((4-methyl-2-(4-(3-methylisoxazol-5-yl)phenyl)thiazol-5-yl)methoxy)phenyl)propionic acid,
(S)-3-(4-((5-(4-(5-tert-butylisoxazol-3-yl)phenyl)-3-methylthiophen-2-yl)methoxy)phenyl)-2-ethoxypropionic acid,
lithium (S)-3-(4-((5-(4-(5-tert-butylisoxazol-3-yl)phenyl)-3-methylthiophen-2-yl)methoxy)phenyl)-2-ethoxypropanoate,
sodium (S)-3-(4-((5-(4-(5-tert-butylisoxazol-3-yl)phenyl)-3-methylthiophen-2-yl)methoxy)phenyl)-2-ethoxypropanoate,
potassium (S)-3-(4-((5-(4-(5-tert-butylisoxazol-3-yl)phenyl)-3-methylthiophen-2-yl)methoxy)phenyl)-2-ethoxypropanoate,
(S)-3-(4-((5-(4-(5-tert-butylisoxazol-3-yl)phenyl)furan-2-yl)methoxy)phenyl)-2-ethoxypropionic acid,
(S)-3-(4-((5-(4-(5-tert-butylisoxazol-3-yl)phenyl)-3-methylfuran-2-yl)methoxy)phenyl)-2-ethoxypropionic acid, and
(S)-3-(4-((2-(4-(5-tert-butylisoxazol-3-yl)phenyl)-4-methylthiazol-5-yl)methoxy)phenyl)-2-ethoxypropionic acid.

22. A use of a composition of claim 19 for modulation of peroxisome proliferator-activated receptor gamma (PPAR-γ).

23. A method for modulation of peroxisome proliferator-activated receptor gamma (PPAR-γ), comprising administering a composition of claim 19 to a subject.
